(19) United States
(12) Patent Application Publication
(10) Pub. No.: US 2025/0032580 A1
(43) Pub. Date: Jan. 30, 2025

(54) USE OF ADNF POLYPEPTIDES IN THERAPY

(71) Applicant: RAMOT AT TEL-AVIV UNIVERSITY LTD., Tel Aviv (IL)

(72) Inventor: Illana GOZES, Tel Aviv (IL)

(21) Appl. No.: 18/552,263

(22) PCT Filed: Mar. 25, 2022
(86) PCT No.: PCT/IL2022/050333

§ 371 Date: Sep. 25, 2023

Related U.S. Application Data
(60) Provisional application No. 63/165,819, filed on Mar. 25, 2021.

(51) Int. Cl. A61K 38/18; A61P 25/28; G01N 33/68

(52) U.S. Cl. CPC A61K 38/185; A61P 25/28; G01N 33/6896; G01N 2800/2814; G01N 2800/52

(57) ABSTRACT

Uses of ADNF polypeptides in therapy are provided. Accordingly, there is provided a method of treating a disease associated with visual evoked potential impairment and/or speech impairment that is not due to vocal disturbance and in which the subject suffers from the visual evoked potential impairment and/or speech impairment, the method comprising administering to the subject a therapeutically effective amount of an ADNF polypeptide, wherein said ADNF polypeptide has a neurotrophic/neuroprotective activity in an in vitro cortical neuron culture assay.

### RELATED APPLICATION/S

This application claims the benefit of priority of U.S. Provisional Patent Application No. 63/165,819 filed on 25 Mar. 2021, the contents of which are incorporated herein by reference in their entirety.

### SEQUENCE LISTING STATEMENT

The Sequence Listing in ASCII text file format, entitled 2024-03-25SequenceListing_GOZES8, created on Mar. 25, 2024, comprising 23,870 bytes, filed in the U.S. Patent and Trademark Office on even date herewith is incorporated herein by reference.

### FIELD AND BACKGROUND OF THE INVENTION

The present invention, in some embodiments thereof, relates to use of ADNF polypeptides in therapy.

Activity-dependent neuroprotective protein (also referred to as ADNP or ADNF III) is essential for brain formation and function [Bassan M, et al. J Neurochem. 1999 March; 72(3):1283-93; Zamostiano, R. et al., J Biol Chem. 2001 Jan. 5; 276(1):708-14; Pinhasov A, et al. Brain Res Dev Brain Res. 2003 Aug. 12; 144(1):83-90]. ADNP was shown to function in key cellular activities including embryogenesis, autophagy, dendritic spine plasticity, axonal transport, alternative RNA-splicing, wnt signaling, autism-linked protein translation and chromatin remodeling. De novo mutations in ADNP lead to the autistic ADNP syndrome [Van Dijck A, et al. Biological psychiatry 2019, 85(4): 287-297; Gozes I. et al. Transl Psychiatry. 2017, 21; 7(2):e1043. doi: 10.1038/tp.2017.27; Helsmoortel C, et al. Nature genetics 2014, 46(4): 380-384] and somatic ADNP mutations may drive Alzheimer's disease (AD) tauopathy (Ivashko-Pachima Y, et al. Molecular psychiatry 2021, 26 (5):1619-1633. Epub 2019 Oct. 30]. Furthermore, a decrease in blood ADNP expression was linked to increased inflammation [Braitch M, et al. Neuroimmunomodulation 2010, 17(2): 120-125] and reduced cognitive functions [Malishkevich A, et al. Journal of Alzheimer's disease: JAD 2016, 50(1): 249-260]. ADNP is found in the nucleus being part of the SWItch/Sucrose Non-Fermentable (SWI/SNF) complex, which constitutes a major part of the chromatin remodeling complexes [Mandel S, Gozes I. J Biol Chem. 2007 Nov. 23; 282(47):34448-56]. In mature neurons, ADNP is found in the cytoplasm [Mandel S, et al. J Mol Neurosci 2008, 35(2): 127-141] in association with microtubules through interaction with the microtubule end binding proteins, EB1 and EB3 [Oz S, et al. Molecular psychiatry 2014, 19(10): 1115-1124]. In turn, EB1/EB3 interaction with ADNP have been linked to dendritic spine formation [Oz S, et al. Molecular psychiatry 2014, 19(10): 1115-1124; Hacohen-Kleiman G, et al. The Journal of clinical investigation 2018, 128(11): 4956-4969, Karmon G, et al., Biological Psychiatry 2021], axonal transport [Amram N, et al. Molecular psychiatry 2016; 21(10): 1467-1476], enhancement of Tau-microtubule binding [Ivashko-Pachima Y, et al. Molecular psychiatry 2017, 22(9): 1335-1344; Grigg I, et al. Translational psychiatry 2020, 10(1): 228; Ivashko-Pachima Y, et al. Molecular psychiatry 2021, 26 (5):1619-1633. Epub 2019 Oct. 30] and protection against tau hyperphosphorylation/tauopathy [Grigg I, et al. Translational psychiatry 2020, 10(1): 228; Ivashko-Pachima Y, et al. Molecular psychiatry 2021, 26 (5):1619-1633. Epub 2019 Oct. 30; Vulih-Shultzman I, et al. The Journal of pharmacology and experimental therapeutics 2007, 323(2): 438-449].

ADNP polypeptides, including a proline-rich 8-amino acid polypeptide known as NAP [NAPVSIPQ (SEQ ID NO: 2), also known as Davunetide or CP201] and uses thereof in neuroprotection and treating multiple disorders are the subject of patents and patent applications including International Application Publication No. WO1/92333, WO98/35042, WOOO/27875, WO00/53217, WO01/12654, WO2004/080957, WO2006/099739, WO2007/096859, WO2008/084483, WO2011/021186, WO2009/026687, WO2011/083461, WO2011/099011, WO2013/171595, WO2017/130190, WO2004/060309, WO2003/022226 and WO2010/075635; and U.S. Pat. Nos. U.S. Pat. Nos. 5,767,240, 6,174,862 and 6,613,740; herein each incorporated by reference in their entirety.

### Clinical Studies

NAP (SEQ ID NO 2) has not been previously approved for the treatment of the ADNP syndrome; however, clinical trials for other indications have been conducted [progressive supranuclear palsy (PSP), mild cognitive impairment (MCI), and schizophrenia].
NAP (CP201, davunetide, AL-108) was tested in the following clinical trials:
1. ClinicalTrials.gov identifier: NCT00422981—MCI
2. ClinicalTrials.gov identifier: NCT00505765—Schizophrenia
3. ClinicalTrials.gov identifier: NCT01056965—Tauopathies
4. ClinicalTrials.gov identifier: NCT01110720—PSP
5. ClinicalTrials.gov identifier:NCT00404014—MCI-Following Coronary Artery Bypass Graft Surgery

In general, all studies have proven safety and tolerance of NAP (SEQ ID NO 2) in hundreds of adult compromised patients. Efficacy was seen in enhancement of cognitive function and functional activities of daily living.

6. In ClinicalTrials.gov identifier: NCT01403519-Innovative Biomarkers in Alzheimer's Disease and Frontotemporal Dementia: Preventative and Personalized, ADNP levels were shown to correlate with disease status (e.g. cognitive impairments, and schizophrenia) and tauopathy. For more information, please see Gozes I, Front Neurol. 2020 Nov. 24; 11:608444.

Src homology 3 (SH3) domain-ligand association governs protein-protein interactions in a wide variety of biological processes such as enzyme activation/inactivation by intramolecular interactions, alteration of cellular concentration/localization of signaling components, and mediation of multi-protein complex assembly. Src homology 3 (SH3) domain is responsible for controlling the protein-protein interactions of signaling pathways regulating the cytoskeleton (Schlessinger J. Curr Opin Genet Dev. 1994, 4(1),25).

### SUMMARY OF THE INVENTION

According to an aspect of some embodiments of the present invention there is provided a method of treating a disease associated with visual evoked potential impairment and/or speech impairment that is not due to vocal disturbance and in which the subject suffers from the visual evoked potential impairment and/or the speech impairment, the method comprising administering to the subject a therapeutically effective amount of an ADNF polypeptide, wherein the ADNF polypeptide has a neurotrophic/neuroprotective activity in an in vitro cortical neuron culture assay, thereby treating the disease in the subject, wherein the disease is not ADNP syndrome.

According to an aspect of some embodiments of the present invention there is provided a method of treating a disease selected from the group consisting of autism spectrum disorder and intellectual disability in which the subject suffers from visual evoked potential impairment and/or speech impairment that is not due to vocal disturbance, the method comprising administering to the subject a therapeutically effective amount of an ADNF polypeptide, wherein the ADNF polypeptide has a neurotrophic/neuroprotective activity in an in vitro cortical neuron culture assay, thereby treating the disease in the subject, wherein the disorder is not ADNP syndrome.

According to an aspect of some embodiments of the present invention there is provided a method of treating Alzheimer's disease in which the subject suffers from visual evoked potential impairment and/or speech impairment that is not due to vocal disturbance, the method comprising administering to the subject a therapeutically effective amount of an ADNF polypeptide, wherein the ADNF polypeptide has a neurotrophic/neuroprotective activity in an in vitro cortical neuron culture assay, thereby treating the disease in the subject.

According to an aspect of some embodiments of the present invention there is provided a method of monitoring efficacy of treatment with an ADNF polypeptide in a subject diagnosed with a disease associated with visual evoked potential impairment and/or speech impairment that is not due to vocal disturbance and in which the subject suffers from the visual evoked potential impairment and/or the speech impairment, wherein the subject has a visual evoked potential impairment and/or a speech impairment that is not due to vocal disturbance, the method comprising determining a visual evoked potential and/or speech ability of the subject following the treatment with the ADNF polypeptide, wherein an improvement in the visual evoked potential and/or the speech ability following the treatment with the ADNF polypeptide indicates the treatment is efficacious.

According to an aspect of some embodiments of the present invention there is provided a method of monitoring efficacy of treatment with an ADNF polypeptide in a subject diagnosed with a disease selected from the group consisting of autism spectrum disorder and intellectual disability in which the subject suffers from visual evoked potential impairment and/or speech impairment that is not due to vocal disturbance, the method comprising determining a visual evoked potential and/or speech ability of the subject following the treatment with the ADNF polypeptide, wherein an improvement in the visual evoked potential and/or the speech ability following the treatment with the ADNF polypeptide indicates the treatment is efficacious.

According to some embodiments of the invention, the disease is not ADNP syndrome.

According to an aspect of some embodiments of the present invention there is provided a method of monitoring efficacy of treatment with an ADNF polypeptide in a subject diagnosed with Alzheimer's disease in which the subject suffers from visual evoked potential impairment and/or speech impairment that is not due to vocal disturbance, the method comprising determining a visual evoked potential and/or speech ability of the subject following the treatment with the ADNF polypeptide, wherein an improvement in the visual evoked potential and/or the speech ability following the treatment with the ADNF polypeptide indicates the treatment is efficacious.

According to some embodiments of the invention, the speech impairment or ability is determined by syntax complexity.

According to some embodiments of the invention, the disease is selected from the group consisting of autism spectrum disorder, neurodegenerative disease, cognitive deficit, mental disorder and cytoskeletal disorder.

According to an aspect of some embodiments of the present invention there is provided a method of monitoring efficacy of treatment in a subject diagnosed with a disease selected from the group consisting of autism spectrum disorder, neurodegenerative disease, cognitive deficit, mental disorder and cytoskeletal disorder, the method comprising determining a level of a marker selected from the group consisting of SMOX, ARRB1, ADCY6, FOXO3, STOM, DNAJB4, TMCC2 and CPXM1 in a biological sample of the subject following the treatment, wherein a decrease in the level of the SMOX, ARRB1, ADCY6, FOXO3, STOM, DNAJB4 and/or TMCC2 and/or an increase in the level of the CPXM1 following the treatment indicates the treatment is efficacious.

According to an aspect of some embodiments of the present invention there is provided a method of monitoring efficacy of treatment in a subject diagnosed with a disease selected from the group consisting of autism spectrum disorder and intellectual disability, the method comprising determining a level of a marker selected from the group consisting of SMOX, ARRB1, ADCY6, FOXO3, STOM, DNAJB4, TMCC2 and CPXM1 in a biological sample of the subject following the treatment, wherein a decrease in the level of the SMOX, ARRB1, ADCY6, FOXO3, STOM, DNAJB4 and/or TMCC2 and/or an increase in the level of the CPXM1 following the treatment indicates the treatment is efficacious.

According to an aspect of some embodiments of the present invention there is provided a method of monitoring efficacy of treatment in a subject diagnosed with Alzheimer's disease, the method comprising determining a level of a marker selected from the group consisting of SMOX, ARRB1, ADCY6, FOXO3, STOM, DNAJB4, TMCC2 and CPXM1 in a biological sample of the subject following the treatment, wherein a decrease in the level of the SMOX, ARRB1, ADCY6, FOXO3, STOM, DNAJB4 and/or TMCC2 and/or an increase in the level of the CPXM1 following the treatment indicates the treatment is efficacious.

According to an aspect of some embodiments of the present invention there is provided a method of diagnosing a disease selected from the group consisting of autism spectrum disorder, neurodegenerative disease, cognitive deficit, mental disorder and cytoskeletal disorder, the method comprising determining a level of a marker selected from the group consisting of SMOX, ARRB1, ADCY6, FOXO3, STOM, DNAJB4, TMCC2 and CPXM1 in a biological sample of the subject, wherein when the level of the SMOX, ARRB1, ADCY6, FOXO3, STOM, DNAJB4 and/or TMCC2 is above a predetermined threshold and/or the level of the CPXM1 is below a predetermined threshold the subject has the disease.

According to an aspect of some embodiments of the present invention there is provided a method of diagnosing a disease selected from the group consisting of autism spectrum disorder and intellectual disability, the method comprising determining a level of a marker selected from the group consisting of SMOX, ARRB1, ADCY6, FOXO3, STOM, DNAJB4, TMCC2 and CPXM1 in a biological sample of the subject, wherein when the level of the SMOX, ARRB1, ADCY6, FOXO3, STOM, DNAJB4 and/or TMCC2 is above a predetermined threshold and/or the level of the CPXM1 is below a predetermined threshold the subject has the disease.

According to an aspect of some embodiments of the present invention there is provided a method of diagnosing Alzheimer's disease, the method comprising determining a level of a marker selected from the group consisting of SMOX, ARRB1, ADCY6, FOXO3, STOM, DNAJB4, TMCC2 and CPXM1 in a biological sample of the subject, wherein when the level of the SMOX, ARRB1, ADCY6, FOXO3, STOM, DNAJB4 and/or TMCC2 is above a predetermined threshold and/or the level of the CPXM1 is below a predetermined threshold the subject has the disease.

According to some embodiments of the invention, the biological sample comprises a blood sample.

According to some embodiments of the invention, the disease is an ADNP syndrome.

According to some embodiments of the invention, the disease is selected from the group consisting of multiple sclerosis, SYNGAP1 syndrome, POGZ syndrome, CHD8 syndrome, SCN2A syndrome, ARID1B-related syndrome, NRXN1 syndrome, DYRK1A syndrome, GRIN disorder, CHD2 syndrome, Dravet syndrome, Rett syndrome, fragile X syndrome, FOXP1 syndrome, SLC-related disorders, Coffin-Siris syndrome, KMT5B syndrome, PTEN autism syndrome, Okihiro syndrome plus developmental delay, Angelman syndrome, Noonan syndrome, Kleefstra syndrome, and Smith-Magenis syndrome.

According to an aspect of some embodiments of the present invention there is provided a method of treating a disease associated with SHANK3 in a subject in need thereof, the method comprising administering to the subject a therapeutically effective amount of an ADNF III polypeptide, wherein the ADNF III polypeptide comprises an SH3 binding domain, thereby treating the disease in the subject.

According to some embodiments of the invention, the disease is Phelan McDermind syndrome.

According to some embodiments of the invention, the ADNF III polypeptide has a neurotrophic/neuroprotective activity in an in vitro cortical neuron culture assay. According to some embodiments of the invention, the treatment comprises an ADNF polypeptide, wherein the ADNF polypeptide has a neurotrophic/neuroprotective activity in an in vitro cortical neuron culture assay.

According to some embodiments of the invention, the ADNF polypeptide is capable of binding EB1 and/or EB3.

According to some embodiments of the invention, the ADNF polypeptide comprises an SH3 binding domain.

According to some embodiments of the invention, the ADNF polypeptide is an ADNF III polypeptide.

According to some embodiments of the invention, the polypeptide comprises an amino acid sequence selected form the group consisting of 2-22.

According to some embodiments of the invention, the polypeptide comprises an amino acid sequence selected form the group consisting of 2-20.

According to some embodiments of the invention, the polypeptide comprises SEQ ID NO: 2.

According to some embodiments of the invention, the polypeptide has the formula (R¹)ₓ-Asn-Ala-Pro-Val-Ser-Ile-Pro-Gln-(R²)_{y} (SEQ ID NO: 49), or an analogue thereof, in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

According to some embodiments of the invention, the ADNF polypeptide is an ADNF I polypeptide.

According to some embodiments of the invention, the polypeptide comprises an amino acid sequence selected form the group consisting of 24-48.

According to some embodiments of the invention, the polypeptide comprises SEQ ID NO: 24.

According to some embodiments of the invention, the polypeptide has the formula (R¹)ₓ-Ser-Ala-Leu-Leu-Arg-Ser-Ile-Pro-Ala-(R²)_{y} (SEQ ID NO: 50), or an analogue thereof, in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

According to some embodiments of the invention, the polypeptide comprises at least one D-amino acid.

According to some embodiments of the invention, the polypeptide is less than 50 amino acids in length.

According to some embodiments of the invention, the polypeptide is less than 20 amino acids in length.

According to some embodiments of the invention, the polypeptide is attached to a cell penetrating or stabilizing moiety.

According to some embodiments of the invention, the subject is a female.

According to some embodiments of the invention, the subject is a male.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### In the Drawings:

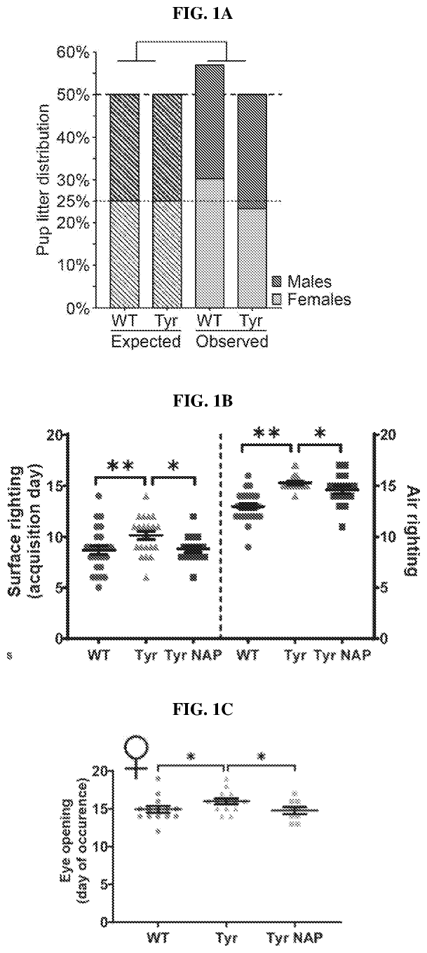

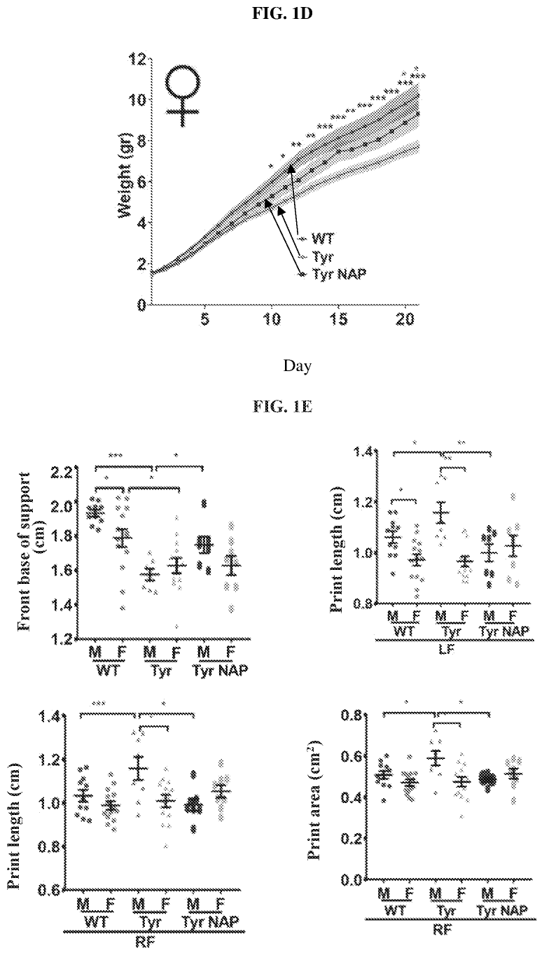

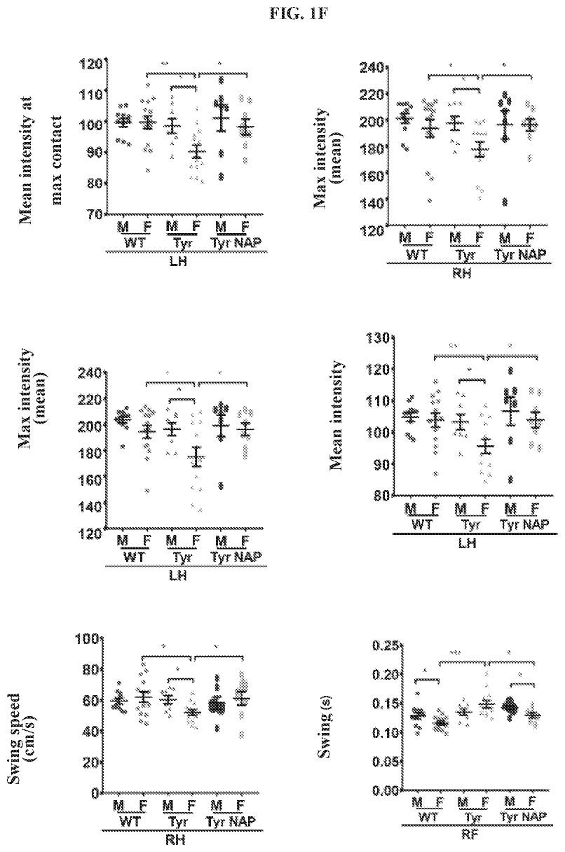

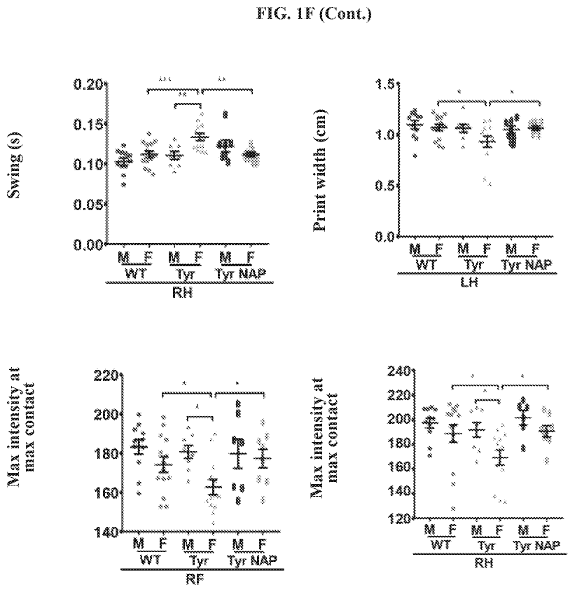

FIGS. 1A-F demonstrate that Tyr mice (genome-edited mice carrying the most prevalent ADNP syndrome mutation, Karmon G, et al. Ibid) are developmentally impaired in a sex-dependent manner, while NAP repairs the phenotype. FIG. 1A shows a two tailed binomial test demonstrating that significantly less Tyr mouse pups survive at postnatal day 1 (Tyr n=128, WT n=178). Expected vs. observed percentages are presented (**P=0.005). Presented male/female ratio is insignificant. FIGS. 1B-C show surface righting, air righting and eye opening (in females), demonstrating significant differences discovered between WT and Tyr littermates with significant NAP amelioration. FIG. 1D demonstrates significant differences between female WT and Tyr pup weight with a beneficial effect with NAP treatment (marked with black and red asterisks respectively). FIG. 1E-F show gait analysis performed on 9.23±0.15-week-old mice. An unpaired Student's t test was also used to determine sex differences. Males: WT n=10, Tyr n=7, Tyr NAP n=7; females: WT n=14, Tyr n=13, Tyr NAP n=8. For swing speed, swing, print width, mean and maximum intensity at maximum contact, maximum and mean intensity significant differences between female WT and Tyr mice were found with significant NAP correction effect. In print area, length and front base of support significant differences between male WT and Tyr mice were found with significant NAP correction effect, with a significant difference between female WT and Tyr mice in front base of support. Sex differences were observed in all gait parameters except print width. RF—right front, RH— right hind, LF—left front, LH—left hind. All data is expressed as mean±SEM. (*P<0.05, **P<0.01, ***P<0.001). A two-way (FIG. 1B-C and 1E-F) or two-way repeated measures (FIG. 1D) ANOVA with Tukey post hoc test was performed unless otherwise stated.

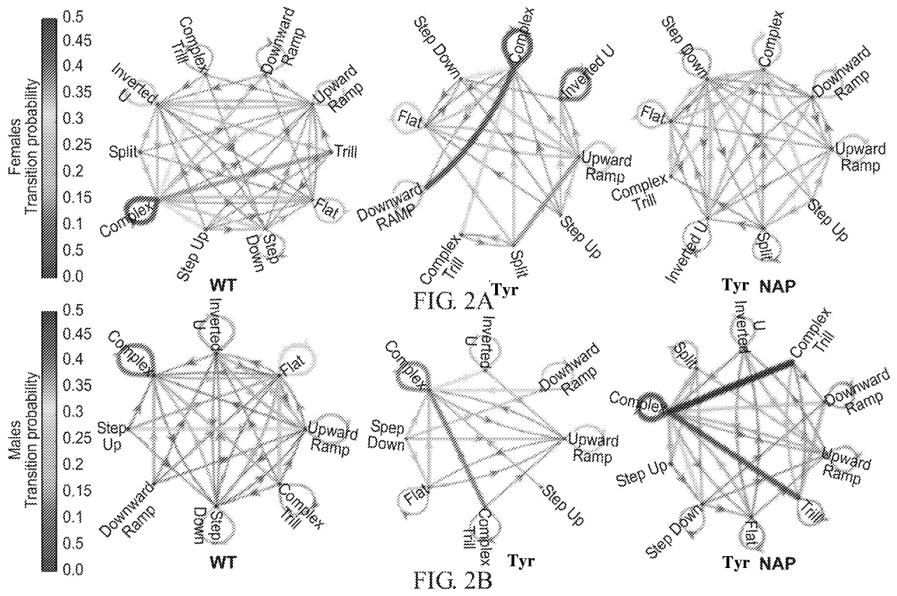

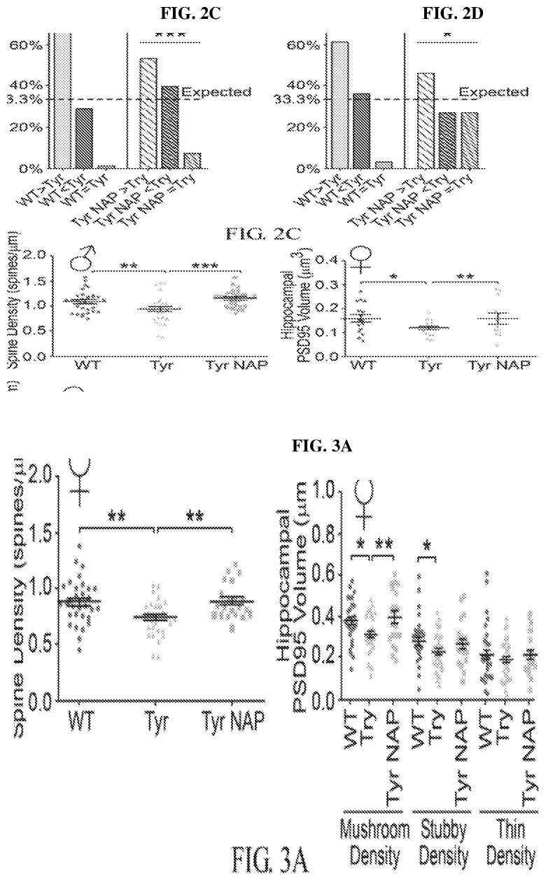

FIGS. 2A-D demonstrate that Tyr mice exhibit impaired syntax in a sex-dependent manner, while NAP repairs the phenotype. A syntax transition probability chart is presented for females (FIG. 2A) and males (FIG. 2B) and was generated using the DeepSqueak algorithm (males: WT n=12, Tyr n=14, Tyr NAP n=10; females: WT n=20, Tyr n=9, Tyr NAP n=9). Syllables measured included: upward ramp, flat, inverted U, complex, step up, downward ramp, step down, complex trill, split and trill. An evident decrease in syntax complexity was observed between WT and Tyr females with a marked improvement with NAP treatment. The same effect can be observed between WT and Tyr males with NAP improvement between Tyr and Tyr NAP male pups. FIG. 2C-D show quantitative graphs in females and males (respectively) in order to quantify this difference, Xi square analysis was performed whereby the transition probabilities between each of the syllables were compared between groups (WT vs. Tyr and Tyr vs. Tyr NAP), the amount of transition probabilities that were larger, smaller or the same between the groups was counted alongside total potential number of transitions. This was analyzed against expected values of: ⅓ of the transition probabilities where one group was better than the other, ⅓ where the reverse occurs and ⅓ where they performed the same. 33.3% (expected) is marked. Xi square analysis revealed that WT female mice performed significantly better than female Tyr mice (***p<0.001) with significant improvement with NAP treatment (***p<0.001). In males the same significant changes were discovered between male WT and Tyr (***p<0.001) and male Tyr and Tyr NAP (*p=0.027).

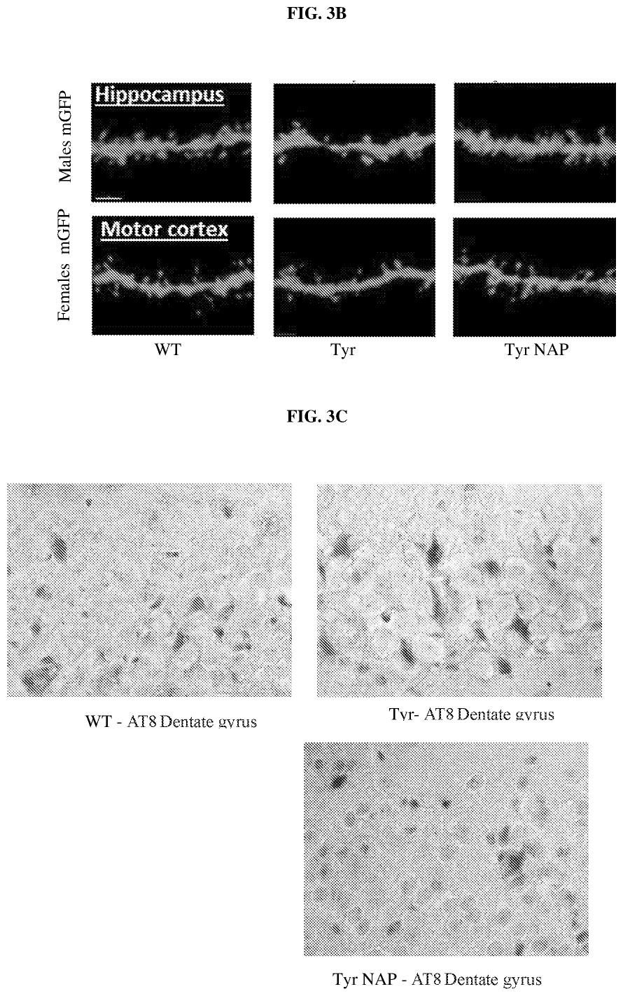

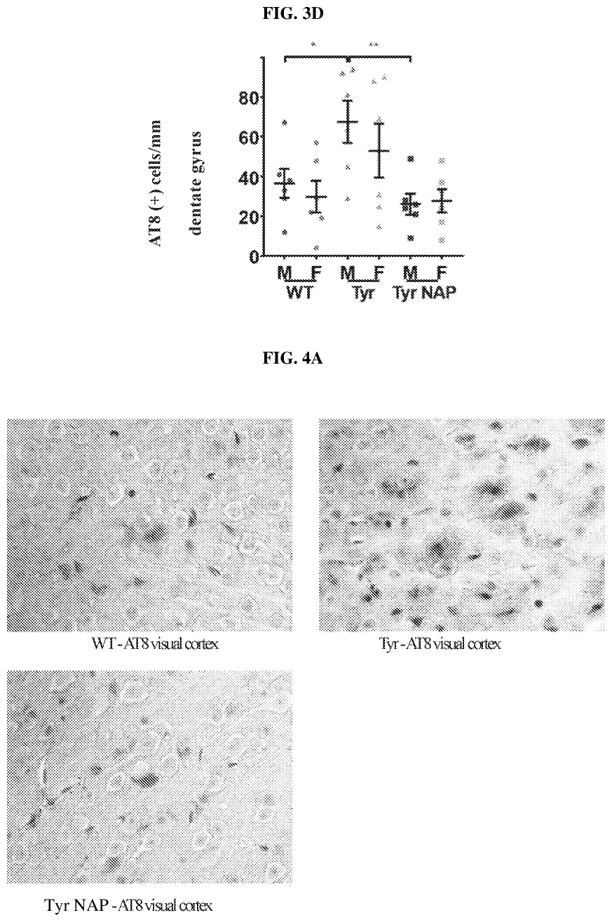

FIGS. 3A-D demonstrate that Tyr mice exhibit reduced dendritic spines, paralleled by tauopathy in a sex-dependent manner, while NAP repairs the phenotype. FIG. 3A shows average total, sub-type spine densities, as well as hippocampal PSD95 volumes (males: WT n=32; Tyr n=32; Tyr NAP, n=31, females: WT n=16-31; Tyr n=16-32; Tyr NAP, n=12-21 dendrites/experimental group), as determined by two-way ANOVA with Tukey post hoc test. Total spine density was significantly decreased in Tyr mice (male hippocampus and female cortex), with NAP significantly increasing it in both brain regions. In males: for hippocampal total spine density, main treatment (F_{(1,123)}=6.876, p=0.010) and interaction (F_{(1,123)}=8.502, p=0.004) effects were found. In females: For PSD95 volume in the hippocampus, a main interaction effect was found (F_{(1,54)}=12.987, p<0.001). For total spine density in the motor cortex, main genotype (F_{(1,112)}=5.811, p=0.018), and treatment (F_{(1,112)}=6.862, p=0.010) effects were found. For mushroom spine density, a main treatment effect was found (F_{(1,112)}=5.827, p=0.017), whereas for stubby spine density, a main interaction effect was found (F_{(1,112)}=4.291, p=0.041). FIG. 3B shows representative images of dendritic spine staining of male hippocampus and female motor cortex. Scale bar: 2 μm. FIG. 3C-D shows AT8 (hyperphosphorylated Tau) stains in the dentate gyrus using a 2-way ANOVA with Tukey's post hoc test. An unpaired Student's t test was also used to determine sex differences. Males: WT n=5, Tyr n=5, Tyr NAP n=5; females: WT n=4, Tyr n=4, Tyr NAP n=4). FIG. 3C shows representative stains from the male dentate gyrus. Scale bar: 100 μm. FIG. 3D is a graph demonstrating quantitation of positive cells/mm². Treatment [F_{(1,23)}=8.014, P=0.01] and interaction [F_{(1,23)}=5.8, P=0.026] effects were found. Turkey post hoc test revealed significant differences between WT and Tyr mice, and between NAP- and vehicle-treated Tyr mice for all the above. Data is expressed as mean±SEM, *P≤0.05, **P<0.01, ***p<0.001.

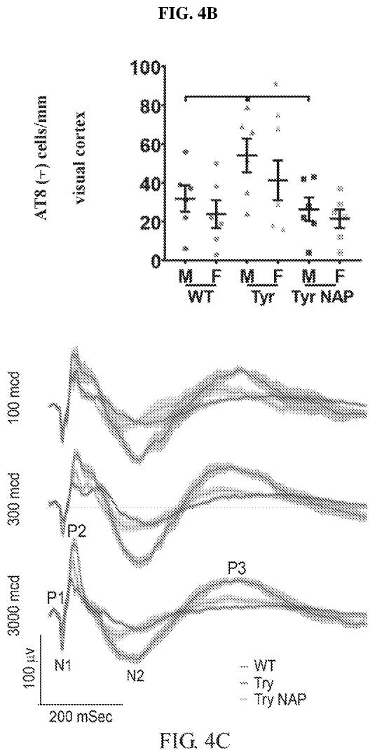

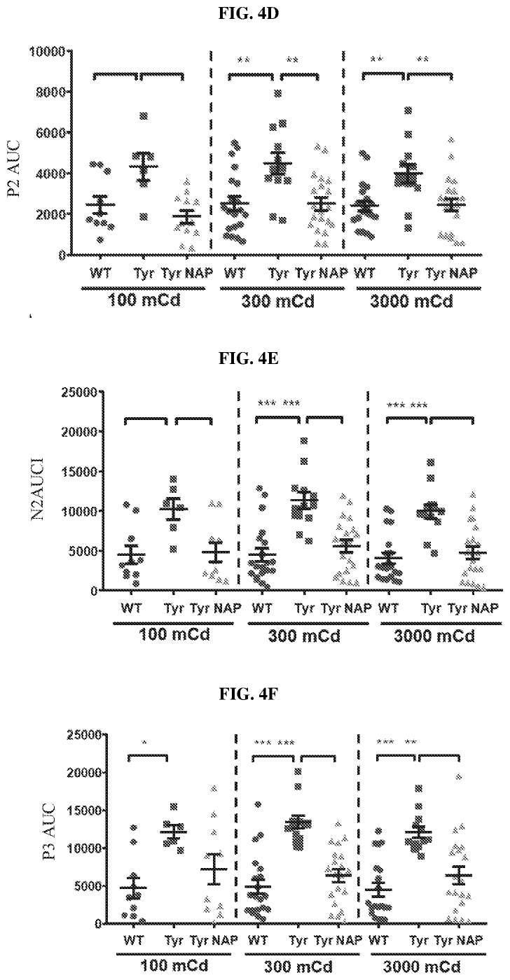

FIGS. 4A-F demonstrate that visual evoked potentials are impaired in male Tyr mice paralleling tauopathy, alongside NAP correction of the phenotype. Tau AT8 antibody immunohistochemical stains were evaluated in the visual cortex using a 2-way ANOVA with Tukey's post hoc test. An unpaired Student's t test was also used to determine sex differences. Males: WT n=5, Tyr n=5, Tyr NAP n=5; females: WT n=4, Tyr n=4, Tyr NAP n=4. FIG. 4A shows representative stains from the male visual cortex. Scale bar: 100 μm. FIG. 4B show quantitation of positive cells/mm². A main treatment effect was found [F_{(1,23)}=5.409, P=0.031]. To assess VEP parameters one-way ANOVA with Tukey post hoc test was performed on male mice. WT N=5, Tyr N=3, Tyr NAP N=5 mice, for 100 mcd WT n=10, Tyr n=6, Tyr NAP n=10, for 300 and 3000 mcd WT n=20, Tyr n=12, Tyr NAP n=20 recordings. FIG. 4C shows average (±SEM) traces for the different stimulation strengths (100, 300 and 3000 mcd respectively), the different wave component nomenclatures are listed on the 3000 mcd traces. FIGS. 4D-F demonstrate that for area under the curve (AUC) of the different wave components, significant differences were discovered between groups. FIG. 4D shows P2 AUC: With 100 mcd stimulation, [F_{(2,25)}=7.179, P=0.003]. With 300 mcd stimulation[F_{(2,51)}=7.448, P=0.001]. With 3000 mcd stimulation [F_{(2,51)}=6.355, P=0.004]. FIG. 4E) shows N2 AUC: With 100 mcd stimulation [F_{(2,25)}=5.752, P=0.009]. With 300 mcd stimulation [F_{(2,51)}=15.090, P<0.001]. With 3000 mcd stimulation [F_{(2,51)}=13.717, P<0.001]. FIG. 4F shows P3 AUC: With 100 mcd stimulation [F_{(2,25)}=4.495, P=0.022]. With 300 mcd stimulation [F_{(2,51)}=20.653, P<0.001]. With 3000 mcd stimulation [F_{(2,51)}=12.598, P<0.001]. Tukey post hoc test revealed significant differences between male Tyr and their WT counterparts with significant NAP amelioration for all of the above except for P3 100 mcd NAP effect. Data is expressed as mean±SEM, *P≤0.05, **P<0.01, ***P<0.001.

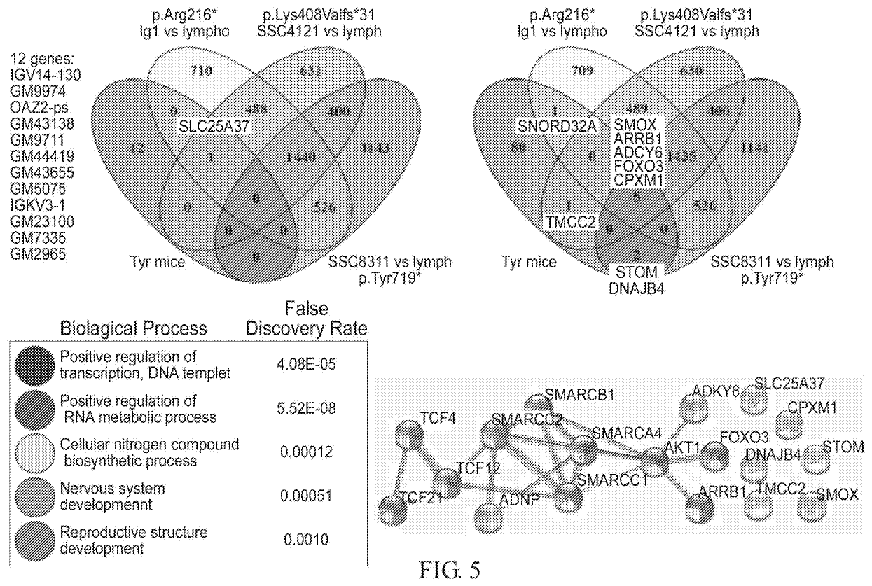

FIG. 5 demonstrates that RNA sequencing of mouse spleens revealed Adnp Tyr genotype-affected/NAP corrected mRNA species that are shared with ADNP mutated human lymphoblastoid cells. RNA sequencing revealed 13 male and 89 female RNA transcripts that were affected by the Tyr Adnp mutation, corrected by NAP treatment affected by three different ADNP mutations including the human equivalent of the Tyr mutation [please see references for example 1 (15, 27)]. Venn diagram (bioinfogp(dot)cnb(dot)csic(dot)es/tools/venny/index2(dot)0(dot)2(dot)html) suggest 1 shared transcript in the males and 9 shared transcripts (including 5 common to all) in females. Further data mining only at the mouse level with more stringent statistics indicated (FDR) indicated significant genotype and NAP effects only in females, with antigen presentation and processing as the main impacted pathway (KEGG pathway analysis, p value <1×10⁻⁸, FDR=3.5×10⁻⁶). Protein-protein interaction analysis (string-db(dot)org/) of the female shared transcripts indicated regulation of neural and sexual development as major affected pathways. As a major interacting ADNP complex is the SWI/SNF chromatin remodeling complex (7) with SMARCA4 (BRG1) as a major interacting protein, was added to the network. Furthermore, as sexual dichotomy is a major characteristic of the Tyr mice, TCF12 and TCF21, major determinant of sexual behavior in humans (56), were also added. Lastly, major shared genes/proteins affected by the ADNP mutations in females including, beta-arrestin-1; forkhead box protein 03 and adenylate cyclase type 6, major cellular regulators were described.

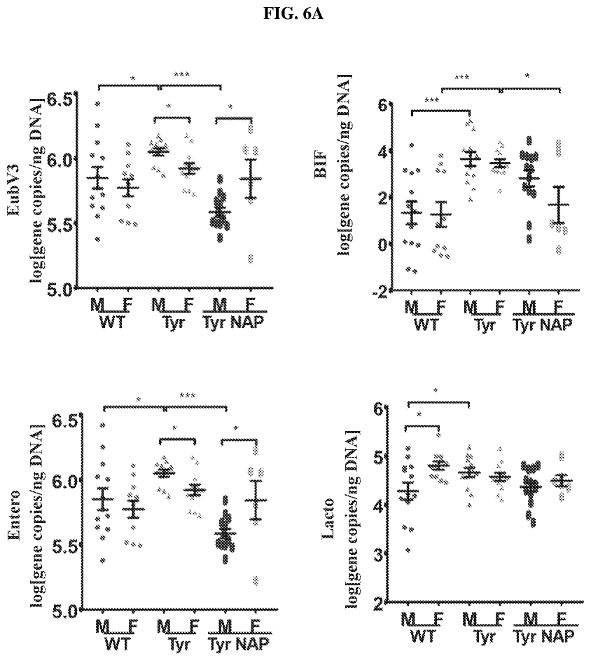

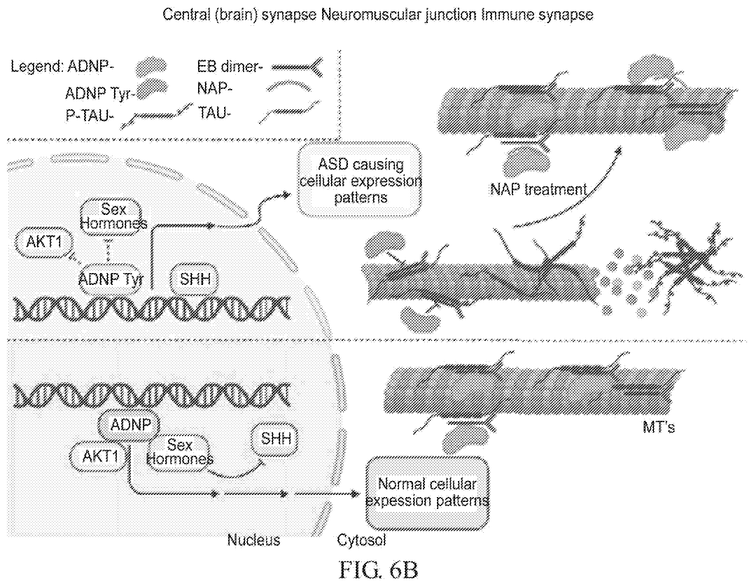

FIGS. 6A-B demonstrate that the Tyr genotype induces gut dysbiosis with NAP correction with a complex, yet explainable, schematic representation of the proposed possible mechanism associated with the immune synapse (57). FIG. 6A demonstrates that fecal microbiota revealed sex and genotype effects, partially ameliorated by NAP treatment. Two-way ANOVA with Tukey post hoc test was performed to assess real-time PCR microbiota loads. An unpaired Student's t test was also used to determine sex differences. Males: WT n=12-13; Tyr n=13; WT NAP n=12; Tyr NAP n=12-13, females: WT n=10-11; Tyr n=12; WT NAP n=11; Tyr NAP n=6. For EubV3, treatment [F_{(1,49)}=15.494, P<0.001] and interaction [F_{(1,49)}=17.752, P<0.001] effects were found in males with no effect in females. For BIF, a main genotype effect [F_{(1,50)}=15.943, P<0.001] was found in males. In females a main interaction effect [F_{(1,39)}=23.1881, P=0.002] was found. For Entero ([F_{(1,47)}=8.378, P=0.006] and Lacto [F_{(1,50)}=4.561, P=0.038], a main genotype effect was found in males, with no effects seen in females. Tukey post hoc test revealed significant differences between male Tyr and their WT littermates in all 4 bacteria, with significant NAP correction of EubV3, as well as significant differences between female Tyr and WT mice with significant NAP amelioration. Sex differences were discovered in the EubV3 and Lacto bacteria. Data is expressed as mean±SEM, *P≤0.05, ***P≤0.001. Total eubacterial load (EubV3), Bifidobacterium genus (BIF), Enterobacteriaceae (Entero), Lactobacillus group (Lacto). FIG. 6B demonstrate a possible proposed mechanism. Based on previous observations (101), recent results linking estrogen, ADNP and SHH (60) and encompassing the results presented herein, a microtubule/Tau centric mechanism is proposed explaining the complex ADNP syndrome phenotype. In the healthy state, microtubule end binding proteins e.g. EB1 is linked with microtubule growing ends, in the neuromuscular junction (102) the immune synapse (103) and the central nervous system (brain) synapse. Created with BioRender(dot)com.

All references cited in FIG. 1A-6B refer to the list of references for Example 1 provided hereinbelow.

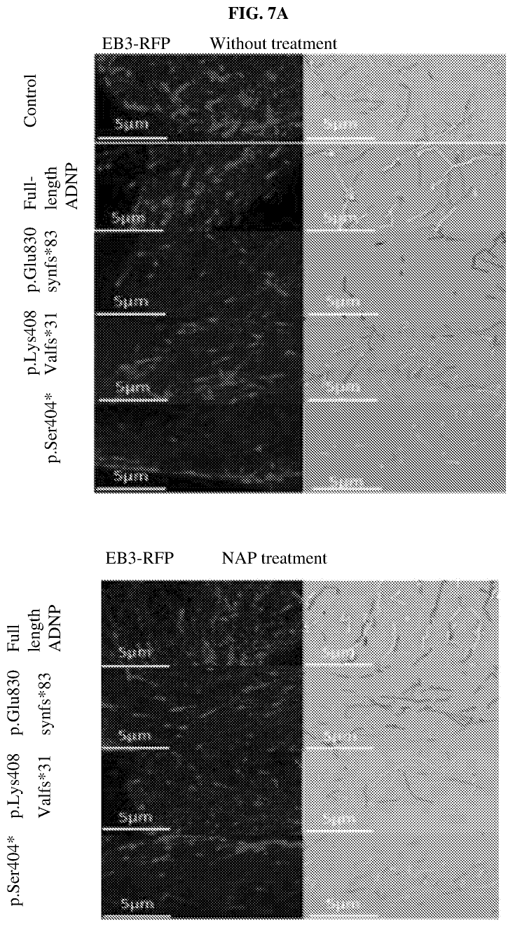

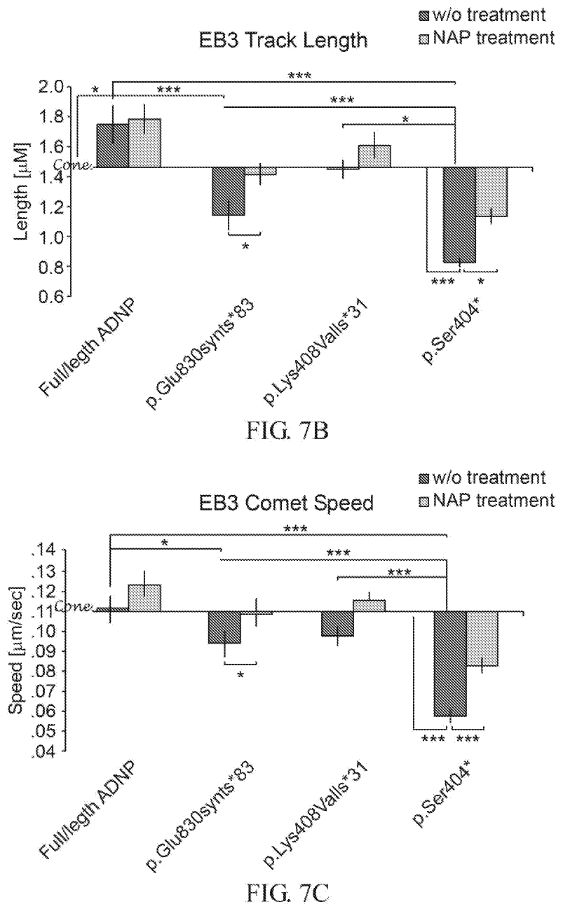

FIGS. 7A-C demonstrate impairment of MT dynamics and assembly by truncated ADNP proteins. FIG. 7A shows live imaging of differentiated NIE-115 cells expressing RFP-tagged EB3 protein and GFP-tagged full-length ADNP or truncated ADNP proteins with or without NAP treatment (10⁻¹² M, 4 hrs). Transfection with backbone plasmid (pEGFP-C1) expressing non-conjugated GFP, was performed as a control. Colored lines (grey squares) represent tracks of EB3 comet-like structures (were obtained by the Imaris software). FIG. 7B shows graphs representing the average (±SEM) of EB3 comet track length and speed. Data was collected from three independent experiments in unbiased fashion by the Imaris software, and statistical analysis of the data was performed by Two Way ANOVA (SigmaPlot 11). Statistical significance *P<0.05, ***P<0.001. Control n=13; full-length ADNP n=14; full-length ADNP+NAP n=14; p.Glu830synfs*83 n=19; p.Glu830synfs*83+NAP n=24; p.Lys408Valfs*31 n=21; p.Lys408Valfs*31+NAP n=20; p.Ser404*n=20; p.Ser404*+NAP n=20.

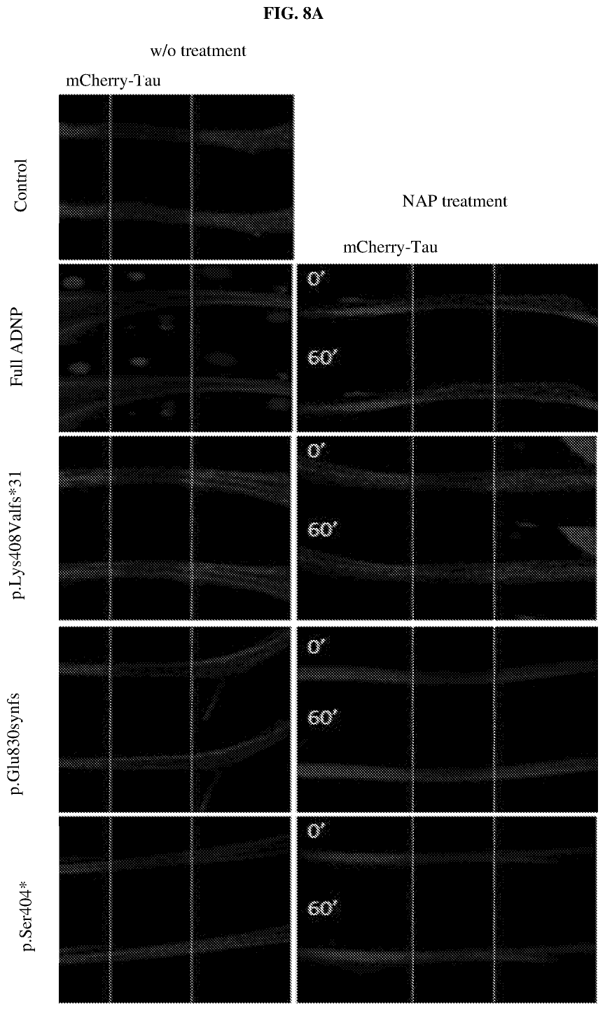

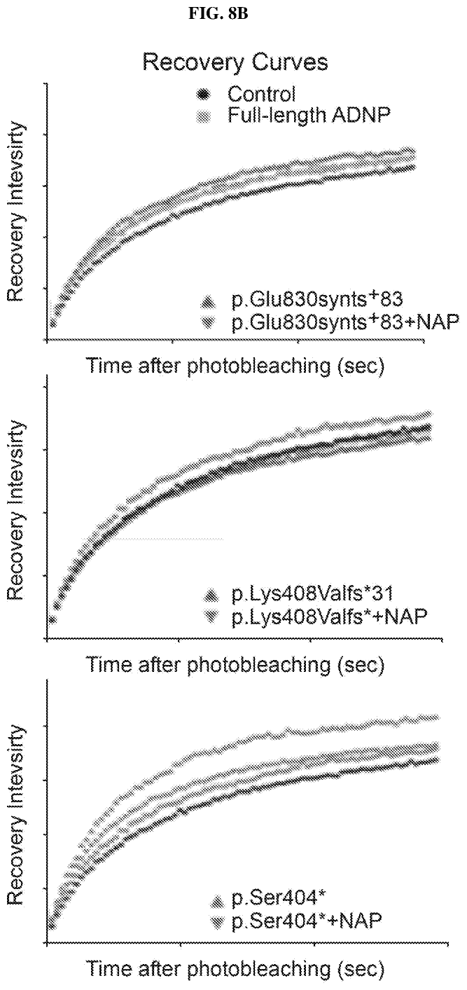

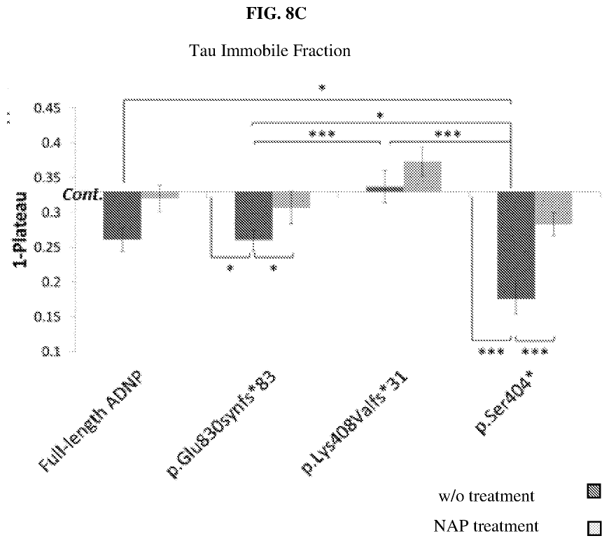

FIGS. 8A-C demonstrate that expression of ADNP truncated proteins deteriorates Tau-MT interaction. FIG. 8A shows representative images of photo-bleaching (0′) and fluorescence recovery (60′) of mCherry-tagged Tau in differentiated N1E-115 cells co-transfected with GFP-tagged full-length ADNP or truncated ADNP proteins with or without NAP treatment (10⁻¹² M, 4 hrs). Transfection with backbone plasmid (pEGFP-C1) expressing non-conjugated GFP, was performed as a control. FIG. 8B shows FRAP recovery curves of normalized data. FIG. 8C is a graph representing averages (±SEM) of the fitted data (from three independent experiments) of immobile fractions. Normalized FRAP data was fitted with one-exponential functions (GraphPad Prism 6) and statistical analysis was done by Two Way ANOVA (SigmaPlot 11). Statistical significance for Control is presented by *P<0.05, **P<0.01, ***P<0.001; significance for full-Length ADNP is presented by $P<0.05 and statistical significance within groups is presented by #P<0.05, ###P<0.001. Control n=64; full-length ADNP n=55; full-length ADNP+NAP n=46; p.Glu830synfs*83 n=71; p.Glu830synfs*83+NAP n=25; p.Lys408Valfs*31 n=36; p.Lys408Valfs*31+NAP n=35; p.Ser404*n=33; p.Ser404*+NAP n=62.

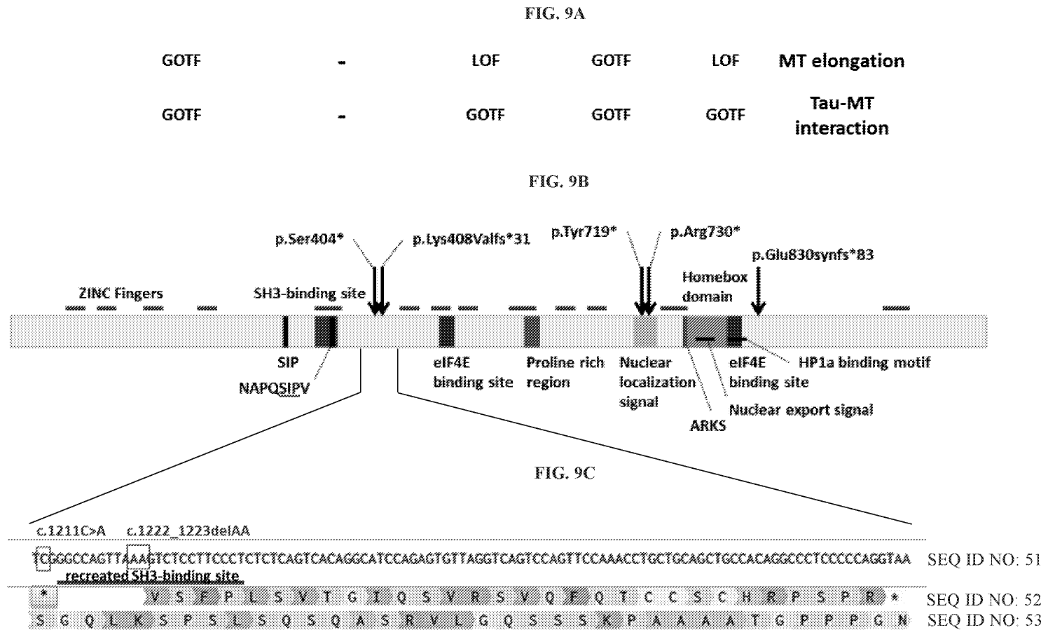

FIG. 9A is a summary of ASD-associated ADNP truncated proteins impact on MT elongation and Tau-MT association, examined, +p.Arg730* and p.Lys719* from previously published data [30, 31]. “LOF”—loss-of-function, “GOTF”—gain-of-toxic-function.

FIG. 9B is a schematic representation demonstrating location of functional protein regions depicted along full-length human ADNP coding sequence. The arrows point out the mutations which have been examined (in the examples part which follows and previously). The figure was constructed according to the previously published findings [30, 31].

FIG. 9C is a schematic representation demonstrating human ADNP cDNA (1210-1316 bp) and amino acid (404-439aa, colored arrows) partial sequences. cDNA sequence represents location of c.1211C<A (p.Ser404*) and c.1222_1223delAA (p.Lys408Valfs*31) mutations. Upper amino acid sequence represents location of stop codons (marked with “*”), frame-shift of 30 amino acids of mutADNP p.Lys4008Valfs*31 and recreated predicted SH3 binding site (red line), lower amino acid sequence—wtADNP. The schematic representation was generated by benchling(dot)com.

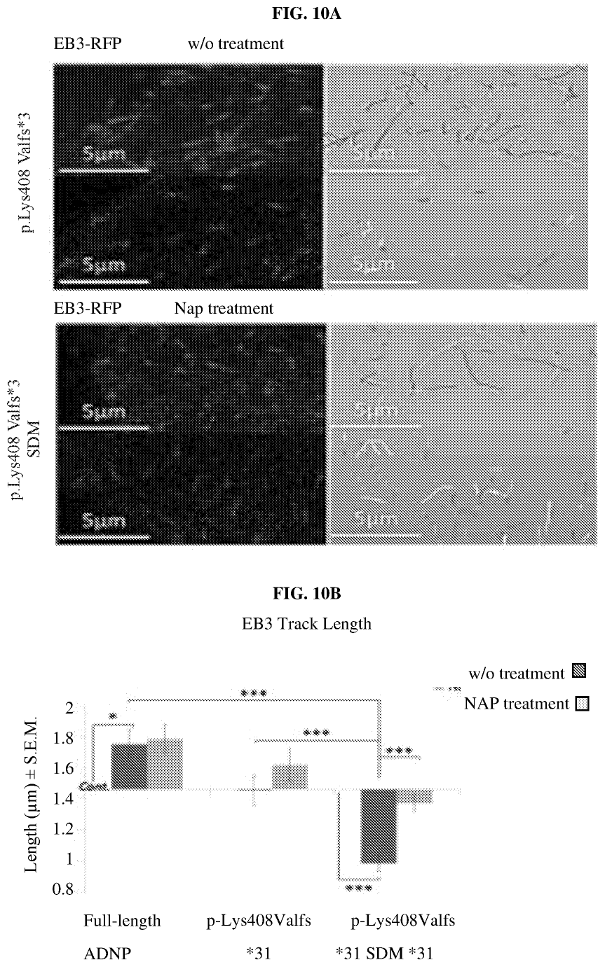

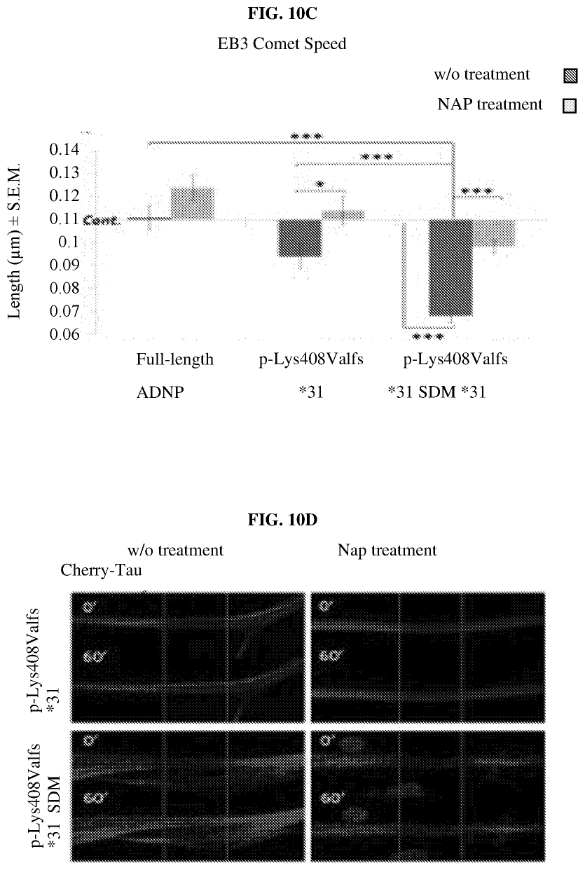

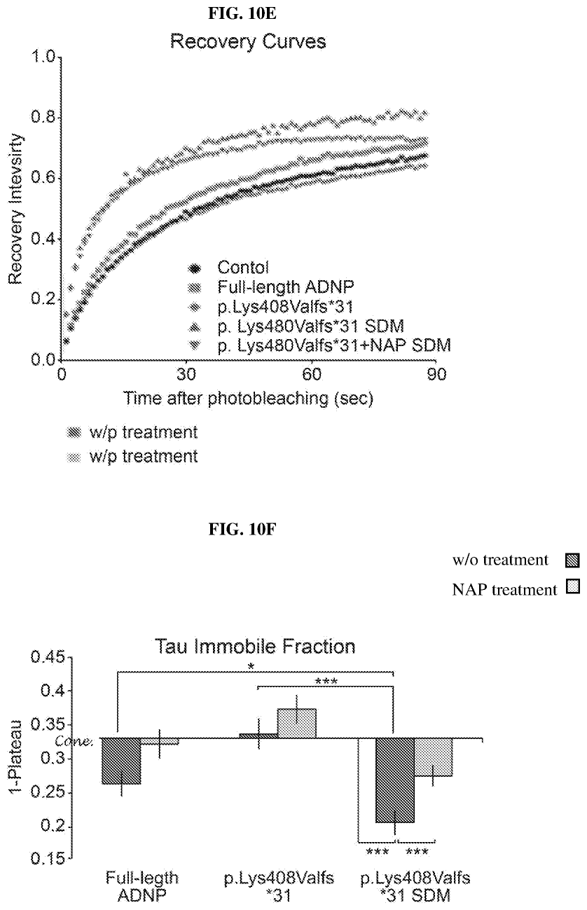

FIGS. 10A-F demonstrate that ADNP p.Lys408Valfs*31 with SDM-destroyed SH3-binding site exhibits adverse effect on MT dynamics and Tau-MT association. FIG. 10A shows time-lapse imaging as described in FIG. 7A hereinabove. Representative images of truncated ADNP p.Lys408Valfs*31 are presented for comparison with p.Lys408Valfs*31 SDM with or without NAP treatment (10⁻¹² M, 4 hrs). FIG. 10B-C show graphs representing the average (±SEM) of EB3 comet track length (FIG. 10B) and speed (FIG. 10C). Data of control, full-length ADNP and p.Lys408Valfs*31 is presented for comparison with p.Lys408Valfs*31 SDM. Statistical analysis was performed as described in FIGS. 7A-B hereinabove. *P<0.05, ***P<0.001; p.Lys408Valfs*31 SDM n=45; p.Lys408Valfs*31 SDM+NAP n=45. FIG. 10D shows FRAP analysis as described in FIG. 8A hereinabove. Representative images of mCherry-tagged Tau photo-bleaching (0′) and fluorescence recovery (60′) of truncated ADNP p.Lys408Valfs*31 presented for comparison with p.Lys408Valfs*31 SDM with or without NAP treatment (10⁻¹² M, 4 hrs). FIG. 10E shows FRAP recovery curves of normalized data. FIG. 10F is a graph representing averages (±SEM) of the fitted data (from three independent experiments) of immobile fractions. Data of control, full-length ADNP and p.Lys408Valfs*31 is presented for comparison with p.Lys408Valfs*31 SDM. Statistical analysis was performed as described in FIGS. 8A-C hereinabove. *P<0.05, ***P<0.001; p.Lys408Valfs*31 SDM n=66; p.Lys408Valfs*31 SDM+NAP n=104.

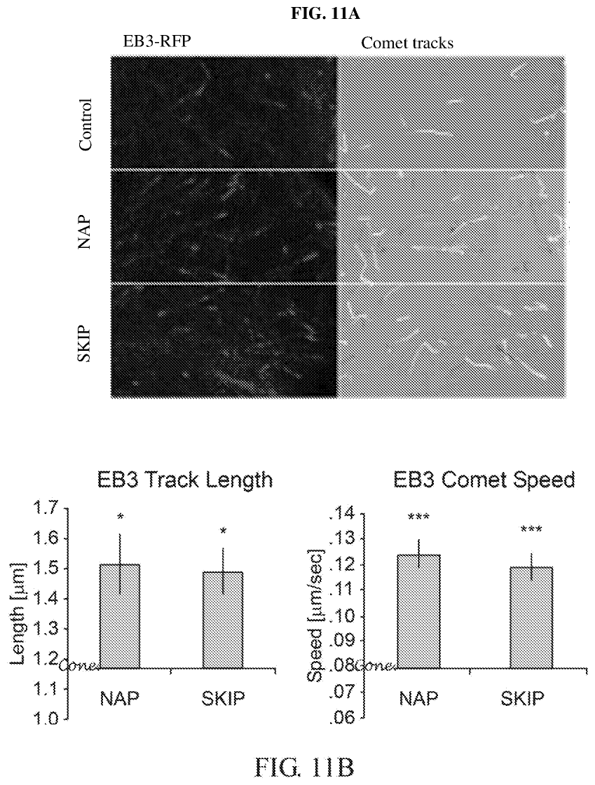

FIGS. 11A-G demonstrate that the SH3 binding domain in NAP increases its protection against tauopathy—i.e. increases Tau-MT association. FIG. 11A shows time-lapse imaging tracking RFP-tagged EB3 protein in differentiated N1E-115 cells with NAP or SKIP treatment (10⁻¹² M, 4 hrs). Control—non-treated cells. Colored lines (grey squares) represent tracks of EB3 comet-like structures (were obtained by the Imaris software). FIG. 11B shows graphs representing the average (±SEM) of EB3 comet track length and speed. Data was collected from three independent experiments in unbiased fashion by the Imaris software, and statistical analysis of the data was performed by One Way ANOVA (IBM SPSS 23). *P<0.05, ***P<0.001; Control n=19, NAP n=16, SKIP n=25. FIG. 11C shows representative images of mCherry-tagged Tau photo-bleaching (0′) and fluorescence recovery (60′) after following treatments of zinc (400 μM, 1 h) with or without NAP/SKIP (10⁻¹² M, 1 h). FIG. 11D is a graph representing averages (±SEM) of the fitted data (from three independent experiments) of immobile fractions. Statistical analysis was performed by One Way ANOVA (IBM SPSS 23). **P<0.01, ***P<0.001; Control n=82, Zn n=43, Zn+NAP n=61, Zn+SKIP n=44. FIG. 11E shows FRAP recovery curves of normalized data. FIG. 11F shows immunoblotting of polymerized (P) and soluble (S) protein fractions (obtained by polymerized vs. soluble tubulin assay) with Tau, tubulin and actin antibodies. Cells were treated with zinc (400 μM, 4 hrs) with or without NAP/SKIP (10⁻¹² M, 4 hrs), non-treated cells were served as control. FIG. 11G shows graphs representing the densitometric quantification of soluble Tau/tubulin ratios. The intensity of each band was quantified by densitometry and the soluble ratio was calculated by dividing the densitometric value of soluble proteins by the total protein content (S/[S+P]). Statistical analysis was performed by One Way ANOVA (IBM SPSS 23). *P<0.05, **P<0.01, ***P<0.001; Tau: control n=15, Zn n=18, Zn+NAP n=12, Zn+SKIP n=9; tubulin: control n=15, Zn n=18, Zn+NAP n=9, Zn+SKIP n=9.

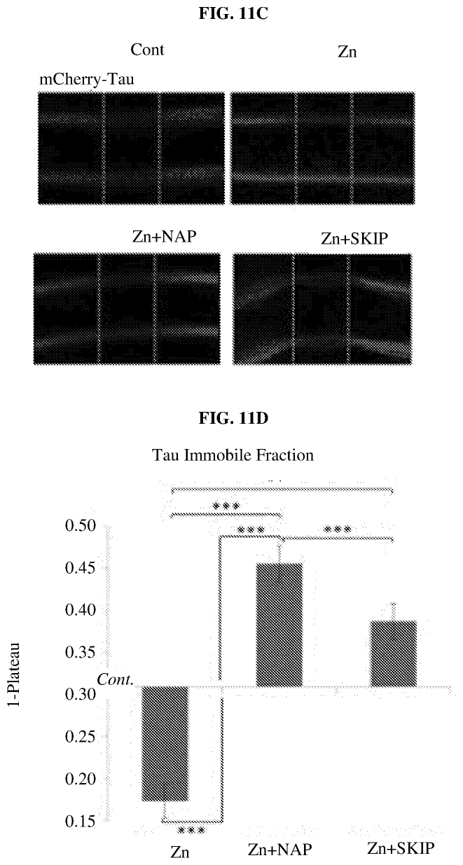

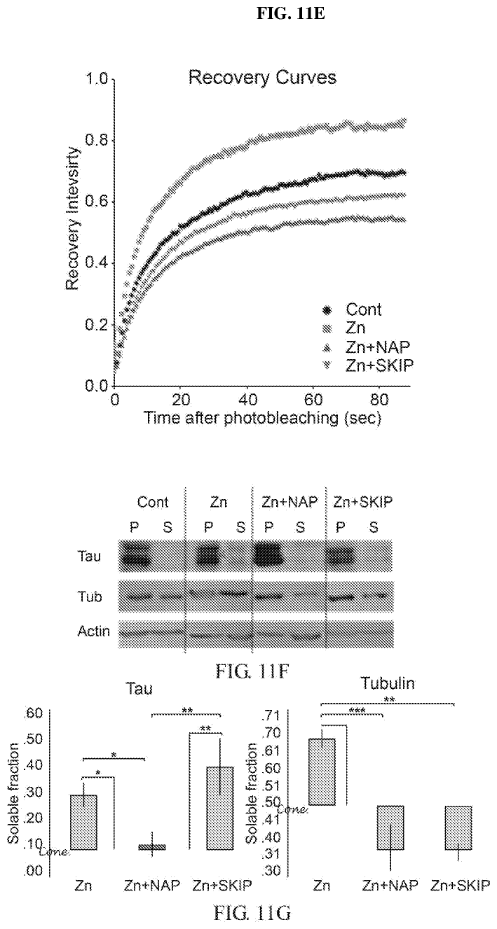

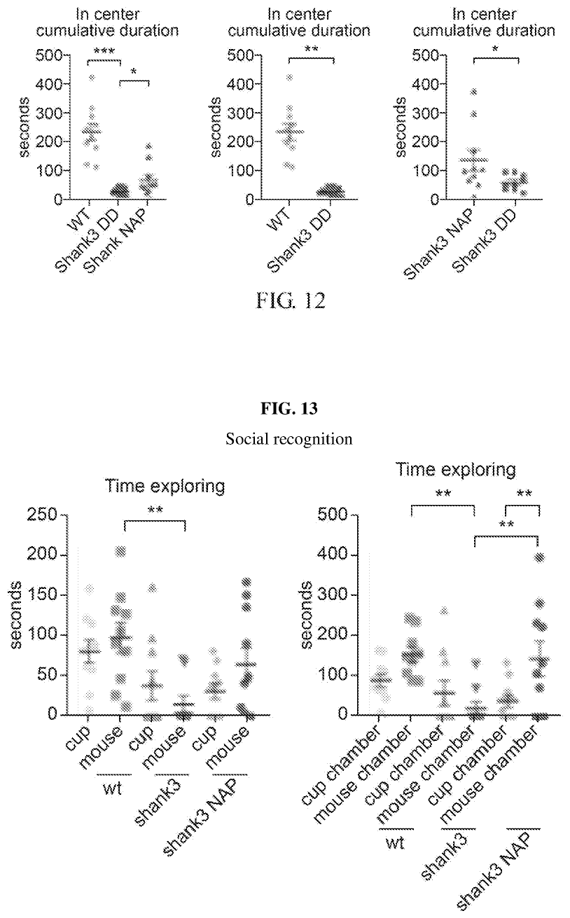

FIG. 12 demonstrates that NAP protects open field behavior against anxiety/depression in the SHANK3 mouse model. *P<0.05; **P<0.01; *P<0.001.

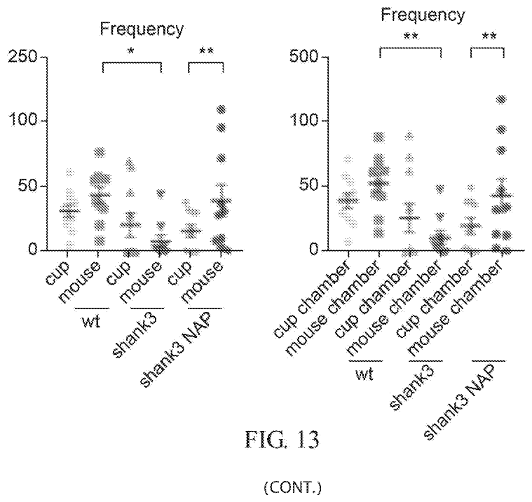

FIG. 13 demonstrates that NAP protects against autistic behavior in the SHANK3 mouse model. *P<0.05; **P<0.01; *P<0.001.

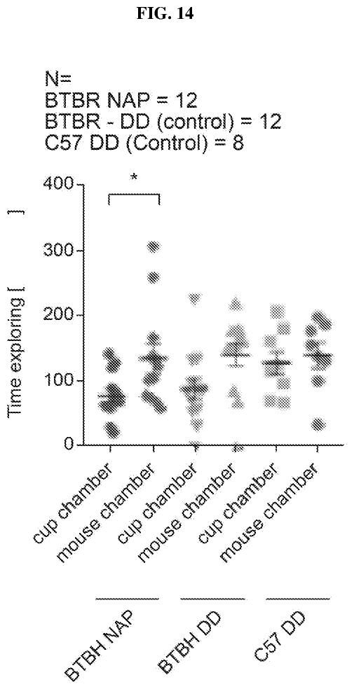

FIG. 14 demonstrate that NAP protects against autistic behavior in the BTBR mouse model. *P<0.05.

### DESCRIPTION OF SPECIFIC EMBODIMENTS OF THE INVENTION

The present invention, in some embodiments thereof, relates to use of ADNF polypeptides in therapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Employing a newly developed murine model of impaired activity-dependent neuroprotective protein (“Tyr” mouse, carrying the NM_001310086 (Adnp_v001):c.2154T>A, p.Tyr718* mutation—see Example 1 of EXAMPLES, below) which reproduces many of the developmental and cognitive anomalies characteristic of the clinically recognized ADNP syndrome, the present inventors have identified a number of novel indications for therapeutic and diagnostic use of ADNF polypeptides.

Thus, in some embodiments there is provided a method of treating a disease in which the subject suffers from visual evoked potential impairment and/or speech impairment that is not due to vocal disturbance, the method comprising administering to the subject a therapeutically effective amount of an ADNF polypeptide, wherein the ADNF polypeptide has a neurotrophic/neuroprotective activity in an in vitro cortical neuron culture assay, thereby treating the disease in the subject.

In some embodiments, the disease is a disease associated with visual evoked potential impairment and/or speech impairment that is not due to vocal disturbance.

In other embodiments, the disease is a disease selected from the group consisting of autism spectrum disorder and intellectual disability.

In still other embodiments, the disease is Alzheimer's disease.

In specific embodiments, the disease is not ADNP syndrome.

In particular embodiments, the disease is a disease associated with visual evoked potential impairment and/or speech impairment that is not due to vocal disturbance, and the disease is not ADNP syndrome. In other embodiments, the disease is selected from the group consisting of autism spectrum disorder and intellectual disability, and the disease is not ADNP syndrome.

The present inventors have shown that administration of an ADNF/ADNP peptide is effective in normalizing aberrant behaviors in the SHANK3-mutated (ASD-linked InsG3680 mutation) mouse model. Thus, according to some aspects of the invention there is provided a method of treating a disease associated with SHANK3 in a subject in need thereof, the method comprising administering to the subject a therapeutically effective amount of an ADNF III polypeptide, wherein said ADNF III polypeptide comprises an SH3 binding domain, thereby treating the disease in the subject.

As used herein the term “SHANK3” [also known as SH3 and multiple ankyrin repeat domains 3 and proline-rich synapse-associated protein 2 (ProSAP2)] refers to the expression product e.g. RNA or protein of the SHANK3 gene (Gene ID 85358). The gene encodes a protein that contains 5 interaction domains or motifs including the ankyrin repeats domain (ANK), a src 3 domain (SH3), a proline-rich domain, a PDZ domain and a sterile a motif domain (SAM).

According to specific embodiments, the SHANK3 is the human SHANK3, such as provided in the following Accession Nos. NM_001080420, NM_001372044, NP_277052.

As used herein the term “disease associated with SHANK3” refers to a disease associated with SHANK3 malfunction (e.g. due to a mutation) for onset and/or progression. A non-limiting example of such a disease is Phelan McDermind syndrome.

As used herein, the term “treating” refers to abrogating, substantially inhibiting, slowing or reversing the progression of a pathology (disease, disorder or condition, e.g. autism spectrum disorder, intellectual disability, visual evoked potential and/or speech impairment, Alzheimer's disease, etc., e.g. in which the subject suffers from visual evoked potential impairment and/or speech impairment that is not due to vocal disturbance), substantially ameliorating a symptom of a pathology and/or improving survival rate in a subject diagnosed with the pathology. Those of skill in the art will understand that various methodologies and assays can be used to assess the development of a pathology or reduction or regression of a pathology, as further disclosed herein.

As used herein, the term “preventing” refers to keeping a pathology from occurring in a subject that has not yet been diagnosed as having the pathology and/or preventing the manifestation of a symptom associated with the pathology before it occurs.

As used herein, the term “improvement” or “improving” refers to reducing or alleviating the severity, frequency or duration of negative aspects of the subject's disease, condition or disorder, or increasing (frequency, strength or duration of) or producing the positive, beneficial or desired aspects of the subject's health and/or well-being associated with the disease, condition or disorder. Some non-limiting examples of improvement following treatment with the ADNF polypeptide according to the invention is improvement in the quality of vocalization, reversal of abnormal evoked potential, shorter time to eye opening and surface and air-righting of “Tyr” mouse pups (see Example 1 of the EXAMPLES section hereinbelow).

As used herein, the phrase “a disease associated with visual evoked potential impairment” refers to a disease wherein a symptom of the disease of a portion of the population having the disease but not all the population is visual evoked potential impairment.

A visual evoked potential (VEP) is an electrical signal generated by the visual cortex in response to visual stimulation. VEPs are elicited by light flashes or by patterned stimuli and recorded by an EEG from occipital electrodes.

As used herein, the term “visual evoked potential impairment” refers to a change in the shape, amplitude and/or response time as compared to a healthy subject.

According to specific embodiments, the visual evoked potential impairment is not due to retinal degeneration.

According to specific embodiments, the subject does not suffer from retinal degeneration.

As used herein, the phrase “a disease associated with speech impairment” refers to a disease wherein a symptom of the disease of a portion of the population having the disease but not all the population is speech impairment.

As used herein, the term “speech impairment” refers to a decrease in speech ability that is not due to vocal disturbance as compared to a healthy subject.

Methods of determining speech ability are known in the art and include, but not limited to, syntax complexity, and are described for example in www(dot)tn(dot)gov/content/dam/tn/education/special-education/eligibility/se_speech_or_language_impairment_evaluation_guidance(dot)pdf; www(dot)asha(dot)org/slp/assessment-and-evaluation-of-speech-language-disorders-in-schools/; www(dot)asha(dot)org/practice-portal/clinical-topics/childhood-apraxia-of-speech/, the contents of which are incorporated herein by references in their entirety. In some embodiments of the invention, the speech impairment or ability is determined according to syntax complexity.

Additional aberrations associated with ADNP insufficiency, which can provide objective measure of related pathology include, but are not limited to auditory brainstem steady state response, electroencephalogram (EEG) and eye tracking. Techniques for measuring auditory brainstem response, typically based on recording of cochlear evoked potential response to auditory stimulus, are well known (see, for example, Stapells and Oates, Audiol and Neuro-Otology 2:257-280, (1997)). In some embodiments, auditory brainstem response is measured as Auditory Steady State Response (Hacohen-Kleiman G, Yizhar-Barnea O, Touloumi O, Lagoudaki R, Avraham K B, Grigoriadis N, Gozes I. Neurochem Res. 2019 June; 44(6):1494-1507).

Eye tracking, an index of visual attention (VA), can be assessed using interactive devices measuring eye movement in response to images and/or text. One currently popular method for measuring eye tracking employs virtual reality headsets fitted with eye movement sensors.

Non-limiting examples of diseases associated with visual evoked potential impairment and/or speech impairment that can be treated according to some embodiments of the invention include neurodegenerative diseases, cognitive deficits, autistic spectrum disorder, mental disorder and cytoskeletal disorders (e.g. Dravet syndrome, Rett syndrome and fragile X syndrome).

As used herein, the term “cognitive deficit” encompasses both intellectual disability and cognitive impairment (typically associated with a mental or neurodegenerative disease).

As used herein the term “intellectual disability (ID)”, also known as general learning disability or mental retardation (MR), refers to a generalized neurodevelopmental disorder characterized by significantly impaired intellectual and adaptive functioning.

Non-limiting examples of neurodegenerative disease or cognitive deficits include, diseases of central motor systems including degenerative conditions affecting the basal ganglia (Huntington's disease, Wilson's disease, striatonigral degeneration, corticobasal ganglionic degeneration), Tourette's syndrome, Parkinson's disease, progressive supranuclear palsy, progressive bulbar palsy, familial spastic paraplegia, spinomuscular atrophy, ALS and variants thereof, dentatorubral atrophy, olivopontocerebellar atrophy, paraneoplastic cerebellar degeneration, and dopamine toxicity; diseases affecting sensory neurons such as Friedreich's ataxia, diabetes, peripheral neuropathy, retinal neuronal degeneration; diseases of limbic and cortical systems such as cerebral amyloidosis, Pick's atrophy, Retts syndrome; neurodegenerative pathologies involving multiple neuronal systems and/or brainstem including Alzheimer's disease, Parkinson's disease, AIDS-related dementia, Leigh's disease, diffuse Lewy body disease, epilepsy, multiple sclerosis, multiple system atrophy, Guillain-Barre syndrome, lysosomal storage disorders such as lipofuscinosis, late-degenerative stages of Down's syndrome, Alper's disease, vertigo as result of CNS degeneration, ALS, corticobasal degeneration, and progressive supranuclear palsy; pathologies associated with developmental retardation and learning impairments, Down's syndrome, fragile X syndrome, Klinefelter's syndrome, Prader-Willi syndrome, cri du chat syndrome and oxidative stress induced neuronal death; pathologies arising with aging and chronic alcohol or drug abuse including, for example, (i) with alcoholism, the degeneration of neurons in locus coeruleus, cerebellum, cholinergic basal forebrain, (ii) with aging, degeneration of cerebellar neurons and cortical neurons leading to cognitive and motor impairments, and (iii) with chronic amphetamine abuse, degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia, closed head trauma, and direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor).

Non-limiting examples of autistic spectrum disorders and intellectual disability include ADNP syndrome, Down's syndrome, SYNGAP1 syndrome, POGZ syndrome, CHD8 syndrome, SCN2A syndrome, ARID1B syndrome, NRXN1 syndrome, DYRK1A syndrome, GRIN disorder, GRIN disorder and CHD2 syndrome.

In some embodiments, additional conditions suitable for treatment, diagnosis or monitoring by the methods of the invention include PTEN autism syndrome, KMT5 syndrome, Okihiro syndrome plus developmental delay, fragile X syndrome, Angelman syndrome, Rett syndrome, Noonan syndrome, Kleefstra syndrome, Smith-Magenis syndrome, and other Coffin-Siris syndrome related disorders.

Non-limiting examples of mental disorders include mood disorders (e.g., major depression disorder (i.e., unipolar disorder), mania, dysphoria, bipolar disorder, dysthymia, cyclothymia), psychotic disorders (e.g., schizophrenia, schizoaffective disorder, schizophreniform disorder, delusional disorder, brief psychotic disorder, and shared psychotic disorder), personality disorders, aggression, anxiety disorders (e.g., obsessive-compulsive disorder and attention deficit disorders) as well as other mental disorders such as substance-related disorders, childhood disorders, dementia, adjustment disorder, delirium, multi-infarct dementia, and Tourette's disorder as described in Diagnostic and Statistical Manual of Mental Disorders, Fourth Edition, (DSM IV) (see also Benitez-King G. et al, Curr Drug Targets CNS Neurol Disord. 2004 December; 3(6):515-33. Review). Typically, such disorders have a complex genetic and/or a biochemical component.

According to specific embodiments, the disease treated by some embodiments of the invention is not an ADNP syndrome.

According to specific embodiments, the disease treated by some embodiments of the invention is an ADNP syndrome.

According to some embodiments of the invention, the disease is selected from the group consisting of multiple sclerosis, SYNGAP1 syndrome, POGZ syndrome, CHD8-related disorder, SCN2A syndrome, ARID1B-related syndrome, NRXN1 syndrome, DYRK1A syndrome, GRIN disorder, CHD2 syndrome, Dravet syndrome, Rett syndrome, fragile X syndrome, GRIN Disorder, POGZ syndrome, FOXP1 syndrome, SLC-related disorders, Coffin-Siris syndrome, KMT5B syndrome, PTEN autism syndrome, Okihiro syndrome plus developmental delay, Angelman syndrome, Noonan syndrome, Kleefstra syndrome, and Smith-Magenis syndrome.

The present inventors have uncovered sex-related differences some, but not all aspects of the “Tyr” mouse phenotype (see, for example, Example 1 of the EXAMPLES section hereinbelow), as well as in the response of the “Tyr” mouse to treatment with ADNF/ADNP peptides. Thus, in some embodiments, the subject is a male. In other embodiments, the subject is a female.

As used herein, the term “activity-dependent neuroprotective factor (ADNF)” refers to ADNF III (also known as ADNP) and/or ADNF I.

As used herein, the term “ADNF polypeptide” refers to the amino acid sequence of human ADNF III and/or ADNF I, or a functional homolog thereof, having at least one of the activities of ADNF III or ADNF I, as further described hereinbelow. According to specific embodiments, the phrase “ADNF polypeptide” refers to a mixture of an ADNF III polypeptide and an ADNF I polypeptide.

As use herein, the phrase “a functional homolog” refers to a fragment, a naturally occurring or synthetically/recombinantly produced homolog, a non-human homolog, an allelic or polymorphic variant, an amino acid sequence comprising conservative and non-conservative amino acid substitutions deletions or additions, an analog, a lipophilic variant and/or a chemically modified variant, which maintains at least one of the activities of the full length protein, e.g. neurotrophic/neuroprotective activity, binding EB1 and/or EB3, binding an SH3 domain, as further described hereinbelow.

As used herein, the term “polypeptide”, “peptide” or “amino acid sequence”, which are interchangeably used herein, encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)—CO—), ester bonds (—C(═O)—O—), ketomethylene bonds (—CO—CH2—), sulfinylmethylene bonds (—S(═O)—CH2—), α-aza bonds (—NH—N(R)—CO—), wherein R is any alkyl (e.g., methyl), amine bonds (—CH2—NH—), sulfide bonds (—CH2—S—), ethylene bonds (—CH2—CH2—), hydroxyethylene bonds (—CH(OH)—CH2—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

According to specific embodiments, the polypeptide comprises at least one D-amino acid.

According to specific embodiments, the polypeptide comprises at least two, at least three, at least 4, at least 5, at least 6, at least 8 D-amino acids.

According to specific embodiments, all the polypeptide amino acids are D-amino acids.

Tables 1 and 2 below list naturally occurring amino acids (Table 2), and non-conventional or modified amino acids (e.g., synthetic, Table 3) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2- | Nnbhm |
| diphenylethyl)glycine | | diphenylethyl)carbamylmethyl- | |
| | | glycine | |
| N-(3,3- | Nbhe | N-(N-(3,3- | Nnbhe |
| diphenylpropyl)glycine | | diphenylpropyl)carbamylmethyl- | |
| | | glycine | |
| 1-carboxy-1-(2,2- | Nmbc | 1,2,3,4-tetrahydroisoquinoline- | Tic |
| diphenylethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | hydroxylysine | |

The amino acids of the polypeptides of some embodiments of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

Conservative substitution tables providing functionally similar amino acids are well known in the art. Guidance concerning which amino acid changes are likely to be phenotypically silent can also be found in Bowie et al., 1990, Science 247: 1306 1310.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)5-COOH]—CO— for aspartic acid. Those non-conservative substitutions which fall under the scope of the present invention are those which still constitute a peptide having neuroprotective properties.

The polypeptides of some embodiments of the invention are preferably utilized in a linear form, although it will be appreciated that in cases where cyclicization does not severely interfere with peptide characteristics, cyclic forms of the peptide can also be utilized.

Since according to specific embodiments, the present polypeptides are utilized in therapeutics which require the peptides to be in soluble form, the polypeptides of some embodiments of the invention include one or more non-natural or natural polar amino acids, including but not limited to serine and threonine which are capable of increasing peptide solubility due to their hydroxyl-containing side chain.

According to specific embodiments, the polypeptide is less than 100, less than 50, less than 20 or less than 10 amino acids in length.

According to specific embodiments, the polypeptide is 4-100, 4-50, 4-40, 4-20, 4-15, 4-10, 4-8 or 8 amino acids in length, each possibility represents a separate embodiment of the present invention.

According to specific embodiments, the polypeptide is at least 4, at least 5, at least 6, at least 7, at least 8 amino acids in length.

According to specific embodiments, the polypeptide is attached, directly or through a spacer or a linker, to a cell penetrating and/or stabilizing moiety. Such moieties are well known in the art and are further described in details hereinbelow.

According to specific embodiments, the N and/or C termini of the polypeptides of some embodiments of the present invention may be protected by functional groups (i.e. end-capping moieties). Examples of such functional groups can be found, for example, in Green et al., “Protective Groups in Organic Chemistry”, (Wiley, 2.sup.nd ed. 1991), Harrison et al., “Compendium of Synthetic Organic Methods”, Vols. 1-8 (John Wiley and Sons, 1971-1996); and Green and Wuts, “Protecting Groups in Organic Synthesis”, John Wiley and Sons, Chapters 5 and 7, 1991, the teachings of which are incorporated herein by reference. Preferred protecting groups are those that increase stability of the polypeptide and/or facilitate transport of the compound attached thereto into a cell, for example, by reducing the hydrophilicity and increasing the lipophilicity of the compounds.

According to specific embodiments, the end-capping comprises an N terminus end-capping.

Representative examples of N-terminus end-capping moieties include, but are not limited to, formyl, acetyl (also denoted herein as “Ac”), stearyl, trifluoroacetyl, benzyl, benzyloxycarbonyl (also denoted herein as “Cbz”), tert-butoxycarbonyl (also denote d herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”).

According to specific embodiments, the N terminus end-capping comprises an Acetyl.

According to specific embodiments, the N terminus end-capping comprises a stearyl (see e.g. Gozes I, et al. Proc Natl Acad Sci USA. 1996 Jan. 9; 93(1): 427-32).

According to specific embodiments, the end-capping comprises a C terminus end-capping.

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus end-capping may be modified to an amide group.

According to specific embodiments, the C terminus end-capping comprises an Amide.

Other end-capping modifications of peptides include replacement of the amine and/or carboxyl with a different moiety, such as hydroxyl, thiol, halide, alkyl, aryl, alkoxy, aryloxy and the like.

According to other specific embodiments of the invention, the polypeptide is attached to a non-proteinaceous moiety.

According to specific embodiments, the polypeptide and the attached non-proteinaceous moiety are covalently attached, directly or through a spacer or a linker.

The phrase “non-proteinaceous moiety” as used herein refers to a molecule not including peptide bonded amino acids that is attached to the above-described polypeptide. According to a specific embodiment the non-proteinaceous is a non-toxic moiety. Exemplary non-proteinaceous moieties which may be used according to the present teachings include, but are not limited to a drug, a chemical, a small molecule, a polynucleotide, a detectable moiety, polyethylene glycol (PEG), Polyvinyl pyrrolidone (PVP), poly(styrene comaleic anhydride) (SMA), and divinyl ether and maleic anhydride copolymer (DIVEMA). According to specific embodiments of the invention, the non-proteinaceous moiety comprises polyethylene glycol (PEG).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation as further described hereinbelow).

Bioconjugation of the peptide amino acid sequence with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldiimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form. In general, the PEG added to the peptide of some embodiments of the present invention should range from a molecular weight (MW) of several hundred Daltons to about 100 kDa (e.g., between 3-30 kDa). Larger MW PEG may be used, but may result in some loss of yield of PEGylated polypeptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson, Bioconjugate Techniques, Academic Press San Diego, Calif. (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

Conveniently, PEG can be attached to a chosen position in the peptide by site-specific mutagenesis as long as the activity of the conjugate is retained. A target for PEGylation could be any Cysteine residue at the N-terminus or the C-terminus of the peptide sequence. Additionally or alternatively, other Cysteine residues can be added to the peptide amino acid sequence (e.g., at the N-terminus or the C-terminus) to thereby serve as a target for PEGylation. Computational analysis may be effected to select a preferred position for mutagenesis without compromising the activity.

Various conjugation chemistries of activated PEG such as PEG-maleimide, PEG-vinylsulfone (VS), PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1:NaH 5:divinyl sulfone 50, at 0.2 gram PEG/mL DCM). PEG-AC is made under argon by reacting a DCM solution of the PEG-OH with acryloyl chloride and triethylamine (molar ratios: OH 1:acryloyl chloride 1.5:triethylamine 2, at 0.2 gram PEG/mL DCM). Such chemical groups can be attached to linearized, 2-arm, 4-arm, or 8-arm PEG molecules.

Resultant conjugated molecules (e.g., PEGylated or PVP-conjugated peptide) are separated, purified and qualified using e.g., high-performance liquid chromatography (HPLC) as well as biological assays.

The polypeptides and compositions of matter of the present invention may be attached (either covalently or non-covalently) to a penetrating moiety.

According to other specific embodiments, the polypeptide is not attached to a heterologous penetrating moiety. Thus, for Example, the ADNF polypeptide NAP (SEQ ID NO: 2) is bioavailable by endocytosis (see e.g. Ivashko-Pachima Y, Gozes I. J Mol Neurosci. 2020 July; 70(7):993-998), thus being a cell penetrating peptide by itself.

As used herein the phrase “penetrating moiety” refers to an agent which enhances translocation of any of the attached polypeptide or composition of matter comprising same across a cell membrane.

According to one embodiment, the penetrating moiety is a peptide and is attached to the polypeptide (either directly or non-directly) via a peptide bond.

Typically, peptide penetrating moieties have an amino acid composition containing either a high relative abundance of positively charged amino acids such as lysine or arginine, or have sequences that contain an alternating pattern of polar/charged amino acids and non-polar, hydrophobic amino acids.

By way of non-limiting example, cell penetrating peptide (CPP) sequences may be used in order to enhance intracellular penetration; however, the disclosure is not so limited, and any suitable penetrating agent may be used, as known by those of skill in the art.

Cell-Penetrating Peptides (CPPs) are short peptides (≤40 amino acids), with the ability to gain access to the interior of almost any cell. They are highly cationic and usually rich in arginine and lysine amino acids. They have the exceptional property of carrying into the cells a wide variety of covalently and noncovalently conjugated cargoes such as proteins, oligonucleotides, and even 200 nm liposomes. Therefore, according to additional exemplary embodiment CPPs can be used to transport the ADNP polypeptide to the interior of cells.

TAT (transcription activator from HIV-1), pAntp (also named penetratin, Drosophila antennapedia homeodomain transcription factor) and VP22 (from Herpes Simplex virus) are non-limiting examples of CPPs that can enter cells in a non-toxic and efficient manner and may be suitable for use with some embodiments of the invention. Protocols for producing CPPs-cargos conjugates and for infecting cells with such conjugates can be found, for example L Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633].

According to another exemplary embodiment the polypeptide may be incorporated into a particulated delivery vehicle, e.g., a liposome, or a nano- or microparticle by any of the known methods in the art [for example, Liposome Technology, Vol. II, Incorporation of Drugs, Proteins, and Genetic Material, CRC Press; Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43].

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. Liposomes can be of different sizes, may contain a low or a high pH and may be of different charge.

The polypeptides of some embodiments of the invention may be synthesized by any techniques that are known to those skilled in the art of peptide synthesis, such as, but not limited to, solid phase and recombinant techniques.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

Large scale peptide synthesis is described by Andersson Biopolymers 2000; 55(3):227-50. Specific embodiments of the present invention contemplate the use of a combined treatment/prophylaxis comprising the polypeptide and a therapeutic agent other than the polypeptides disclosed herein.

Hence, according to specific embodiments, the polypeptides disclosed herein may be provided to the individual with additional active agents to achieve an improved therapeutic or preventive effect as compared to treatment with each agent by itself. Thus, the polypeptide can be administered alone or with other established or experimental therapeutic regimen to treat or prevent diseases associated with evoked potential and/or speech impairment, autism spectrum disorder and intellectual disability, Alzheimer's disease, autism spectrum disorder, neurodegenerative disease, cognitive deficit, mental disorder and cytoskeletal disorder as detailed herein. In such therapy, measures (e.g., dosing and selection of the complementary agent) are taken to minimize or eliminate adverse side effects which may be associated with combination therapies.

Non-limiting examples of ADNF polypeptides that can be used with specific embodiments of the invention are described in detail in e.g. International Patent Application Publication Nos. WO1992/018140, WO9611948, WO 98/35042, WO 0027875, WO 00/53217, WO01/12654, WO 01/92333, WO 2004/080957, WO 2006/099739, WO2007/096859, 2008/08448, WO 2011/021186, WO/2009/026687, WO2010/075635, 2011/083461, WO 2011/099011, WO2013/171595, WO 2017/130190, WO 2004/060309, WO2003022226 and U.S. Pat. Nos. U.S. Pat. Nos. 5,767,240, 6,174,862, 6,613,740 and 8,586,548; herein each incorporated by reference in their entirety; and further hereinbelow.

According to specific embodiments, ADNF is ADNF III.

“ADNF III”, also known as ADNP (activity-dependent neuroprotective protein), refers to the polypeptide encoded by the ADNP gene (Gene ID 23394). According to specific embodiments, ADNF III is human ADNF III. Full length human ADNF III (ADNP) has a predicted molecular weight of 123,562.8 Da (>1000 amino acid residues) and a theoretical pi of about 6.97. The human ADNF III gene is localized to chromosome 20q13.13-13.2, a region associated with cognitive function. Exemplary full-length amino acid and nucleic acid sequences of ADNF III can be found in WO 98/35042, WO 00/27875, U.S. Pat. Nos. 6,613,740 and 6,649,411. According to specific embodiments, ADNF III amino acid sequence comprises SEQ ID NO: 1.

The ADNF III polypeptide described herein possesses at least one of the activities of the full length ADNF III e.g. neurotrophic/neuroprotective activity as measured with in vitro cortical neuron culture assays, binding EB1 and/or EB3, binding an SH3 domain.

Assays for testing neurotrophic/neuroprotective activity are well known in the art and include, but not limited to, in vitro cortical neuron culture assays described by, e.g., Hill et ah, Brain Res. 603:222-233 (1993); Brenneman & Gozes, J. Clin. Invest. 97:2299-2307 (1996), Gozes et al, Proc. Natl. Acad. ScL USA 93, 427-432 (1996).

Assays for testing binding are well known in the art and include, but not limited, to flow cytometry, BiaCore, bio-layer interferometry Blitz® assay, HPLC.

Non-limiting examples of ADNF III polypeptides that can be used with specific embodiments of the invention are provided in Table 3 hereinbelow.

According to specific embodiments, the ADNF III polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology to any of SEQ ID NO: 1-22.

As used herein, “identity” or “sequence identity” refers to global identity, i.e., an identity over the entire amino acid or nucleic acid sequences disclosed herein and not over portions thereof.

Sequence identity or homology can be determined using any protein or nucleic acid sequence alignment algorithm such as Blast, ClustalW, and MUSCLE.

According to specific embodiments, ADNF is ADNF I.

“ADNF I” refers to the activity dependent neurotrophic factor described in Gozes I, Brenneman D E. J Mol Neurosci. 1996 Winter; 7(4):235-44; Brenneman D E, Gozes I. J Clin Invest. 1996 May 15; 97(10):2299-307 and Brenneman D E, et al. J Pharmacol Exp Ther. 1998 May; 285(2):619-27, the contents of each are incorporated herein by reference in their entirety. According to specific embodiments, ADNF I is human ADNF I. Full length human ADNF I has a predicted molecular weight of about 14,000 Da with a pi of 8.3±0.25. According to specific embodiments, ADNF I amino acid sequence comprises any of SEQ ID NO: 24 or 45.

The ADNF I polypeptide described herein possesses at least one of the activities of the full length ADNF I e.g. neurotrophic/neuroprotective activity as measured with in vitro cortical neuron culture assays, binding EB1 and EB3.

Non-limiting examples of ADNF I polypeptides that can be used with specific embodiments of the invention are provided in Table 3 hereinbelow.

According to specific embodiments, the ADNF I polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology to any of SEQ ID NO: 24-48.

TABLE 3
list of possible ADNF polypeptides that can be used with specific embodiments
of the invention.

| SEQ | ADNF III polypeptide | | SEQ | ADNF I polypeptide | |
|---|---|---|---|---|---|
| ID | Single letter | Three letters | ID | Single letter | Three letters |
| NO: | aa code | aa code | NO: | aa code | aa code |
| 1 | Full length | | 24 | SALLRSIPA | Ser-Ala-Leu-Leu- |
| | | | | | Arg-Ser-Ile-Pro- |
| | | | | | Ala |
| 2 | NAPVSIPQ | Asn-Ala-Pro- | 25 | VAGGGSALLRSI | Val-Ala-Gly-Gly- |
| | | Val-Ser-Ile-Pro- | | PA | Gly-Ser-Ala-Leu- |
| | | Gln | | | Leu-Arg-Ser-Ile- |
| | | | | | Pro-Ala |
| 3 | SVRLGLGGN | Ser-Val-Arg- | 26 | VEEGIVLGGGS | Val-Glu-Glu-Gly- |
| | APVSIPQQS | Leu-Gly-Leu- | | ALLRSIPA | Ile-Val-Leu-Gly- |
| | | Gly-Gly-Asn- | | | Gly-Gly-Ser-Ala- |
| | | Ala-Pro-Val- | | | Leu-Leu-Arg-Ser- |
| | | Ser-Ile-Pro-Gln- | | | Ile-Pro-Ala |
| | | Gln-Ser | | | |
| 4 | LGLGGNAPVS | Leu-Gly-Leu- | 27 | VLGGGCALLR | Val-Leu-Gly-Gly- |
| | IPQQS | Gly-Gly-Asn- | | CIPA | Gly-Cys-Ala-Leu- |
| | | Ala-Pro-Val- | | | Leu-Arg-Cys-Ile- |
| | | Ser-Ile-Pro-Gln- | | | Pro-Ala |
| | | Gln-Ser | | | |
| 5 | LGGNAPVSIP | Leu-Gly-Gly- | 28 | ALLRSIPA | Ala-Leu-Leu- |
| | QQS | Asn-Ala-Pro- | | | Arg-Ser-Ile-Pro- |
| | | Val-Ser-Ile-Pro- | | | Ala |
| | | Gln-Gln-Ser | | | |
| 6 | GGNAPVSIPQ | Gly-Gly-Asn- | 29 | GSALLRSIPA | Gly-Ser-Ala-Leu- |
| | | Ala-Pro-Val- | | | Leu-Arg-Ser-Ile- |
| | | Ser-Ile-Pro-Gln | | | Pro-Ala |
| 7 | Ac- | Ac-Asn-Ala- | 30 | SALLRS | Ser-Ala-Leu-Leu- |
| | NAPVSKIPQ | Pro-Val-Ser-Ile- | | | Arg-Ser |
| | | Pro-Gln | | | |
| 8 | NAPVSKIPQ | Asn-Ala-Pro- | 31 | GSALLRSIPA | Gly-Ser-Ala-Leu- |
| | | Val-Ser-Lys-Ile- | | | Leu-Arg-Ser-Ile- |
| | | Pro-Gln | | | Pro-Ala |
| 9 | NAPBSAIPQ | Asn-Ala-Pro- | 32 | VAGGGSALLRS | Val-Ala-Gly-Gly- |
| | | Asx-Ser-Ala- | | I | Gly-Ser-Ala-Leu- |
| | | Ile-Pro-Gln | | | Leu-Arg-Ser-Ile |
| 10 | NAPVSRIPQ | Asn-Ala-Pro- | 33 | VLGGGSALLR | Val-Leu-Gly-Gly- |
| | | Val-Ser-Arg-Ile- | | | Gly-Ser-Ala-Leu- |
| | | Pro-Gln | | | Leu-Arg |
| 11 | NAPVTRIPQ | Asn-Ala-Pro- | 34 | VLGGGSALL | Val-Leu-Gly-Gly- |
| | | Val-Thr-Arg- | | | Gly-Ser-Ala-Leu- |
| | | Ile-Pro-Gln | | | Leu |
| 12 | NAPVAAAAQ | Asn-Ala-Pro- | 35 | VAGGGSAL | Val-Ala-Gly-Gly- |
| | | Val-Ala-Ala- | | | Gly-Ser-Ala-Leu |
| | | Ala-Ala-Gln | | | |
| 13 | All d-amino | All d-amino | 36 | All d-amino | All d-amino acid |
| | acid | acid Asn-Ala- | | acid | Ser-Ala-Leu-Leu- |
| | NAPVSIPQ | Pro-Val-Ser-Ile- | | SALLRSIPA | Arg-Ser-Ile-Pro- |
| | | Pro-Gln | | | Ala |
| 14 | AAPVSIPQ | Ala-Ala-Pro- | 37 | AALLRSIPA | Ala-Ala-Leu-Leu- |
| | | Val-Ser-Ile-Pro- | | | Arg-Ser-Ile-Pro- |
| | | Gln | | | Ala |
| 15 | NAAVSIPQ | Asn-Ala-Ala- | 38 | SAALRSIPA | Ser-Ala-Ala-Leu- |
| | | Val-Ser-Ile-Pro- | | | Arg-Ser-Ile-Pro- |
| | | Gln | | | Ala |
| 16 | NAPASIPQ | Asn-Ala-Pro- | 39 | SALARSIPA | Ser-Ala-Leu-Ala- |
| | | Ala-Ser-Ile-Pro- | | | Arg-Ser-Ile-Pro- |
| | | Gln | | | Ala |
| 17 | NAPVAIPQ | Asn-Ala-Pro- | 40 | SALLASIPA | Ser-Ala-Leu-Leu- |
| | | Val-Ala-Ile-Pro- | | | Ala-Ser-Ile-Pro- |
| | | Gln | | | Ala |
| 18 | NAPVSAPQ | Asn-Ala-Pro- | 41 | SALLRAIPA | Ser-Ala-Leu-Leu- |
| | | Val-Ser-Ala- | | | Arg-Ala-Ile-Pro- |
| | | Pro-Gln | | | Ala |
| 19 | NAPVSIAQ | Asn-Ala-Pro- | 42 | SALLRSAPA | Ser-Ala-Leu-Leu- |
| | | Val-Ser-Ile-Ala- | | | Arg-Ser-Ala-Pro- |
| | | Gln | | | Ala |
| 20 | NAPVSIPA | Asn-Ala-Pro- | 43 | SALLRSIAA | Ser-Ala-Leu-Leu- |
| | | Val-Ser-Ile-Pro- | | | Arg-Ser-Ile-Ala- |
| | | Ala | | | Ala |
| 21 | SKIP | Ser-Lys-Ile-Pro | 44 | SALLRSIPAPAG | Ser-Ala-Leu-Leu- |
| | | | | ASRLLLLTGEID | Arg-Ser-Ile-Pro- |
| | | | | LP | Ala-Pro-Ala-Gly- |
| | | | | | Ala-Ser-Arg-Leu- |
| | | | | | Leu-Leu-Leu- |
| | | | | | Thr- |
| | | | | | Gly-Glu-Ile-Asp- |
| | | | | | Leu-Pro |
| 22 | Ac-SKIP-NH2 | Ac-Ser-Lys-Ile- | 45 | VLGGGSALLRS | Val-Leu-Gly-Gly- |
| | | Pro-NH2 | | IPA | Gly-Ser-Ala-Leu- |
| | | | | | Leu-Arg-Ser-Ile- |
| | | | | | Pro-Ala |
| | | | 46 | LGGGSALLRSIP | Leu-Gly-Gly-Gly- |
| | | | | A | Ser-Ala-Leu-Leu- |
| | | | | | Arg-Ser-Ile-Pro- |
| | | | | | Ala |
| | | | 47 | GGGSALLRSIP | Gly-Gly-Gly-Ser- |
| | | | | A | Ala-Leu-Leu- |
| | | | | | Arg-Ser-Ile-Pro- |
| | | | | | Ala |
| | | | 48 | GGSALLRSIPA | Gly-Gly-Ser-Ala- |
| | | | | | Leu-Leu-Arg-Ser- |
| | | | | | Ile-Pro-Ala |

In further aspects, the polypeptide comprises an active core site comprising the amino acid sequence of NAPVSIPQ (SEQ ID NO: 2) or SALLRSIPA (SEQ ID NO:24), or conservatively modified variants (e.g., deletion, addition, or substitutions of one or more amino acids) or chemically modified variants thereof, that have neurotrophic/neuroprotective activity as measured with in vitro cortical neuron culture assays as described. An ADNF polypeptide can be derived from an ADNF I polypeptide, an ADNF III polypeptide, their alleles, polymorphic variants, analogs, interspecies homolog, any subsequences thereof or lipophilic variants that exhibit neuroprotective/neurotrophic action on, e.g., neurons originating in the central nervous system either in vitro or in vivo. An ADNF-related neuroprotective peptide can range from as short as four to eight amino acids and can have, e.g., between 8-20, 8-50, 10-100, or about 200, 500, or more amino acids. One non-limiting example of a variant ADNP-related neuroprotective peptide is a 4-amino acid peptide of SKIP (SEQ ID NO: 21), see Amram et al. Sexual Divergence in Microtubule Function: The Novel Intranasal Microtubule Targeting SKIP Normalizes Axonal Transport and Enhances Memory. Mol Psychiatry, 2016; 21:1467-76. Further examples include, but are not limited to all D-amino acid derivatives of NAPVSIPQ (SEQ ID NO: 13) and SALLRSIPA (SEQ ID NO:36).

Thus, according to further aspects of the invention, the polypeptide has the formula (R¹)ₓ-Asn-Ala-Pro-Val-Ser-Ile-Pro-Gln-(R²)_{y} (SEQ ID NO: 49), in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one. In further embodiments, the core amino acid sequence “Asn-Ala-Pro-Val-Ser-Ile-Pro-Gln” of SEQ ID NO: 49 (“Asn-Ala-Pro-Val-Ser-Ile-Pro-Gln” is identical to SEQ ID NO: 2) is replaced by an analogue of SEQ ID NO: 2.

In further aspects of the invention, the polypeptide has the formula (R¹)ₓ-Ser-Ala-Leu-Leu-Arg-Ser-Ile-Pro-Ala-(R²)_{y} (SEQ ID NO: 50), in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one. In further embodiments, the amino acid sequence “Ser-Ala-Leu-Leu-Arg-Ser-Ile-Pro-Ala” of SEQ ID NO: 50 (“Ser-Ala-Leu-Leu-Arg-Ser-Ile-Pro-Ala” is identical to SEQ ID NO: 24) is replaced by an analogue of SEQ ID NO: 24.

Inasmuch as the ADNP peptides and polypeptides of the invention are effective in treating disease in a subject in need thereof, they can also be used for monitoring the efficacy of treatment with ADNF peptides and polypeptides. Thus, in some embodiments there is provided a method of monitoring efficacy of treatment with an ADNF polypeptide in a subject diagnosed with a disease in which the subject suffers from visual evoked potential impairment and/or speech impairment, wherein the subject has a visual evoked potential impairment and/or a speech impairment that is not due to vocal disturbance, comprising determining a visual evoked potential and/or speech ability of the subject following the treatment with the ADNF polypeptide, wherein an improvement in the visual evoked potential and/or said speech ability following the treatment with the ADNF polypeptide indicates the treatment is efficacious.

In some embodiments, the subject is diagnosed with a disease associated with visual evoked potential impairment and/or speech impairment that is not due to vocal disturbance. In other embodiments, the subject is diagnosed with an autism spectrum disorder or intellectual disability. In still further embodiments, the subject is diagnosed with Alzheimer's disease.

According to specific embodiments, the subject is diagnosed with a disease selected from the group consisting of autism spectrum disorder, neurodegenerative disease, cognitive deficit, mental disorder and cytoskeletal disorder.

It will be appreciated that repeat determination of visual evoked potential and/or said speech ability, and comparison with values from healthy individuals not diagnosed with a disease associated with visual evoked potential impairment and/or speech impairment that is not due to vocal disturbance, an autism spectrum disorder or intellectual disability, Alzheimer's disease, neurodegenerative disease, cognitive deficit, mental disorder and cytoskeletal disorder can be performed more often than once, and that multiple assessments of values of the subject's visual evoked potential and/or said speech ability from different time points following treatment with the ADNF polypeptide can be beneficial in determining efficacy, as well as aiding in strategizing the further treatment regimen—e.g. dosage and frequency of administration.

As used herein, the term “monitoring” a subject's visual evoked potential and/or said speech ability generally refers to monitoring, and in particular, recording changes in a subject's condition (e.g. disease-related parameters, for example but not exclusively, change in the shape, amplitude and/or visual response time or speech patterns and vocalizations) e.g. to inform a diagnosis of a disease associated with visual evoked potential impairment and/or speech impairment that is not due to vocal disturbance, an autism spectrum disorder or intellectual disability, Alzheimer's disease, neurodegenerative disease, cognitive deficit, mental disorder and cytoskeletal disorder; to inform a prognosis for a disease associated with visual evoked potential impairment and/or speech impairment that is not due to vocal disturbance, an autism spectrum disorder or intellectual disability, Alzheimer's disease, neurodegenerative disease, cognitive deficit, mental disorder and cytoskeletal disorder, to provide information as to the effect or efficacy of a treatment with ADNF or ADNP peptide or polypeptide, and the like. For example, wherein monitoring efficacy of treatment of the subject indicates a decrease in effect of current treatment, e.g. for intellectual disability or Alzheimer's disease, cognitive deficit and/or cytoskeletal disorder, increased intensity (dosage, frequency, etc) of the treatment, or different treatment options may be considered. Repeated monitoring, as described, can be performed throughout duration of the treatment(s).

Using the “Tyr” mouse model, the inventors have identified biomarkers whose expression is deregulated in correlation with the aberrant phenotype characteristic of the modification (e.g. mutations) of the ADNF gene. Of particular significance were the gene transcripts SMOX (Spermine Oxidase), ARRB1 (Arrestin Beta 1), ADCY6 (Adenylate Cyclase 6), FOXO3 (Forkhead Box 03), and CPXM1 (Carboxypeptidase X, M14 Family Member 1), (STOM (Stomatin), DNAJB4 (DnaJ Heat Shock Protein Family (Hsp40) Member B4)) and TMCC2 (Transmembrane And Coiled-Coil Domain Family 2) (see Example 1 in EXAMPLES below).

As used herein the term “SMOX” [also known as SMO, C20orf16, Polyamine Oxidase, PAO-1, PAOH1, PAOH, DJ779E11.1] refers to the expression product e.g. RNA or protein of the SMOX gene (Gene ID 54498). The gene encodes an FAD-containing enzyme that catalyzes the oxidation of spermine to spermadine and secondarily produces hydrogen peroxide.

According to specific embodiments, the SMOX is the human SMOX, such as provided in the following Accession Nos. NP_001257620.1, NP_787033.1, NP_787034.1, NP_787035.1 and NP_787036.1.

As used herein the term “disease associated with SMOX” refers to a disease associated with SMOX malfunction (e.g. due to a mutation) for onset and/or progression. A non-limiting example of such a disease is Syndromic X-Linked Intellectual Disability Snyder Type and Keratosis Follicularis Spinulosa Decalvans.

As used herein the term “ARRB1” [also known as Arrestin Beta, ARR1, Non-Visual Arrestin-2, Beta-Arrestin-1, Arrestin-2, Arrestin Beta-1 and ARB1] refers to the expression product e.g. RNA or protein of the ARRB1 gene (Gene ID 408). The gene encodes a cytosolic protein that acts as a cofactor in the beta-adrenergic receptor kinase (BARK) mediated desensitization of beta-adrenergic receptors, is expressed at high levels in peripheral blood leukocytes, and is believed to play a major role in regulating receptor-mediated immune functions.

According to specific embodiments, the ARRB1 is the human ARRB1, such as provided in the following Accession Nos. NM_004041.5, NP_004032.2, NP_064647.1 and NM_0202541.4.

As used herein the term “disease associated with ARRB1” refers to a disease associated with ARRB1 malfunction (e.g. due to a mutation) for onset and/or progression. A non-limiting example of such a disease Precocious Puberty, Central, 1 and Nephrogenic Syndrome Of Inappropriate Antidiuresis.

As used herein the term “ADCY6” [also known as Adenylate Cyclase 6, AC6, Ca(2+)-Inhibitable Adenylate Cyclase, ATP-Pyrophosphate Lyase 6, Adenylate Cyclase Type VI, Adenylate Cyclase Type 6, EC4.6.1.1, Adenylyl Cyclase 6] refers to the expression product e.g. RNA or protein of the ADCY6 gene (Gene ID 112). The gene encodes a protein that is a member of the adenylyl cyclase family of proteins, which are required for the synthesis of cyclic AMP. All members of this family have an intracellular N-terminus, a tandem repeat of six transmembrane domains separated by a cytoplasmic loop, and a C-terminal cytoplasmic domain. The two cytoplasmic regions bind ATP and form the catalytic core of the protein.

According to specific embodiments, the ADCY6 is the human ADCY6, such as provided in the following Accession Nos. NM_001390830.1, NP_001377759.1, NM_015270.5 and NP_056085.1.

As used herein the term “disease associated with ADCY6” refers to a disease associated with ADCY6 malfunction (e.g. due to a mutation) for onset and/or progression. A non-limiting example of such a disease is Lethal Congenital Contracture Syndrome 8 and Hypomyelination Neuropathy-Arthrogryposis Syndrome.

As used herein the term “FOXO3” [also known as Forkhead Box 03, FOXO3A, FOXO2, AF6q21, Forkhead Box Protein 03, FKHRL1P2 and AF6q21 Protein] refers to the expression product e.g. RNA or protein of the FOXO3 gene (Gene ID 2309). The gene encodes a transcriptional activator which belongs to the forkhead family of transcription factors which are characterized by a distinct forkhead domain and regulate processes such as apoptosis and autophagy.

According to specific embodiments, the FOXO3 is the human FOXO3, such as provided in the following Accession Nos. NM_201559.3, NP_963853.1, NM_001455.4 and NP_001446.1.

As used herein the term “disease associated with FOXO3” refers to a disease associated with FOXO3 malfunction (e.g. due to a mutation) for onset and/or progression. A non-limiting example of such a disease is Aging and Chromosome 6Q Deletion.

As used herein the term “STOM” [also known as Stomatin, BND7, EPB72, Erythrocyte Membrane Protein Band 7.2 and Erythrocyte Surface Protein Band 7.2] refers to the expression product e.g. RNA or protein of the STOM gene (Gene ID 2040). The gene encodes a member of a highly conserved family of integral membrane proteins. The encoded protein localizes to the cell membrane of red blood cells and other cell types, where it may regulate ion channels and transporters.

According to specific embodiments, the STOM is the human STOM, such as provided in the following Accession Nos. NM_004099.6, NP_004090.4, NM_198194.3 and NP_937837.1, NM_001270526.2 and NP_001257455.1.

As used herein the term “disease associated with STOM” refers to a disease associated with STOM malfunction (e.g. due to a mutation) for onset and/or progression. A non-limiting example of such a disease is Overhydrated Hereditary Stomatocytosis and Cryohydrocytosis.

As used herein the term “DNAJB4” [also known as DNAJ Heat Shock Protein, Hsp40 Protein Homolog Subfamily B, member 4, HSP40 Homolog and DNAJW] refers to the expression product e.g. RNA or protein of the DNAJB4 gene (Gene ID 11080). The gene encodes a molecular chaperone, tumor suppressor, and member of the heat shock protein-4.0 family. The encoded protein binds the cell adhesion protein E-cadherin and targets it to the plasma membrane. This protein also binds incorrectly folded E-cadherin and targets it for endoplasmic reticulum-associated degradation. This gene is a strong tumor suppressor for colorectal carcinoma.

According to specific embodiments, the DNAJB4 is the human DNAJB4, such as provided in the following Accession Nos. NM_004099.6, NP_004090.4, NM_198194.3 and NP_937837.1, NM_001270526.2 and NP_001257455.1.

As used herein the term “disease associated with DNAJB4” refers to a disease associated with DNAJB4 malfunction (e.g. due to a mutation) for onset and/or progression. A non-limiting example of such a disease is Oculopharyngeal Muscular Dystrophy.

As used herein the term “TMCC2” [also known as Transmembrane And Coiled-Coil Domain Protein, HUCEP11, Cerebral Protein, FLJ38497 and KIAA0481] refers to the expression product e.g. RNA or protein of the TMCC2 gene (Gene ID 9911). The gene encodes an endoplasmic reticulum-based protein involved in amyloid precursor protein metabolism.

According to specific embodiments, the TMCC2 is the human TMCC2, such as provided in the following Accession Nos. NM_014858.4, NP_055673.2, NM_001242925.2 and NP_001229854.1, NM_001375652.1 and NP_001362581.1.

As used herein the term “disease associated with TMCC2” refers to a disease associated with TMCC2 malfunction (e.g. due to a mutation) for onset and/or progression. A non-limiting example of such a disease is Noonan Syndrome 10 and Deficiency Anemia.

As used herein the term “CPXM1” [also known as Carboxypeptidase X, M14 Family Member 1, CPX1, CPXM, Probable Carboxypeptidase X1, Metallocarboxypeptidase CPX-1] refers to the expression product e.g. RNA or protein of the CPXM1 gene (Gene ID 56265). The gene likely encodes a member of the carboxypeptidase family of proteins, which may be involved in cell-cell interactions.

According to specific embodiments, the CPXM1 is the human CPXM1, such as provided in the following Accession Nos. NM_019609.5, NP_062555.1, NM_001184699.2 and NP_001171628.1.

As used herein the term “disease associated with CPXM1” refers to a disease associated with CPXM1 malfunction (e.g. due to a mutation) for onset and/or progression. A non-limiting example of such a disease is Mirror Movements 1.

Thus, in some embodiments of the invention, diagnosing or monitoring an ADNP-associated disorder comprises determining a level of a marker selected from the group consisting of SMOX, ARRB1, ADCY6, FOXO3, STOM, DNAJB4, TMCC2 and CPXM1 in a biological sample of the subject.

In particular embodiments, levels of the markers can be used for monitoring efficacy of treatment in a subject diagnosed with a disease selected from the group consisting of autism spectrum disorder, neurodegenerative disease, cognitive deficit, mental disorder and cytoskeletal disorder, or in a subject diagnosed with a disease selected from the group consisting of autism spectrum disorder and intellectual disability, or in a subject diagnosed with Alzheimer's disease, where a decrease in the level of SMOX, ARRB1, ADCY6, FOXO3, STOM, DNAJB4 and/or TMCC2 and/or an increase in said level of CPXM1 following the treatment indicates the treatment is efficacious.

In other particular embodiments, levels of the markers can be used for diagnosing a disease selected from the group consisting of autism spectrum disorder, neurodegenerative disease, cognitive deficit, mental disorder and cytoskeletal disorder, diagnosing a disease selected from the group consisting of autism spectrum disorder and intellectual disability, or diagnosing Alzheimer's disease in a biological sample of the subject.

It will be appreciated that subjects having indications of positive diagnosis of Alzheimer's Disease, autism spectrum disorder, neurodegenerative disease, cognitive deficit, mental disorder and cytoskeletal disorder, autism spectrum disorder and intellectual disability according to the methods of present invention, can be referred for further diagnostic tests for confirmation of the diagnosis, and/or selected for treatment of the diagnosed condition.

One exemplary approach for further diagnosis of Alzheimer's disease, and other neurodegenerative diseases is clinical assessment with a set of diagnostic criteria developed by the National Institute of Neurological and Communicative Disorders and Stroke and the Alzheimer's Disease and Related Disorders Association (NINCDS-ADRDA) (McKhann et al, 19841. These criteria comprise aspects of medical history, clinical examination, neuropsychological testing, brain imaging, and laboratory assessments, and have recently been updated. Further laboratory assessment usually includes blood analysis (i.e., to exclude cognitive impairment due to nutrition deficiency or hormone disorders). Neuropsychological testing typically contains a test battery, or short screening instruments, e.g., the Mini-Mental Status Examination. Brain imaging is usually done via structural magnetic resonance imaging (MRI) or computed tomography, with positron emission tomography (PET) being added in cases with diagnostic uncertainty. Still further, radiological imaging may provide detection of pathological hallmarks of AD in vivo. While markers for Tau may still be experimental, a number of tracers binding to amyloid plaques are entering routine clinical use: ¹¹C-labeled Pittsburgh compound B (PIB) and ¹⁸F-labeled substitutes. After injection into the blood stream, Pittsburgh compound B (PIB) traverses the blood-brain barrier and binds to deposits of amyloid plaques (fibrillar amyloid-β peptides). PIB binding to amyloid plaques can be detected by PET. MRI scans are also useful, with development of automated classification in functional imaging and cortical thickness measurements to distinguish scans from patients with dementia. Multivariate pattern recognition methods (i.e., machine learning techniques) like support vector machines (SVMs) can also accurately diagnose dementia and AD.

In some embodiments, the method comprises treating the condition. Exemplary therapies for Alzheimer's disease, and other neurodegenerative conditions can include treatment with the ADNP polypeptide or peptide of the invention, as described herein, alone, or in combination with other drugs. A non-limiting list of drugs for Alzheimer's disease and other neurodegenerative disease includes Aducanumab, Donepezil, Revastigmine, Memantine, Memantine formulated with Donepezil and Galantamine. In addition, cognitive training can benefit.

In some embodiments, a threshold of each of SMOX, ARRB1, ADCY6, FOXO3, STOM, DNAJB4, TMCC2 and/or CPXM1 concentration or level is determined, and increased SMOX, ARRB1, ADCY6, FOXO3, STOM, DNAJB4 and/or TMCC2 above the predetermined threshold, and/or CPXM1 concentration or level below its predetermined threshold indicates the presence of a disease selected from the group consisting of autism spectrum disorder, neurodegenerative disease, cognitive deficit, mental disorder and cytoskeletal disorder, a disease selected from the group consisting of autism spectrum disorder and intellectual disability, or Alzheimer's disease in the subject. The predetermined threshold can be a value or range of values outside the range of SMOX, ARRB1, ADCY6, FOXO3, STOM, DNAJB4, TMCC2 and/or CPXM1 concentrations determined for normal, healthy human subjects, or may be a value of SMOX, ARRB1, ADCY6, FOXO3, STOM, DNAJB4, TMCC2 and/or CPXM1 concentration assayed in a sample or samples taken from matched subject or subjects (e.g. matched for at least one of the following criteria: age, BMI, weight, prior diagnosis and/or family history of autism spectrum disorder, neurodegenerative disease, cognitive deficit, mental disorder and cytoskeletal disorder, a disease selected from the group consisting of autism spectrum disorder and intellectual disability, or Alzheimer's disease, or other clinical parameters) who do not have autism spectrum disorder, neurodegenerative disease, cognitive deficit, mental disorder and cytoskeletal disorder, a disease selected from the group consisting of autism spectrum disorder and intellectual disability, or Alzheimer's disease. The reference sample for determining the threshold can also be a prior sample taken from the same subject (as in monitoring). In some embodiments, “increased” refers to concentrations significantly, in the range of 5%, 10%, 15%, 20%, 25%, 30%, 35%, 40%, 45%, 50%, 55%, 60%, 65%, 70%, 75%, 80%, 85%, 90%, 95%, 100%, 1.5-fold, 2 fold, 3 fold, 4 fold or more greater than the predetermined threshold. In other embodiments, “below” or “decreased” refers to concentrations significantly, in the range of 5%, 10%, 15%, 20%, 25%, 30%, 35%, 40%, 45%, 50%, 55%, 60%, 65%, 70%, 75%, 80%, 85%, 90%, 95%, 100%, 1.5-fold, 2 fold, 3 fold, 4 fold or more lower than the predetermined threshold.

In some aspects of the invention, the method comprises detecting markers selected from the group consisting of SMOX, ARRB1, ADCY6, FOXO3, STOM, DNAJB4, TMCC2 and CPXM1 in a biological sample of the subject. The term “biological sample” encompasses a variety of sample types obtained from an organism and can be used in a diagnostic, prognostic, or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples. Clinical samples for use in the methods of the invention may be obtained from a variety of sources, particularly blood samples.

Sample sources of particular interest include blood samples or preparations thereof, e.g., whole blood, or serum or plasma, tears and urine. In specific embodiments, a suitable initial source for the human sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood, allowed to clot, and the serum or plasma separated and collected to be used to assay. In other embodiments, the sample is derived from blood collected without clotting (e.g. along with anti-coagulant such as EDTA, citrate, heparin) and then serum or plasma collected for assay.

In some embodiments the sample is a serum or serum-derived sample. Any convenient methodology for producing a fluid serum sample may be employed.

Also provided are reagents, systems and kits thereof for practicing one or more of the above-described methods. The subject reagents, systems and kits thereof may vary greatly. Reagents of interest include reagents specifically designed for use in producing clinically useful marker level representations of the above-described markers from a sample, for example, one or more detection elements, e.g. antibodies or peptides for the detection of the marker protein, oligonucleotides for the detection of nucleic acids, etc. In some instances, the kit comprises a first agent which specifically binds soluble SMOX, ARRB1, ADCY6, FOXO3, STOM, DNAJB4, TMCC2 or CPXM1, and normal reference samples for the markers. In specific embodiments, the normal reference sample for SMOX, ARRB1, ADCY6, FOXO3, STOM, DNAJB4, TMCC2 and CPXM1 is a sample containing a level of the marker(s) found in a healthy human subject not having any of the ADNF/ADNP-associated conditions detailed hereinabove (e.g. autism spectrum disorder, neurodegenerative disease, cognitive deficit, mental disorder and cytoskeletal disorder). In other embodiments, the agents may be detectably labeled or coupled to an enzyme, e.g. a fluorescent labeled, radioactive labeled or immunoconjugated antibody. The agents binding the markers can be an antibody to the marker, or marker-binding fragment thereof. In some embodiments, the antibody is a monoclonal antibody. In other embodiments, the agent binding the marker(s) is immobilized on a solid surface, e.g. bound to beads (microspheres), ELISA plate, etc, as detailed hereinabove.

In some embodiments, the system or kit comprises a test strip (e.g. lateral flow test strip) also known as dipstick, preferably, though not necessarily, encased in a housing, designed to be read by the subject or medical professional, and in some embodiments, the assay performed with the test strip is a sandwich immunoassay. Usually additional molecules are present in a device as a positive or negative control. A typical positive control could be an antibody recognizing a molecule which is known to be present in a sample to be tested. A typical negative control could be an antibody recognizing a molecule which is known to be absent in a sample to be tested.

Another type of such agent is an array of probes, collections of primers, or collections of antibodies that include probes, primers or antibodies (also called reagents) that are specific for SMOX, ARRB1, ADCY6, FOXO3, STOM, DNAJB4, TMCC2 or CPXM1. Such an array may include reagents specific for additional genes/proteins/cofactors that are not listed above, such as probes, primers, or antibodies specific for genes/proteins/cofactors whose expression pattern are known in the art to be associated with the diseases and conditions detailed herein.

In some instances, a system may be provided. As used herein, the term “system” refers to a collection of reagents, however compiled, e.g., by purchasing the collection of reagents from the same or different sources. In some instances, a kit may be provided. As used herein, the term “kit” refers to a collection of reagents provided, e.g., sold, together. For example, the antibody-based detection of the sample proteins, respectively, may be coupled with an electrochemical biosensor platform that will allow multiplex determination of SMOX, ARRB1, ADCY6, FOXO3, STOM, DNAJB4, TMCC2 or CPXM1 for personalized care. The systems and kits of the subject invention may include the above-described arrays, gene-specific primer collections, or protein-specific antibody collections, as well as one or more additional reagents employed in the various methods, which may be either premixed or separate. The subject systems and kits may also include one or more preeclampsia phenotype determination elements, e.g. a reference or control sample or marker representation that can be employed, e.g., by a suitable experimental or computing means, to make a preeclampsia prognosis based on an “input” marker level profile.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The polypeptides and/or therapeutic agents described herein can be provided to the subject per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide or therapeutic agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, sublingual, topical, intra-dermal, rectal, transmucosal (including eye drops), especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, intrapulmonary or intraocular injections.

According to specific embodiments, the active ingredients are provided in a systemic manner.

According to specific embodiments, the route of administration is intranasal or intrapulmonary administration.

According to specific embodiments, the polypeptide is formulated for nasal administration as described in WO16/073,199, the contents of which are fully incorporated herein by reference.

According to other specific embodiments, the route of administration is into the skin. Methods of administering an active agent into a skin are known in the art and include, for example, intradermal injections, gels, liquid sprays, devices and patches which comprise the active agent and which are applied on the outer surface of the skin.

According to some embodiments of the invention, administration of the active agent into the skin of the subject is performed topically (on the skin).

According to some embodiments of the invention, administration of the active agent into the skin of the subject is performed non-invasively, e.g., using a gel, a liquid spray or a patch (e.g. reservoir type patch and matrix type patch) comprising the active ingredient, which are applied onto the skin of the subject.

It should be noted that in order to increase delivery of the active agent into the skin, the active agent can be formulated with various vehicles designed to increase delivery to the epidermis or the dermis layers. Such vehicles include, but are not limited to liposomes, dendrimers, noisome, transfersome, microemulsion and solid lipid nanoparticles.

According to some embodiments of the invention, administering is performed by an intradermal injection.

Conventional approaches for drug delivery to the central nervous system (CNS) include: neurosurgical strategies (e.g., intrahippocampal (IH), intracranial (IC), intracerebral injection, intracerebroventricular injection (ICV) or infusion or intrathecal administration); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer, or saline or slow-release solutions. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

According to specific embodiments, the pharmaceutical composition is formulated for oral administration.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

According to specific embodiments, the pharmaceutical composition is formulated for inhalation (e.g. intranasal or intrapulmonary).

For administration by inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents. Slow release formulations may also be used in preparation of the pharmaceutical composition for parenteral administration.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

The pharmaceutical composition of some embodiments of the invention may also be formulated for sustained-release to provide elevated serum half-life. Such sustained release systems are well known to those of skill in the art and include e.g. microcapsules and nanoparticles. According to specific embodiments, the ProLease biodegradable microsphere delivery system for proteins and peptides (Tracy, 1998, Biotechnol. Prog. 14, 108; Johnson et al., 1996, Nature Med. 2, 795; Herbert et al., 1998, Pharmaceut. Res. 15, 357) a dry powder composed of biodegradable polymeric microspheres containing the protein in a polymer matrix that can be compounded as a dry formulation with or without other agents.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., ARDS, infectious diseases e.g. Corona virus infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

For complete toxicity assessment of the ADNP polypeptide of SEQ ID NO: 2 see Gozes I. Front Neurol. 2020 Nov. 24; 11: 608444, the contents of which are fully incorporated herein by reference.

Dosage amount and interval may be adjusted individually to provide that the levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

The doses determined in a mouse model can be converted for the treatment other species such as human and other animals diagnosed with the disease. Conversion Table approved by the FDA is shown in Reagan-Shaw S., et al., FASEB J. 22:659-661 (2007).

The human equivalent dose is calculated as follows: HED (mg/kg)=Animal dose (mg/kg) multiplied by (Animal Kₘ/human Kₘ).

Depending on the severity and responsiveness of the condition to be treated, dosing can be of a single or a plurality of administrations, with course of treatment lasting from several days to several weeks or until cure is effected or diminution of the disease state is achieved. It will be appreciated that treatment will typically be an extended, and in most cases chronic course of treatment, as the target patient population comprises genetically impaired individuals, whose neurodegenerative conditions require ongoing attention.

According to specific embodiments, the polypeptide is administered once or twice a day.

The amount of a composition to be administered will, of course, be dependent on the subject being treated, the severity of the affliction, the manner of administration, the judgment of the prescribing physician, etc.

According to some embodiments of the invention, the polypeptide is provided at an amount ranging from 0.0001 mg/kg to 1,000 mg/kg including any intermediate subranges and values therebetween, e.g. 0.001 mg/kg, 0.1 mg/kg, 1 mg/kg, 5 mg/kg, 15 mg/kg, 50 mg/kg or 500 mg/kg per dose. According to specific embodiments, the polypeptide is provided in an amount ranging from 0.05-0.1 mg/kg e.g. 0.08 mg/kg. In more specific embodiments, the polypeptide is provided in an amount ranging from 0.01 mg/kg to 2 mg/kg body weight, including any intermediate subranges and values therebetween, e.g. 1, 2, 3, 4, 5, 6, 7, 8, 9, 10, 11, 12, 13, 14, 15, 16, 17, 18, 129, 20, 21, 22, 23, 24, 25, 26, 27, 28, 29, 30, 31, 32, 33, 34, 35, 36, 37, 38, 39, 40, 41, 42, 43, 44, 44, 45, 46, 47, 48, 49 or 50 mg/70 kg subject. In yet further embodiments, the polypeptide is provided in an amount ranging from 1 to 40 mg/70 kg subject, in particular 5, 15 or 30 mg/70 kg subject.

According to specific embodiments, the polypeptide is provided in an amount ranging from 0.1-1 mg/kg e.g. 0.4 mg/kg given e.g. subcutaneously.

According to specific embodiments, the polypeptide is provided in an amount ranging from 0.05-0.5 mg/kg e.g. 0.2 mg/kg (15 mg to a 70 kg subject) or 0.07 mg/kg (5 mg to a 70 kg subject) e.g. intranasally.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Novel Effects of Nap Discovered in an Adnp Syndrome Mouse Model

### Materials and Methods

Generation, Genotyping and Breeding of Tyr Mice—Mice were Generated Using Standard Methods, Described in e.g. (99).

Specifically, 5 different G2 mice were bred to G4 to dilute possible off target affects. Breeding scheme for all experiments was as follows: Tyr male X C57BL6/NJ females. C57BL6/NJ colony was kept under 5 breeding generations at our facility and refreshed using mice received from Jackson laboratories to avoid genetic drift and shift. Mice were genotyped at either p0-1 or at weaning depending on the experimental group using tail snippings and were either tagged or tattooed (Ketchum, Canada). To determine sex at P0-1, mice were also genotype for the presence of a Y chromosome using primers for SRY.

Animals—Animals were housed under a 12-hour light/dark cycle, with food and water ad-libitum. Mouse pups were subcutaneously administrated with NAP [having an amino acid sequence NAPVSIPQ (SEQ ID NO: 2), 25 μg NAP/1 ml saline] as described (8, 12). At 21 days of age mice were administered daily intranasal NAP in vehicle solution (“DD”—7.5 mg/ml of NaCl, 1.7 mg/ml of citric acid, 3 mg/ml of disodium phosphate dihydrate, and 0.2 mg/ml of 50% benzalkonium chloride solution) (0.5 μg NAP/5p1 DD) (8, 9, 12, and U.S. Pat. No. 10,912,819 to Gozes). These mice continued to behavioral analysis as described hereinbelow. The noninvasive intranasal route was chosen as these experiments required daily handling and administration. Age (weeks) of mice during behavioral experiments (mean±SEM) was as follows: Catwalk-9.23±0.15, grooming-10±0.15, social approach-10.5±0.12, odor-discrimination-12.12±0.13, hanging-wire-13±0.14, hotplate-13.18±0.13. Group size was determined following previous experiments (12). Pups were randomly allocated to either the NAP or DD group according to sex and genotype. All procedures involving animals were conducted under the supervision and approval of the institutional animal care and use committee of Tel Aviv University and the Israeli Ministry of Health.

Developmental milestone assessment and fostering—This experiment included animals from 16 litters in 2 experimental batches. It was performed as previously described (12) with slight modifications. Experimenters were blinded to sex and genotype during experimental procedures. Sexes were grouped if no statistically significant sex differences were discovered.

Catwalk—Was performed as previously described (12).

Additional behavioral assessments—

Fostering: To enable daily handling necessary for developmental milestone assessment and drug treatment, Tyr pups were fostered by ICR females which are known to be excellent dams. Tyr pups were moved after birth to an ICR female mouse who gave birth up to 48 hours prior. ICR litter was culled according to the number of Tyr pups that were added. One day afterwards the remaining ICR pups were euthanized so only Tyr pups were left in the litter.

Pup litter distribution analysis: The genotyping results at p1 after placement with the foster mother was used for this measurement. Hence the decrease in observed Tyr pups either represents death in utero or death between p0 to p1.

Developmental milestone assessment: At p1 Tyr pups were tattooed and genotyped for the presence of the Tyr718 mutation and SRY as described above. The same day pups were randomized to either NAP or Saline treatment groups according to genotype and sex results and developmental milestone assessment was performed from p1 to p21. As animal randomization ended up with unequal male/female ratios for certain groups and no statistical differences were found between the sexes in any of the 4 treatment groups (T test), the sexes could not be separated, [That is except for eye opening, where a significant difference was observed between the sexes in the Tyr group (P=0.047, no significance in the other three treatment groups)]. Pup paws were tattooed using a 31-gauge needle and tattoo ink (Ketchum Manufacturing Co., catalog 329AA) for identification on P1 and, until P21, underwent daily measurements for length and weight and observation for eye opening. Pups were injected daily with NAP, 1 hour prior to the tests as previously described¹.

Briefly, for ear twitch reflex, a cotton tip was gently brushed against the tip of the ear three times, between postnatal days 7-15 or until the pup responded correctly for two consecutive days. For air righting reflex, the pup was held up-side down and released ˜10.5 cm above the cage, containing 5 cm of shavings. The test was performed between postnatal days 8-21 or until the pup landed on the shavings with all four paws, for two consecutive days. For surface righting reflex, pups were placed on their backs on a level surface, the test was performed between postnatal days 1-13 until the pups was able to right itself with all four paws on the surface in under a second for two consecutive days. For eye opening, mice were observed daily until both eyes were open (eyelids separated completely). For auditory startle, the pup was placed on a level surface and the experimenter clapped loudly twice until a visible startle response was evident for 2 consecutive days. The test was performed between days 7-18. For cliff aversion, pups were placed on the edge of a small box (approx. 4 cm in height) with both paws protruding over the edge of the box. Pups were allowed 30 sec to avoid the cliff and move themselves backwards. Test was performed between day 1-14 until the pup avoided the cliff in under 30 sec for two consecutive days⁹. For the rooting reflex, a cotton tip was gently brushed against the sides of the pup's mouth until the pup moved its head to the direction of the stroke. Both sides were tested twice. Test was performed between days 1-12 until the pup had a visible response to the cotton tip stroke. All tests were conducted between 10 AM-4 PM. For the first 5 postnatal days, each pup was put under a single 60-watt bulb during testing to assure the pups' warmth.

Novel object recognition: Briefly, the open field test was used for the first day of habituation since both tests are conducted in the same arena. The following day animals underwent another habituation to the arena (5 mins). As described, the test included two consecutive days of habituation and the experimental days which consisted of the three phases. In phase 1 (habituation), the open field apparatus (50×50 cm) contained two identical objects (plastic or metal, 4×5 cm2) and a mouse was placed in the apparatus facing the wall and allowed to freely explore the objects (5 min). After 3 h in the home cage, the mouse was placed back into the apparatus for 3 min for phase 2 (short retention choice phase—data not shown), during which one of the familiar objects was replaced with a novel object. Approximately 24 h after the completion of phase 2, the mouse was placed into the apparatus for the long retention choice phase, during which one of the familiar objects was replaced with a novel object. The mouse was kept in its home cage between phases 2 and 3. The time spent sniffing/touching each object was measured using the EthoVision XT video tracking system and software (Noldus Inc. Leesburg, VA). Data were analyzed using the discrimination capacity formula: D2=(b−a)/(a+b), where ‘a’ designated the time of exploration of the familiar object and ‘b’ designated the time of exploration of the novel object.

Social approach: A plexiglas box was divided into three adjacent chambers, each 20 cm (length)×40.5 cm (width)×22 cm (height), separated by two removable doors. Steel wire cups (10.16 cm (diameter), 10.8 cm (height)), were used as both containment for the target mice and as inanimate objects Experiments were conducted in a dimly lit area during the light phase of the mouse. Target mice (males for males and females for females) were placed inside the wire cup in one of the side chambers for three 10-min sessions on the day before the test for habituation. The next day, each subject mouse was tested in an experiment with three phases, (measured with a simple timer): I and II (5 minutes long), the habituation phases (ensuring no bias), and III (10 minutes long), the experimental phase. In phase III, an empty wire cup (novel object) was placed in the center of the right or left chamber and the cup containing the target mouse was placed in the center of the other chamber. Location of the empty wire cup (novel object) and the novel mice were counterbalanced to avoid confounding side preference. The doors were then removed and a 10-min timer was initiated. The three-chamber apparatus was cleaned between mice. Mouse movement and exploratory behavior were tracked and recorded using the EthoVision XT video tracking system and software (Noldus Inc. Leesburg, VA).

Odor discrimination: In short: odors were presented on a piece of cotton, hidden inside a perforated small object placed in the corner of the arena (20×40). The test mouse was placed into a clean cage with fresh shavings. Each mouse was tested during three consecutive 2-min periods for each odor, with 2-min intervals between presentations and 3-min intervals between presentation of different odors. The x axis indicates the consecutive number of the odor exposure period. The time that the mouse smelled the swab was recorded using the EthoVision XT video tracking system and software (Noldus Inc. Leesburg, VA).

Grooming: Test mice were placed inside a 50×50 white Plexiglas arena for 15 minutes. Mouse movement and exploratory behavior were tracked and recorded using the EthoVision XT video tracking system and software (Noldus Inc. Leesburg, VA), which also allows for grooming behavior detection.

Hanging wire test: Three trials were performed for each mouse. An assessment of the mouse paws' strength was performed by measuring their latency to fall off a metal wire (placed 50 cm above the surface) onto soft bedding (maximum time of 90 s).

Hotplate: mice were placed on a hotplate at 55° c. Latency to nociception was determined until the mouse jumped or licked its hindpaws. Maximum time was 90 s.

CatWalk: Briefly, The CatWalk XT (Noldus Inc. Leesburg, VA) was used to analyze the gait of unforced, moving mice¹⁰ Mice had to cross the runway of the CatWalk XT apparatus in a consistent manner, and a successful run was defined when an animal ran the track without any interruption or hesitation. Every mouse was tested until 3 successful runs were achieved.

Base of support is the average width between either the front paws or the hind paws. Swing (sec) or swing phase is the duration in seconds of no contact of a paw with the glass plate. Swing Speed is the speed (Distance Unit/second) of the paw during Swing. Max Contact Max Intensity is the maximum Intensity at Max Contact of a paw. Intensity ranges from 0 to 255 in arbitrary units. The intensity of a print depends on the degree of contact between a paw and the glass plate and increases with increasing weight. Therefore, Intensity is a measure of weight put on the glass plate.

The Intensity parameter is used to assess the effects of neuropathic pain, including mechanical allodynia. Max Contact Mean Intensity is the mean Intensity of a paw at Max Contact. Print length is the length (horizontal direction) of the complete print. The complete print is the sum of all contacts with the glass plate, as if the animal's paw would have been inked. Print width is the width (vertical direction) of the complete paw. Print area is the surface area of the complete print. Max Intensity is the maximum Intensity of the complete paw. Mean intensity is the mean Intensity of the complete paw.

USV: On P8, each pup was separated from the dam and placed in an empty cage for 6 minutes, in a quiet room and was recorded using the M500-384 USB Ultrasound Microphone (Petterson, Sweden). Pups were distinguished from one another using the tattoos listed above. After 6 minutes pups were reunited with its dam. Data were analyzed using DeepSqueak. Data were loaded onto DeepSqueak, frequencies between 40-70 kHz were analyzed using the mouse call network V2. Calls were denoised using post hoc denoising network. For syntax analysis, Syntax was classified using Wright K means network. Syntax transition probability charts were generated using DeepSqueak and Matlab. Further USV experiments were performed on P8 pups as previously described (12) with slight modifications.

Spine quantitation—For determination of dendritic spine morphology, the Tyr-GFP mouse model was used and generated as described(12). 1.5-2-month-old Tyr-GFP-mice were treated for 9 consecutive days with either intraperitoneal NAP injection (0.4 μg/0.1 ml saline) or with 0.1 ml saline. On day 9, mice were perfused, and brains were subjected to immunohistochemistry and imaging (12).

AT8 Immunohistochemistry—experiment was performed as previously described (100). Mice were treated for 6 weeks with daily intranasal DD/NAP and were euthanized at 10.21±0.05 weeks of age.

VEP—5.72±0.52-week-old male mice were used for VEP measurements. Animals were treated for 3 days with 0.1 ml S.C saline, recorded, and then followed by 4 daily S.C doses of either 1 μg NAP (in 0.1 ml saline) or 0.1 ml saline followed by another VEP recording.

RNA seq—Total RNA was extracted from 14-week-old mouse spleens and sequenced using standard methods on NextSeq500 system (Illumina) as detailed. Data were analyzed as previously described (9).

Microbiome—Fecal samples were collected from 12.2±0.19-week-old mice. Samples were prepared and analyzed as previously described (57). Microbiome data was analyzed after applying the logarithmic transformation.

Statistical Analysis—All statistical methods are described in the appropriate figure legends. P<0.05 was considered statistically significant, and all tests were 2 tailed. Data were checked for normal distribution by normality test. Results are presented as the mean±SEM. For 2 different categorically independent variables, a two-way ANOVA or two way, repeated-measures ANOVA followed by Tukey's post hoc was performed. An unpaired Student's t test, Mann-Whitney U test or one-way ANOVA, Xi square or binomial tests were performed when applicable. Pearson correlation was performed for microbiome data correlation. For developmental milestone measurements, in vivo behavioral tests, microbiome data and dendritic spine quantification, outlier values were excluded using the Grubbs test. Statistical analyses were performed on SigmaPlot 12.5 (Systat Software Inc., CA, USA) or Prism 8 (GraphPad, CA, USA) software.

### Results

### Genome Editing and Mouse Model Generation:

A novel mouse model (hereinafter “Tyr”) carrying the NM_001310086 (Adnp_v001):c.2154T>A, p.Tyr718* mutation (homologous to the most common human ADNP p.Tyr719* mutation) was generated using the standard CRISPR-Cas9 technology and characterized (below).

The Tyr genotype has a major effect on early life events with NAP correction: ADNP syndrome patients suffer from developmental delays(24). Hence, the present inventors explored whether Tyr mice displayed similar anomalies and if those were reversible by treatment with the ADNP polypeptide NAP [having an amino acid sequence NAPVSIPQ (SEQ ID NO: 2)]. As the background strain C57bl/6NJ dams did not enable daily pup handling, NAP administration and testing, foster ICR dams were used. Immediate post-fostering genotyping detected a significant reduction in Tyr P1 live pups from the expected 50% to 43% (FIG. 1A). Surviving Tyr mice showed delayed acquisition of vestibular reflexes (FIG. 1B). Both Surface-righting (the pup's ability to right itself on all 4 paws on a flat surface) and air-righting (the ability to right to the correct orientation mid-air, measured as the first day of acquisition) were delayed due to genotype (WT: 8.69±0.42 and Tyr: 10.14±0.36, WT: 12.96±0.28 and Tyr: 15.29±0.15, respectively). NAP showed a significant curative effect on both reflexes, almost normalizing surface-righting (8.81±0.33) and promoting the air-righting response (14.63±0.38). Furthermore, genotype differences were discovered in the ear-twitch and cliff aversion behaviors (data not shown). Intriguingly, Tyr pups began hearing significantly later vs. controls, as demonstrated in the auditory startle reflex (data not shown). Additionally, NAP induced rooting in Tyr mice and had no effect on WT littermates except for the cliff aversion response (data not shown).

Furthermore, with brain development known to influence facial development (38), it was found that female Tyr mice open their eyes a day later than WT females (16±0.37 and 14.93±0.43, respectively), with NAP completely reversing this effect (14.78±0.46) (FIG. 1C). Here, sexes were separated due to statistical differences. No genotype/NAP effect was observed on males (data not shown). Similarly, no NAP effect was observed in WT females (data not shown).

Additionally, ADNP syndrome patients present impaired weight and short stature (24, 25). Hence, weight and length in Tyr pups were evaluated. Female Tyr pups had lower body weight than WT littermates, starting from P10 (FIG. 1D). NAP beneficial effect was visible from P10 and reached significance at P20-21, with no effect observed on WT female mice (data not shown). Tyr female pups also exhibited a shorter length, compared to WT pups starting from P10 (data not shown). No significant differences were observed for male pups' weight and length, and no major differences were observed for adult weights (data not shown).

Tyr mice show abnormal behavioral, motor, social and sensory traits: Naïve male Tyr mice (not exposed to foster dams or any previous handling) showed long-term memory impairment in the novel object recognition test (data not shown). Furthermore, both sexes of naïve Tyr mice showed impairments in the social approach task (data not shown). These differences were lost in the treated cohort (data not shown), possibly due to early handling (39) and fostering (39-41), suggesting that early behavioral intervention treatment might be useful for ADNP children (25). Regardless, repetitive behavior, a key ASD feature, was evaluated by measuring grooming frequency and duration (42). Tyr mice showed increases in both parameters (data not shown). A genotype effect in the hot-plate (sensory) and hanging-wire (motor) tests was also observed (data not shown). In addition, both male and female Tyr mice showed impaired odor discrimination compared to WT, with NAP reversal in females (data not shown). NAP showed varied minor effects on WT mice (data not shown).

### NAP Protects Against Gait Impairments Caused by the Tyr Mutation:

ADNP syndrome patients exhibit delayed and abnormal gait (24, 25). In this respect, when looking at the Tyr mice, genotype abnormalities were observed in multiple gait parameters, measured by the Catwalk-XT system (data not shown). Interestingly, a marked sex difference was observed. FIG. 1E groups male results, with Tyr mice exhibiting increases in print length and area vs. WT mice, with significant NAP recovery. Both male and female Tyr mice showed a smaller front base of support, with NAP correcting this phenotype in males. In FIG. 1F female Tyr mice showed slower swing speeds, accompanied by increased swing durations, compared with WT females, ameliorated by NAP. A significant decrease in various intensity parameters when comparing Tyr and WT females with significant NAP correction was also discovered. Altogether, these results showed that gait was significantly impaired by the Tyr genotype, with a more severe phenotype in females, improved by NAP treatment. No major NAP effect was observed in WT mice (data not shown).

Ultrasonic Vocalizations (USVs) are Dramatically Impaired in Tyr Mice with NAP Correction:

ASD and ADNP syndrome affect early development, with many patients suffering from severe impact on speech and communication delays (24, 25, 43). Therefore, Tyr vocalization was evaluated using pup-dam separation (a reliable and reproducible way to produce USVs (44)). Since sex differences have been previously described in P8 mice pup USVs (45), sexes were analyzed separately. A significant genotype decrease in total USVs produced by females (from 186.6±24.36 to 64.78±17.16) and males (from 149.8±31.07 to 40.36±9.88) was observed, with no NAP effects.

Syntax of syllables elicited by pups has been demonstrated to be non-random and change with development, thus representing a facet of ASD/ID (46). Mechanistically, the change in complexity may be a way of the pup to distinguish itself from the rest of the litter, increasing nourishment chances (46). DeepSqueak (47) was used to evaluate syntax complexity. An evident decrease in complexity was discovered between WT and Tyr females (FIG. 2A), with Tyr females losing the Trill syllable and performing less transitions. In males (FIG. 2B), similar marked decreases in complexity were observed. Importantly, NAP showed a powerful corrective effect on both sexes. NAP-treated Tyr males vocalized twice more syllables than WT and Tyr pups (8 for WT and Tyr and 10 for Tyr NAP with addition of Split and Trill syllables). Statistic quantification of this effect (FIGS. 2C-D) demonstrated that Tyr pups performed worse than WT littermates and NAP ameliorated the effect. Also, NAP had a minor varied impact on WT pups (data not shown).). WT females exhibited richer syntax than WT or Tyr males (data not shown), reflecting sexual differences.

Dendritic Spine Density and Morphology are Significantly Impaired in Tyr Mice, Coupled with NAP Correction:

Dendritic spine abnormalities have long been considered to be an important molecular cause of ASD (48), coupled with ADNP's known functions at this site (12, 49). Here, significant genotype alterations with striking sexual dichotomy were discovered. Male Tyr mice showed a ˜15% reduction in CA1 hippocampal dendritic spines (FIG. 3A), with complete NAP reversal (˜23% increase in spine density, FIG. 3B). When dividing spines into different morphological subtypes (mushroom, stubby and thin), a similar pattern was observed in males, with no genotype or treatment changes in females or treatment effect on WT males with regards to total spines and subtypes (data not shown). Mirroring male hippocampal effects, a significant ˜16% reduction in spine density was observed in the female motor cortex L5 layer (FIG. 3A), coupled with complete NAP amelioration (˜17% increase, FIG. 3B). No genotype or treatment changes were observed in males and no treatment effect was found on WT females (data not shown). When dividing the spines into different subtypes, the same genotype effects were observed in the mushroom and stubby spines, with NAP significantly affecting the larger, functionally stronger, mushroom shaped spines (FIG. 3A). No NAP effect was found in males and WT females (data not shown). Looking closely at PSD95 volume in the hippocampus, a ˜25% genotype decrease was observed in females only, with complete resolution upon NAP treatment (FIG. 3A).

### Male Tyr Mice Show Hyperphosphorylated Tau Deposits in the Hippocampus and Visual Cortex:

Next, the present inventors sought out to further investigate cellular insults instigated by the Tyr mutation. Following recent findings of tauopathy in a deceased ADNP syndrome patient (27), it was wondered whether young Tyr mice showed evidence of tauopathy. The present inventors discovered that Tyr males had a significant ˜1.84-fold increase of AT8 (pathological Tau hyperphosphorylation) positive cells, with complete NAP reversal (˜2.57-fold decrease) in the hippocampus (FIGS. 3C-D). This finding was further extended to the visual cortex, demonstrating the same genotype effect on males: a significant ˜1.7-fold increase in AT8 staining, coupled with a NAP significant reversal by ˜2-fold (FIGS. 4A-B). Females did not exhibit significant effect and WT males did not display a NAP effect (data not shown). Another hyperphosphorylated tau-specific antibody (AT180) exhibited similar trends, and combining AT8- and AT180-positive cells revealed a significant Tyr genotype increase with correction by NAP in both sexes (data not shown).

Male Tyr Mice Show Abnormal Visual Evoked Potentials (VEP) with NAP Amelioration:

Having obtained the above tauopathy results, the present inventors asked whether these degenerative changes impacted brain synaptic functionality. In this respect, VEPs have been linked with AD pathology (50) and most importantly with ASD (51). Furthermore, severe visual impairments have been previously reported in ADNP syndrome patients (52, 53). Early preliminary experiments showed a more robust and reproducible phenotype in Tyr males vs. females (data not shown); hence, NAP effect was evaluated in Tyr males. Striking differences were observed between Tyr and WT males, with short-term NAP treatment (4 consecutive daily doses) normalizing these effects (FIG. 4C). The area under the curve (AUC) of three VEP major waveforms (P2, N2 and P3) as a response to three different stimulation intensities (100, 300 and 3000 mcd, FIG. 4D) showed a significant genotype effect at P2 (˜1.7-fold increase in all intensities), with an almost complete NAP reversal (˜1.6-2.3-fold decrease). The same significant change was observed for N2 (FIG. 4E). As for P3 (FIG. 4F), a significant genotype effect was detected in all 3 intensities, with a significant NAP effect seen in the 300 and 3000 mcd intensities.

Female Tyr mice differentially expressed genes reveal specific blood biomarkers: ADNP syndrome children suffer from recurrent infections, compared to their healthy siblings (24), attesting to a potentially impaired immune system. Furthermore, previous RNA-seq results revealed major shared immune-related changes in lymphoblastoid cells harboring several ADNP syndrome mutations (12, 27). Lastly, blood DNA methylation patterns, epi-signatures stratifying the ADNP syndrome show modest correlation with phenotype (54). Thus, RNA-seq was performed on spleens derived from Tyr mice, WT littermates and NAP-treated Tyr mice. The results (FIG. 5) revealed a very minor genotype/NAP effect on males, and a more comprehensive effect on females, with 5 common transcripts, namely SMOX (Spermine Oxidase), ARRB1 (Arrestin Beta 1), ADCY6 (Adenylate Cyclase 6), FOXO3 (Forkhead Box 03), and CPXM1 (Carboxypeptidase X, M14 Family Member 1), shared as deregulated by all the previously tested patient-derived ADNP-mutated lymphoblastoid cells. Two additional ADNP/NAP-regulated proteins were shared only with the human homologous mutation p.Tyr719* [STOM (Stomatin), DNAJB4 (DnaJ Heat Shock Protein Family (Hsp40) Member B4], as well as TMCC2 (Transmembrane And Coiled-Coil Domain Family 2) (with the p.Lys408Valfs*31 mutation), previously found to regulate amyloid precursor protein (55). A database of secreted proteins revealed that most of these proteins can be found in the human plasma, suggesting novel biomarkers for the ADNP syndrome, with potential specificity to females (data not shown). From a mechanistic point of view, a protein-protein interaction analysis (string-db(dot)org/) was performed, including the ADNP-associated protein SMARCA4 (Transcription Activator BRG1)(7). TCF12 (Transcription Factor 12) and TCF21 (Transcription Factor 21), implicated in sexual behavior in humans (56), were also included in the analysis due to the marked sexual dichotomy observed in Tyr mice. The results showed a converging mechanism on AKT1, a major regulator of growth, as well as an involvement in the nervous and reproductive systems development, affected by the Tyr genotype and corrected by NAP (FIG. 5).

### Tyr Mice Show Differential Regulation of Gut Microbiota Composition:

Following, the present inventors wanted to further expand the arsenal of peripheral biomarkers. Given that the Adnp^{+/-} mouse displays sexually dichotomous microbiota signature (57), they sought to investigate whether the Tyr mouse also displays a microbiota signature and whether this could be corrected by NAP treatment. The results showed that male Tyr mice exhibit increased total eubacterial load (EubV3), Bifidobacterium genus (BIF) and Lactobacillus group (Lacto), as well as decreased loads of Enterobacteriaceae (Entero). This male phenotype was corrected with NAP treatment for the EubV3. Female Tyr mice show increased bacterial load of BIF with NAP correction (FIG. 6A). NAP had no effect on WT mice for these bacterial strains, with other varying effects on the Clostridium coccoides group (gCcoc) and Mouse intestinal Bacteroides (MIB) bacterial strains (data not shown). Following these results, the present inventors further evaluated whether these bacterial strain quantities correlated to weight and motor ability (CatWalk performance), similarly to the Adnp^{+/-} mouse (57). Indeed, several correlations were revealed, most profoundly in females, which exhibited a strong negative correlation between the BIF bacteria and weight, as well as with many CatWalk parameters. Correlations were also discovered for the Entero and gCocc strains. In addition, the relationship between bacterial groups was examined, indicating that males display a greater number of significant correlations than females, with hardly any overlap between the sexes (see PCT Publication 201907401 to Gozes).

### Proposed Mechanism:

Without being bound by theory, the present inventors proposed the following mechanism. A key target for the ADNP/NAP protective activity is the microtubule end binding proteins EB1 and EB3, critical for axonal transport and dendritic spine formation, enhancing Tau-microtubule binding and protecting against tauopathy (9, 11, 12, 21, 32). Increased mutation loads in ADNP correlate with tauopathy and NAP protects against decreased Tau-microtubule interaction in the face of ADNP mutations (21, 27). Hereinabove, in vivo evidence is provided for an Adnp Tyr mutation driving tauopathy and protection by NAP was established. Functional correlates, for example VEP, were revealed. It is important to add that the neuromuscular junction further depends on microtubule integrity associating ADNP/NAP (29, 58) and EB1/microtubules/ADNP are also key to the function of the immune synapse (57), explaining the breadth of activities of ADNP/NAP. With Alzheimer's disease being the major tauopathy, lessons from this disease teach that a driving mechanism in Alzheimer's disease may include somatic mutations (encompassing ADNP) (21), culminating on the AKT1 pathway (12, 59), as observed here. Finally, with testosterone regulated by ADNP (27), controlling the microtubule system (28) and with reduction in testosterone paralleling increased tauopathy (36), and with estrogen rescuing ADNP knockout effects in Xenopus and human models of brain development (60) sex-dependency must be taken into account (FIG. 6B).

### The Tyr Mouse Complements the Haploinsufficient Mouse Model and Mimics the Human ADNP Syndrome Patient:

Table 4 hereinbelow compares the new Tyr mouse to the human condition, as well as to the Adnp^{+/-} mouse (12). While the Adnp^{+/-} mouse shows quite extensive sexual dichotomy (9, 12, 16, 61, 62), the Tyr mouse showed even more extensive differences between the sexes. Striking examples are the dendritic spines and motor phenotypes. The Tyr mouse revealed several previously unknown phenotypes including early death, deficient syntax, sex-dependent developmental delays (e.g. eye opening) and sensory deficits (hot plate). As to gene expression patterns, splenic gene expression was far more extensive in females in the Tyr model (compared to males), while in the Adnp^{+/-} mouse model gene expression changed in both sexes to a similar degree (although not the same genes). Regardless, in both mouse lines, the regulated proteins converged on Akt1. Interestingly, gut microbiota showed similar Tyr model alterations to the Adnp^{+/-} model (57) (see PCT Publication 201907401 to Gozes). Furthermore, while Tau deposits were observed in the 11-month-old Adnp^{+/-} male mouse brain (8), in the Tyr mouse sex-specific tauopathy was observed in much younger males (2.5-month-old), coupled with VEP impairments, both ameliorated by NAP treatment and correlated with the human condition (27, 51).

TABLE 4
Summary of the findings in the Tyr mouse as compared to the Adnp^{+/-} mouse and ADNP syndrome human patients

| | | ADNP^{+/-} MOUSE | NAP | PATIENTS WITH |
|---|---|---|---|---|
| TRAIT | TYR MOUSE | (12) | EFFICACY | ADNP SYNDROME |
| COGNITIVE | Object recognition in | Morris water maze (8); | +(odor)/+(8) | All inspected thus far |
| IMPAIRMENTS, | naïve males. Social | object recognition and | | show cognitive |
| BEHAVIORAL DEFICITS | approach task in naïve | social memory, odor | | impairments (22, 104) |
| | mice of both sexes, | discrimination(9, 16) | | |
| | reared w/o handling, | | | |
| | suggesting efficacy for | | | |
| | behavioral treatments. | | | |
| | Odor discrimination | | | |
| SPEECH IMPEDIMENTS | Vocalization and | Vocalization | +/+ (syntax) | All have delays in |
| | reduced syntax | | | language acquisition (22, |
| | syllable complexity. | | | 53) and some do not speak |
| | | | | at all (15) |
| GLOBAL | Delayed air righting | Delayed air righting | +/+ | Global developmental |
| DEVELOPMENTAL | and surface righting | reflex | | delay (e.g. (15)), vision |
| DELAYS | reflexes, delayed eye | | | problems (e.g. delayed |
| | opening in females | | | visual maturation) (53) |
| SHORT STATURE | Reduced length - | Reduced length | −/+ | Short stature (24) |
| | females | | | |
| INCREASED TOUCH | Hot-plate, ear twitch | Ear twitch reflex | | Sensory processing |
| SENSITIVITY | reflex develops earlier, | develops earlier | | problems (24) |
| | auditory startle reflex | | | |
| | develops later | | | |
| MOTOR IMPEDIMENTS | Gait was significantly | Abnormal gait | +/(gait)+ | Motor |
| | impaired by the Tyr | development, reduced | | dysfunction/impaired |
| | genotype, with a more | grip strength, reduced | | development is shared by |
| | severe phenotype in | capacity in the | | the children, as part of the |
| | females. Abnormal | hanging wire test | | global developmental |
| | hanging-wire in | (males only) | | delay (15, 104) |
| | females | | | |
| SYNAPTIC | Striking sexual | Reduced synaptic | +/+ | Structural brain |
| STRUCTURAL | dichotomy: | density, increase | | abnormalities (53), |
| ALTERATIONS | Males: ~15% reduction | in immature shaft | | e.g. hippocampus (15) |
| | in CA1 hippocampal | synapses | | |
| | dendritic spines. | (hippocampus) | | |
| | Females: ~16% reduction | | | |
| | motor cortex L5 layer. | | | |
| | PSD95 volume in the | | | |
| | hippocampus, a ~25% | | | |
| | genotype decrease was | | | |
| | observed in females. | | | |
| GENE EXPRESSION | Smox, Arrb1, Adcy6, | Dysregulation of | +/+ | Dysregulation of |
| PATTERNS | Foxo3, Cpxm1- female - | splenic Iba1 (Aif1), | | lymphoblastoid IBA1 |
| | converging on AKT1 | Mtor and Cdh12 | | (AIF1), MTOR and |
| | (ADNP regulated in | Converging on AKT1 | | CDH12 |
| | human lymphoblastoids) | | | Converging on AKT1 |
| MICROBIOTA | Male specific: | Male specific: | +/+ | Microbiota is linked |
| | Eubacteriaceae (EubV3), | Eubacteriaceae (EubV3), | | with autism (105). |
| | Lactobacillus group | Lactobacillus group | | |
| | (Lacto), | (Lacto), Mouse | | |
| | Bifidobacterium genus | intestinal Bacteroides | | |
| | (BIF) and more. | (MIB) and more (57). | | |
| TAUOPATHY: | Males (stronger | Males, 11-month (8) | +/+ | 7-year-old patient brain |
| HYPERPHOSPHORYLATED | effect), 2.5-month-old | | | (postmortem)(27) |
| TAU DEPOSITS | | | | |
| VISUAL EVOKED | Visual evoked | Axonal transport, | +/+ | A biomarker for autism |
| POTENTIAL/AXONAL | potential, impaired | slower in males, | | spectrum disorders (51) |
| TRANSPORT | in males | impaired in both | | with visual impairment |
| | | sexes (9) | | previously reported in |
| | | | | ADNP syndrome patients |
| | | | | (52, 53) |

Previously unknown ADNP-associated phenotypes including corrections by NAP treatment (+) are underlined.

### REFERENCES FOR EXAMPLE 1

1. Maenner M J, Rice C E, Arneson C L, Cunniff C, Schieve L A, Carpenter L A, et al. Potential impact of DSM-5 criteria on autism spectrum disorder prevalence estimates. JAMA psychiatry. 2014; 71(3):292-300.
2. Abrahams B S, and Geschwind D H. Advances in autism genetics: on the threshold of a new neurobiology. Nature reviews Genetics. 2008; 9(5):341-55.
3. Diagnostic and statistical manual of mental disorders: DSM-5. Arlington, V A: American Psychiatric Association; 2013.
4. Bassan M, Zamostiano R, Davidson A, Pinhasov A, Giladi E, Perl 0, et al. Complete sequence of a novel protein containing a femtomolar-activity-dependent neuroprotective peptide. Journal of neurochemistry. 1999; 72(3):1283-93.
5. Zamostiano R, Pinhasov A, Gelber E, Steingart R A, Seroussi E, Giladi E, et al. Cloning and characterization of the human activity-dependent neuroprotective protein. The Journal of biological chemistry. 2001; 276(1):708-14.
6. Pinhasov A, Mandel S, Torchinsky A, Giladi E, Pittel Z, Goldsweig A M, et al. Activity-dependent neuroprotective protein: a novel gene essential for brain formation. Brain Res Dev Brain Res. 2003; 144(1):83-90.
7. Mandel S, Rechavi G, and Gozes I. Activity-dependent neuroprotective protein (ADNP) differentially interacts with chromatin to regulate genes essential for embryogenesis. Developmental biology. 2007; 303(2):814-24.
8. Vulih-Shultzman I, Pinhasov A, Mandel S, Grigoriadis N, Touloumi O, Pittel Z, et al. Activity-dependent neuroprotective protein snippet NAP reduces tau hyperphosphorylation and enhances learning in a novel transgenic mouse model. The Journal of pharmacology and experimental therapeutics. 2007; 323(2):438-49.
9. Amram N, Hacohen-Kleiman G, Sragovich S, Malishkevich A, Katz J, Touloumi O, et al. Sexual divergence in microtubule function: the novel intranasal microtubule targeting SKIP normalizes axonal transport and enhances memory. Molecular psychiatry. 2016; 21(10):1467-76.
10. Merenlender-Wagner A, Malishkevich A, Shemer Z, Udawela M, Gibbons A, Scarr E, et al. Autophagy has a key role in the pathophysiology of schizophrenia. Molecular psychiatry. 2015; 20(1):126-32.
11. Oz S, Kapitansky O, Ivashco-Pachima Y, Malishkevich A, Giladi E, Skalka N, et al. The NAP motif of activity-dependent neuroprotective protein (ADNP) regulates dendritic spines through microtubule end binding proteins. Molecular psychiatry. 2014; 19(10):1115-24.
12. Hacohen-Kleiman G, Sragovich S, Karmon G, Gao A Y L, Grigg I, Pasmanik-Chor M, et al. Activity-dependent neuroprotective protein deficiency models synaptic and developmental phenotypes of autism-like syndrome. The Journal of clinical investigation. 2018; 128(11):4956-69.
13. Schirer Y, Malishkevich A, Ophir Y, Lewis J, Giladi E, and Gozes I. Novel marker for the onset of frontotemporal dementia: early increase in activity-dependent neuroprotective protein (ADNP) in the face of Tau mutation. PloS one. 2014; 9(1):e87383.
14. Sun X, Peng X, Cao Y, Zhou Y, and Sun Y. ADNP promotes neural differentiation by modulating Wnt/beta-catenin signaling. Nat Commun. 2020; 11(1):2984.
15. Gozes I, Van Dijck A, Hacohen-Kleiman G, Grigg I, Karmon G, Giladi E, et al. Premature primary tooth eruption in cognitive/motor-delayed ADNP-mutated children. Transl Psychiatry. 2017; 7(2):e1043.
16. Malishkevich A, Amram N, Hacohen-Kleiman G, Magen I, Giladi E, and Gozes I. Activity-dependent neuroprotective protein (ADNP) exhibits striking sexual dichotomy impacting on autistic and Alzheimer's pathologies. Transl Psychiatry. 2015; 5:e501.
17. Mandel S, and Gozes I. Activity-dependent neuroprotective protein constitutes a novel element in the SWI/SNF chromatin remodeling complex. The Journal of biological chemistry. 2007; 282(47):34448-56.
18. Mosch K, Franz H, Soeroes S, Singh P B, and Fischle W. HP1 recruits activity-dependent neuroprotective protein to H3K9me3 marked pericentromeric heterochromatin for silencing of major satellite repeats. PloS one. 2011; 6(1):e15894.
19. Ostapcuk V, Mohn F, Carl S H, Basters A, Hess D, Iesmantavicius V, et al. Activity-dependent neuroprotective protein recruits HP1 and CHD4 to control lineage-specifying genes. Nature. 2018; 557(7707):739-43.
20. Kaaij L J T, Mohn F, van der Weide R H, de Wit E, and Buhler M. The ChAHP Complex Counteracts Chromatin Looping at CTCF Sites that Emerged from SINE Expansions in Mouse. Cell. 2019; 178(6):1437-51 e14.
21. Ivashko-Pachima Y, Hadar A, Grigg I, Korenkovi V, Kapitansky O, Karmon G, et al. Discovery of autism/intellectual disability somatic mutations in Alzheimer's brains: mutated ADNP cytoskeletal impairments and repair as a case study. Molecular psychiatry. 2019.
22. Helsmoortel C, Vulto-van Silfhout A T, Coe B P, Vandeweyer G, Rooms L, van den Ende J, et al. A SWI/SNF-related autism syndrome caused by de novo mutations in ADNP. Nat Genet. 2014; 46(4):380-4.
23. Larsen E, Menashe I, Ziats M N, Pereanu W, Packer A, and Banerjee-Basu S. A systematic variant annotation approach for ranking genes associated with autism spectrum disorders. Molecular autism. 2016; 7:44.
24. Van Dijck A, Vulto-van Silfhout A T, Cappuyns E, van der Werf I M, Mancini G M, Tzschach A, et al. Clinical Presentation of a Complex Neurodevelopmental Disorder Caused by Mutations in ADNP. Biol Psychiatry. 2019; 85(4):287-97.
25. Gozes I, Patterson M C, Van Dijck A, Kooy R F, Peeden J N, Eichenberger J A, et al. The Eight and a Half Year Journey of Undiagnosed A D: Gene Sequencing and Funding of Advanced Genetic Testing Has Led to Hope and New Beginnings. Frontiers in endocrinology. 2017; 8:107.
26. Gozes I, Yeheskel A, and Pasmanik-Chor M. Activity-dependent neuroprotective protein (ADNP): a case study for highly conserved chordata-specific genes shaping the brain and mutated in cancer. Journal of Alzheimer's disease: JAD. 2015; 45(1):57-73.
27. Grigg I, Ivashko-Pachima Y, Hait T A, Korenkovi V, Touloumi O, Lagoudaki R, et al. Tauopathy in the young autistic brain: novel biomarker and therapeutic target. Transl Psychiatry. 2020; 10(1):228.
28. Butler R, Leigh P N, and Gallo J M. Androgen-induced up-regulation of tubulin isoforms in neuroblastoma cells. Journal of neurochemistry. 2001; 78(4):854-61.
29. Kapitansky O, Karmon G, Sragovich S, Hadar A, Shahoha M, Jaljuli I, et al. Single Cell ADNP Predictive of Human Muscle Disorders: Mouse Knockdown Results in Muscle Wasting. Cells. 2020; 9(10).
30. Gozes I, Bassan M, Zamostiano R, Pinhasov A, Davidson A, Giladi E, et al. A novel signaling molecule for neuropeptide action: activity-dependent neuroprotective protein. Annals of the New York Academy of Sciences. 1999; 897:125-35.
31. Divinski I, Mittelman L, and Gozes I. A femtomolar acting octapeptide interacts with tubulin and protects astrocytes against zinc intoxication. The Journal of biological chemistry. 2004; 279(27):28531-8.
32. Ivashko-Pachima Y, Sayas C L, Malishkevich A, and Gozes I. ADNP/NAP dramatically increase microtubule end-binding protein-Tau interaction: a novel avenue for protection against tauopathy. Molecular psychiatry. 2017; 22(9):1335-44.
33. Pascual M, and Guerri C. The peptide NAP promotes neuronal growth and differentiation through extracellular signal-regulated protein kinase and Akt pathways, and protects neurons co-cultured with astrocytes damaged by ethanol. Journal of neurochemistry. 2007; 103(2):557-68.
34. Patel S, Roncaglia P, and Lovering R C. Using Gene Ontology to describe the role of the neurexin-neuroligin-SHANK complex in human, mouse and rat and its relevance to autism. BMC bioinformatics. 2015; 16:186.
35. Jouroukhin Y, Ostritsky R, Assaf Y, Pelled G, Giladi E, and Gozes I. NAP (davunetide) modifies disease progression in a mouse model of severe neurodegeneration: protection against impairments in axonal transport. Neurobiology of disease. 2013; 56:79-94.
36. Sundermann E E, Panizzon M S, Chen X, Andrews M, Galasko D, Banks S J, et al. Sex differences in Alzheimer's-related Tau biomarkers and a mediating effect of testosterone. Biology of sex differences. 2020; 11(1):33.
37. Malishkevich A, Marshall G A, Schultz A P, Sperling R A, Aharon-Peretz J, and Gozes I. Blood-Borne Activity-Dependent Neuroprotective Protein (ADNP) is Correlated with Premorbid Intelligence, Clinical Stage, and Alzheimer's Disease Biomarkers. Journal of Alzheimer's disease: JAD. 2016; 50(1):249-60.
38. Marcucio R S, Young N M, Hu D, and Hallgrimsson B. Mechanisms that underlie co-variation of the brain and face. Genesis. 2011; 49(4):177-89.
39. Luchetti A, Oddi D, Lampis V, Centofante E, Felsani A, Battaglia M, et al. Early handling and repeated cross-fostering have opposite effect on mouse emotionality. Front Behav Neurosci. 2015; 9:93.
40. Bartolomucci A, Gioiosa L, Chirieleison A, Ceresini G, Parmigiani S, and Palanza P. Cross fostering in mice: behavioral and physiological carry-over effects in adulthood. Genes Brain Behav. 2004; 3(2):115-22.
41. Lu L, Mamiya T, Lu P, Niwa M, Mouri A, Zou L B, et al. The long-lasting effects of cross-fostering on the emotional behavior in ICR mice. Behav Brain Res. 2009; 198(1):172-8.
42. Silverman J L, Yang M, Lord C, and Crawley J N. Behavioural phenotyping assays for mouse models of autism. Nat Rev Neurosci. 2010; 11(7):490-502.
43. Stefanatos G A, and Baron I S. The ontogenesis of language impairment in autism: a neuropsychological perspective. Neuropsychol Rev. 2011; 21(3):252-70.
44. Ehret G. Infant rodent ultrasounds—a gate to the understanding of sound communication. Behav Genet. 2005; 35(1):19-29.
45. Yin X, Chen L, Xia Y, Cheng Q, Yuan J, Yang Y, et al. Maternal Deprivation Influences Pup Ultrasonic Vocalizations of C57BL/6J Mice. PloS one. 2016; 11(8):e0160409.
46. Grimsley J M, Monaghan J J, and Wenstrup J J. Development of social vocalizations in mice. PloS one. 2011; 6(3):e17460.
47. Coffey K R, Marx R G, and Neumaier J F. DeepSqueak: a deep learning-based system for detection and analysis of ultrasonic vocalizations. Neuropsychopharmacology. 2019; 44(5):859-68.
48. Phillips M, and Pozzo-Miller L. Dendritic spine dysgenesis in autism related disorders. Neurosci Lett. 2015; 601:30-40.
49. Sragovich S, Malishkevich A, Piontkewitz Y, Giladi E, Touloumi O, Lagoudaki R, et al. The autism/neuroprotection-linked ADNP/NAP regulate the excitatory glutamatergic synapse. Transl Psychiatry. 2019; 9(1):2.
50. Stothart G, Kazanina N, Naatanen R, Haworth J, and Tales A. Early visual evoked potentials and mismatch negativity in Alzheimer's disease and mild cognitive impairment. Journal of Alzheimer's disease: JAD. 2015; 44(2):397-408.
51. Siper P M, Zemon V, Gordon J, George-Jones J, Lurie S, Zweifach J, et al. Rapid and Objective Assessment of Neural Function in Autism Spectrum Disorder Using Transient Visual Evoked Potentials. PloS one. 2016; 11(10):e0164422.
52. Gale M J, Titus H E, Harman G A, Alabduljalil T, Dennis A, Wilson J L, et al. Longitudinal ophthalmic findings in a child with Helsmoortel-Van der Aa Syndrome. Am J Ophthalmol Case Rep. 2018; 10:244-8.
53. Gozes I, Helsmoortel C, Vandeweyer G, Van der Aa N, Kooy F, and Sermone S B. The Compassionate Side of Neuroscience: Tony Sermone's Undiagnosed Genetic Journey—ADNP Mutation. Journal of molecular neuroscience: MN. 2015; 56(4):751-7.
54. Breen M S, Garg P, Tang L, Mendonca D, Levy T, Barbosa M, et al. Episignatures Stratifying Helsmoortel-Van Der Aa Syndrome Show Modest Correlation with Phenotype. Am J Hum Genet. 2020; 107(3):555-63.
55. Hopkins P C, Sainz-Fuertes R, and Lovestone S. The impact of a novel apolipoprotein E and amyloid-beta protein precursor-interacting protein on the production of amyloid-beta. Journal of Alzheimer's disease: JAD. 2011; 26(2):239-53.
56. Ganna A, Verweij K J H, Nivard M G, Maier R, Wedow R, Busch A S, et al. Large-scale GWAS reveals insights into the genetic architecture of same-sex sexual behavior. Science. 2019; 365(6456).
57. Kapitansky O, Giladi E, Jaljuli I, Bereswill S, Heimesaat M M, and Gozes I. Microbiota changes associated with ADNP deficiencies: rapid indicators for NAP (CP201) treatment of the ADNP syndrome and beyond. Journal of neural transmission. 2020; 127(2):251-63.
58. Kapitansky O, Sragovich S, Jaljuli I, Hadar A, Giladi E, and Gozes I. Age and Sex-Dependent ADNP Regulation of Muscle Gene Expression Is Correlated with Motor Behavior: Possible Feedback Mechanism with PACAP. Int J Mol Sci. 2020; 21(18).
59. Soheili-Nezhad S, van der Linden R J, Olde Rikkert M, Sprooten E, and Poelmans G. Long genes are more frequently affected by somatic mutations and show reduced expression in Alzheimer's disease: Implications for disease etiology. Alzheimers Dement. 2020.
60. Willsey H R, Exner C R T, Xu Y, Everitt A, Sun N, Wang B, et al. Parallel in vivo analysis of large-effect autism genes implicates cortical neurogenesis and estrogen in risk and resilience. Neuron. 2021.
61. Gozes I. Sexual divergence in activity-dependent neuroprotective protein impacting autism, schizophrenia, and Alzheimer's disease. Journal of neuroscience research. 2017; 95(1-2):652-60.
62. Sragovich S, Ziv Y, Vaisvaser S, Shomron N, Hendler T, and Gozes I. The autism-mutated ADNP plays a key role in stress response. Transl Psychiatry. 2019; 9(1):235.
63. Wohr M, Silverman J L, Scattoni M L, Turner S M, Harris M J, Saxena R, et al. Developmental delays and reduced pup ultrasonic vocalizations but normal sociability in mice lacking the postsynaptic cell adhesion protein neuroligin2. Behav Brain Res. 2013; 251:50-64.
64. Findlater G S, McDougall R D, and Kaufman M H. Eyelid development, fusion and subsequent reopening in the mouse. J Anat. 1993; 183 (Pt 1):121-9.
65. Mollinedo P, Kapitansky O, Gonzalez-Lamuno D, Zaslavsky A, Real P, Gozes I, et al. Cellular and animal models of skin alterations in the autism-related ADNP syndrome. Sci Rep. 2019; 9(1):736.
66. Tomchek S D, and Dunn W. Sensory processing in children with and without autism: a comparative study using the short sensory profile. Am J Occup Ther. 2007; 61(2):190-200.
67. Schaffler M D, Middleton L J, and Abdus-Saboor I. Mechanisms of Tactile Sensory Phenotypes in Autism: Current Understanding and Future Directions for Research. Curr Psychiatry Rep. 2019; 21(12):134.
68. Siemann J K, Veenstra-VanderWeele J, and Wallace M T. Approaches to Understanding Multisensory Dysfunction in Autism Spectrum Disorder. Autism Res. 2020.
69. Vieira W F, Malange K F, de Magalhaes S F, Dos Santos G G, de Oliveira A L R, da Cruz-Hofling M A, et al. Gait analysis correlates mechanical hyperalgesia in a model of streptozotocin-induced diabetic neuropathy: A CatWalk dynamic motor function study. Neurosci Lett. 2020; 736:135253.
70. Vrinten D H, and Hamers F F. ‘CatWalk’ automated quantitative gait analysis as a novel method to assess mechanical allodynia in the rat; a comparison with von Frey testing. Pain. 2003; 102(1-2):203-9.
71. Chen Y J, Cheng F C, Sheu M L, Su H L, Chen C J, Sheehan J, et al. Detection of subtle neurological alterations by the Catwalk X T gait analysis system. J Neuroeng Rehabil. 2014; 11:62.
72. Arnett A B, Beighley J S, Kurtz-Nelson E C, Hoekzema K, Wang T, Bernier R A, et al. Developmental Predictors of Cognitive and Adaptive Outcomes in Genetic Subtypes of Autism Spectrum Disorder. Autism Res. 2020; 13(10):1659-69.
73. Agarwalla S, Arroyo N S, Long N E, O'Brien W T, Abel T, and Bandyopadhyay S. Male-specific alterations in structure of isolation call sequences of mouse pups with 16p11.2 deletion. Genes Brain Behav. 2020; 19(7):e12681.
74. Simola N. Rat Ultrasonic Vocalizations and Behavioral Neuropharmacology: From the Screening of Drugs to the Study of Disease. Curr Neuropharmacol. 2015; 13(2):164-79.
75. Simola N, and Costa G. Emission of categorized 50-kHz ultrasonic vocalizations in rats repeatedly treated with amphetamine or apomorphine: Possible relevance to drug-induced modifications in the emotional state. Behav Brain Res. 2018; 347:88-98.
76. Sartucci F, Borghetti D, Bocci T, Murri L, Orsini P, Porciatti V, et al. Dysfunction of the magnocellular stream in Alzheimer's disease evaluated by pattern electroretinograms and visual evoked potentials. Brain Res Bull. 2010; 82(3-4):169-76.
77. Risacher S L, Wudunn D, Pepin S M, MaGee T R, McDonald B C, Flashman L A, et al. Visual contrast sensitivity in Alzheimer's disease, mild cognitive impairment, and older adults with cognitive complaints. Neurobiol Aging. 2013; 34(4):1133-44.
78. Rizzo M, Anderson S W, Dawson J, and Nawrot M. Vision and cognition in Alzheimer's disease. Neuropsychologia. 2000; 38(8):1157-69.
79. Sayorwan W, Phianchana N, Permpoonputtana K, and Siripornpanich V. A Study of the Correlation between VEP and Clinical Severity in Children with Autism Spectrum Disorder. Autism Res Treat. 2018; 2018:5093016.
80. Brunet A, Bonni A, Zigmond M J, Lin M Z, Juo P, Hu L S, et al. Akt promotes cell survival by phosphorylating and inhibiting a Forkhead transcription factor. Cell. 1999; 96(6):857-68.
81. Lehtinen M K, Yuan Z, Boag P R, Yang Y, Villen J, Becker E B, et al. A conserved MST-FOXO signaling pathway mediates oxidative-stress responses and extends life span. Cell. 2006; 125(5):987-1001.
82. Band A M, and Kuismanen E. Localization of plasma membrane t-SNAREs syntaxin 2 and 3 in intracellular compartments. BMC Cell Biol. 2005; 6(1):26.
83. Bennett M K, Garcia-Arraras J E, Elferink L A, Peterson K, Fleming A M, Hazuka C D, et al. The syntaxin family of vesicular transport receptors. Cell. 1993; 74(5):863-73.
84. Humeau J, Leduc M, Cerrato G, Loos F, Kepp O, and Kroemer G. Phosphorylation of eukaryotic initiation factor-2alpha (eIF2alpha) in autophagy. Cell Death Dis. 2020; 11(6):433.
85. Park J S, Lee J, Jung E S, Kim M H, Kim I B, Son H, et al. Brain somatic mutations observed in Alzheimer's disease associated with aging and dysregulation of tau phosphorylation. Nat Commun. 2019; 10(1):3090.
86. Liu Z, Mao X, Dan Z, Pei Y, Xu R, Guo M, et al. Gene variations in autism spectrum disorder are associated with alteration of gut microbiota, metabolites and cytokines. Gut Microbes. 2021; 13(1):1-16.
87. Xu M, Xu X, Li J, and Li F. Association Between Gut Microbiota and Autism Spectrum Disorder: A Systematic Review and Meta-Analysis. Front Psychiatry. 2019; 10:473.
88. Bostanciklioglu M. The role of gut microbiota in pathogenesis of Alzheimer's disease. J Appl Microbiol. 2019; 127(4):954-67.
89. Klingelhoefer L, and Reichmann H. Pathogenesis of Parkinson disease—the gut-brain axis and environmental factors. Nat Rev Neurol. 2015; 11(11):625-36.
90. Goehler L E, Gaykema R P, Opitz N, Reddaway R, Badr N, and Lyte M. Activation in vagal afferents and central autonomic pathways: early responses to intestinal infection with Campylobacter jejuni. Brain Behav Immun. 2005; 19(4):334-44.
91. Heimesaat M M, Mousavi S, Klove S, Genger C, Weschka D, Giladi E, et al. Immune-modulatory Properties of the Octapeptide NAP in Campylobacter jejuni Infected Mice Suffering from Acute Enterocolitis. Microorganisms. 2020; 8(6).
92. Escher U, Giladi E, Dunay I R, Bereswill S, Gozes I, and Heimesaat M M. Anti-inflammatory Effects of the Octapeptide NAP in Human Microbiota-Associated Mice Suffering from Subacute Ileitis. Eur J Microbiol Immunol (Bp). 2018; 8(2):34-40.
93. Heimesaat M M, Giladi E, Kuhl A A, Bereswill S, and Gozes I. The octapetide NAP alleviates intestinal and extra-intestinal anti-inflammatory sequelae of acute experimental colitis. Peptides. 2018; 101:1-9.
94. Furman S, Hill J M, Vulih I, Zaltzman R, Hauser J M, Brenneman D E, et al. Sexual dimorphism of activity-dependent neuroprotective protein in the mouse arcuate nucleus. Neurosci Lett. 2005; 373(1):73-8.
95. Hacohen-Kleiman G, Moaraf S, Kapitansky O, and Gozes I. Sex- and Region-Dependent Expression of the Autism-Linked ADNP Correlates with Social- and Speech-Related Genes in the Canary Brain. Journal of molecular neuroscience: MN. 2020; 70(11):1671-83.
96. Kapitansky O, and Gozes I. ADNP differentially interact with genes/proteins in correlation with aging: a novel marker for muscle aging. GeroScience. 2019; 41(3):321-40.
97. Ybot-Gonzalez P, Cogram P, Gerrelli D, and Copp A J. Sonic hedgehog and the molecular regulation of mouse neural tube closure. Development. 2002; 129(10):2507-17.
98. Gozes I. The ADNP Syndrome and CP201 (NAP) Potential and Hope. Front Neurol. 2020; 11:608444.
99. Henao-Mejia J, Williams A, Rongvaux A, Stein J, Hughes C, and Flavell R A. Generation of Genetically Modified Mice Using the CRISPR-Cas9 Genome-Editing System. Cold Spring Harb Protoc. 2016; 2016(2):pdb prot090704.
100. Theotokis P, Lourbopoulos A, Touloumi O, Lagoudaki R, Kofidou E, Nousiopoulou E, et al. Time course and spatial profile of Nogo-A expression in experimental autoimmune encephalomyelitis in C57BL/6 mice. J Neuropathol Exp Neurol. 2012; 71(10):907-20.
101. Ivashko-Pachima Y, Maor-Nof M, and Gozes I. NAP (davunetide) preferential interaction with dynamic 3-repeat Tau explains differential protection in selected tauopathies. PloS one. 2019; 14(3):e0213666.
102. Wu H, Xiong W C, and Mei L. To build a synapse: signaling pathways in neuromuscular junction assembly. Development. 2010; 137(7):1017-33.
103. Martin-Cofreces N B, Baixauli F, Lopez M J, Gil D, Monjas A, Alarcon B, et al. End-binding protein 1 controls signal propagation from the T cell receptor. The EMBO journal. 2012; 31(21):4140-52.
104. Gozes I. Neuroprotection in Alzheimer's Disease. Academic Press/Eslsevier; 2017:253-70.
105. Svoboda E. Could the gut microbiome be linked to autism? Nature. 2020; 577(7792):S14-S5.

### Example 2

### Adnp and Nap Comprise SH3 Domain Binding Site, Indicating Therapeutic Effect on Multiple Cytoskeleton—Related Diseases

### Materials and Methods

Plasmid construction—Protein expressing plasmids were constructed as previously described based on pEGFP-C1 backbone and express full-length ADNP or the following truncated ADNP proteins: p.Glu830synfs*83, p.Lys408Valfs*31, p.Ser404* (as before [31]). Inserts of cDNA carrying unique mutations were obtained from mRNA extracted from patient-derived lymphoblastoid cell lines and cDNA with full-length human ADNP was obtained from a control lymphoblastoid cell line with no mutation [77]. Protein expressions were verified by fluorescent imaging and immunoblotting analysis as before [31].

Cell culture—Mouse neuroblastoma NIE-115 cells were maintained in Dulbecco's modified Eagle's medium, 10% fetal bovine serum, 2 mM glutamine and 100 U ml—1 penicillin, 100 mg ml—1 streptomycin (Biological Industries, Beit Haemek, Israel). The cells were incubated in 95% air/5% CO2 in a humidified incubator at 37° C.

Cell differentiation and co-transfection of overexpression plasmids—Cultured NIE-115 cells were plated on 35 mm dishes (81156, 60μ-Dish, Ibidi, Martinsried, Germany) at a concentration of 1.25×10⁴ cells per dish and then were differentiated with reduced fetal bovine serum (2%) and DMSO (1.25%) containing medium for 5 days before transfection. 48 hrs before experiments N1E-115 cells were co-transfected with 1 μg EB3-RFP or mCherry-Tau (3R) plasmid with or without 2 μg of plasmids, coding to GFP conjugated to full-length human ADNP, or ADNP carried mutations. 1:3 ratios between total amount of transfected DNA and transfection reagent were used according to manufacture guidance (Lipofectamine2000, Thermo Fisher Scientific, Waltham, MA, USA) in all subsequent experiments.

Live Imaging—48 hrs after transfection, differentiated NIE-115 cells (expressing EB3-RFP with or without GFP-conjugated to full-length ADNP or mutated ADNP) were incubated at 37° C. with a 5% CO2/95% air mixture in a thermostatic chamber placed on the stage of a Leica TCS SP5 confocal microscope (objective 100× (PL Apo) oil immersion, NA 1.4). Time-lapse images were automatically captured 3 sec during 1 min, using the Leica LAS AF software. After imaging all samples were also treated with NAP (SEQ ID NO: 2) or SKIP (SEQ ID NO: 21) in final concentrations of 10⁻¹² M, and after 4 hrs time-lapse imaging was done again under the same conditions. Data was collected and analyzed by Imaris software.

Fluorescence recovery after photobleaching (FRAP)—Differentiated NIE-115 cells were transfected with mCherry-Tau with or without GFP-conjugated to full-length ADNP or mutated ADNP, and imaged 48 hrs after transfection. An ROI for photobleaching was drowned in the proximal cell branches. mCherry-Tau was bleached with a 587 nm argon laser, and fluorescence recovery was collected within wavelengths of 610-650 nm. 80 images were taken every 0.74 sec immediately after bleaching. After FRAP imaging all samples were also treated with NAP in final concentrations of 10⁻¹² M, and after 4 hrs time-lapse imaging was done again under the same conditions. FRAP imaging for assessment difference between NAP and SKIP (SEQ ID NO: 21) activity was performed after an hour of ZnCl₂ treatment (400 μM final concentration; Sigma, Rehovot, Israel) with or without NAP/SKIP (10⁻¹² M final concentration). Fluorescence signals were quantified with Fiji [81], obtained data were normalized with easyFRAP [82, 83] and FRAP recovery curves were fitted by a one-phase exponential association function by GraphPad Prism6 (GraphPad Software, Inc., La Jolla, CA, USA) while samples with R²<0.9 were excluded.

Polymerized vs. soluble tubulin assay—To quantify tubulin polymerization and Tau-MT association, a simple assay was performed as previously described [19, 25]. The cells grown to confluence in six-well plates were washed with MT buffer (80 mM PIPES pH 6.8, 1 mM MgCl², 2 mM EGTA, 5% Glycerol) and lysed at room temperature for 5 min through centrifugation (300 g), with 150 μl of MT buffer with 0.5% Triton X-100 to extract soluble (cytosolic) tubulin (S). The pelleted cells were rinsed once again with an equal volume of modified RIPA lysis buffer (50 mM Tris-HCL pH 7.4, 150 mM NaCl, 2 mM EGTA, 1% Triton X-100, 0.1% SDS, 0.1% sodium Deoxycholate) to collect the polymerized (cytoskeletal) tubulin (P). The cytosolic and cytoskeletal fractions were each mixed with sample buffer and heated at 95° C. for 5 min. An equal volume of each fraction was analyzed by immunoblotting with Tau, tubulin and actin antibodies, and the results following ECL development were quantified by densitometry (GelQuant.NET software provided by biochemlabsolutions(dot)com).

Statistical analysis—The data is presented as the mean±S.E.M., from at least three independent experiments. Statistical analysis of the data was performed using Two Way ANOVA by SigmaPlot 11 (Systat Software, Inc., San Jose, CA, USA) or One Way ANOVA by IBM SPSS 23 (IBM, Armonk, NY, USA). Tukey post hoc test was performed for all pairwise multiple comparison procedures.

### Results

### Expression of ASD-Associated ADNP Truncated Proteins Impairs EB3 Activity at MT Plus-Ends and NAP Restores EB3 Action:

To reveal the effect of ADNP mutations implicated in ADNP syndrome on the microtubular cytoskeleton four plasmids expressing proteins conjugated to green fluorescent protein (GFP) were constructed. The three plasmids expressed the following truncated forms of ADNP: p.Glu830synfs*83, p.Lys408Valfs*31, p.Ser404* (as described in [30, 31]). Backbone plasmid pEGFP-C1 and full-length ADNP-containing plasmid were used as controls in live imaging experiments. Expression of the designed ADNP forms was verified by fluorescent imaging and Western Blot analysis as before [30, 31].

To evaluate the effect of ADNP truncated proteins on MT dynamics time-lapse imaging of EB3 comet-like structures formed by RFP-tagged EB3 proteins that bind to MT plus-ends was performed. Two parameters of EB3 activity were used to assess MT dynamics—track length and track speed of EB3 comets, which reflect the length of freshly growing MTs and the speed of MT assembly, respectively. Furthermore, the present inventors aimed to determine whether NAP (10⁻¹² M for 4 hrs) could prevent the deleterious effect of ADNP mutations.

Overexpression of full-length ADNP significantly increased EB3 comet track length and did not have any impact on the speed of EB3 comets, while NAP treatment did not further influence the effect of full-length ADNP (FIGS. 7A-C). Expression of ADNP p.Ser404* truncated form significantly decreased EB3 comet track lengths, compared to either full-length ADNP or control plasmid. However, truncated ADNP p.Glu830synfs*83 showed a significant reduction of EB3 track length only in comparison to full-length ADNP (FIGS. 7A-B). Evaluation of the second parameter of EB3 activity—comet speed indicated a significant effect of ADNP p.Glu830synfs*83 and p.Ser404* compared to the both full-length ADNP and control (FIG. 7C). Interestingly, the expression of p.Lys408Valfs*31 ADNP form did not have any impact on EB3 activity in comparison to neither full-length ADNP nor control (FIGS. 7A-C). NAP treatment significantly augmented the speed and track length of the EB3 comets of p.Glu830synfs*83 and p.Ser404* groups and comet speed of p.Lys408Valfs*31 (FIGS. 7A-C).

Expression of ASD-Associated ADNP Truncated Proteins Diminishes Tau Association with MTs and NAP Recovers Tau-MT Interactions:

Next, the effect of full-length/truncated ADNP forms with or without NAP treatment on Tau-MT interaction was examined by fluorescence recovery after photobleaching (FRAP) (FIGS. 8A-C). FRAP was performed with mCherry-tagged Tau protein (FIG. 8A). The plateau of fluorescence recovery within a photo-bleached region of interest (ROI) determines the immobile fractions of mCherry-Tau bleached molecules, which do not release binding sites on MTs for incoming un-bleached mCherry-Tau proteins and thus do not contribute to the fluorescence recovery. Therefore, the immobile mCherry-Tau fraction reflects the rate of Tau association with MTs. The present inventors observed that overexpression of full-length ADNP resulted in a minor insignificant reduction of immobile mCherry-Tau fraction in comparison to control plasmid (FIGS. 8A-C). Expression of p.Ser404* significantly attenuated Tau association with MTs, compared to the both full-length ADNP and control, and the effect of p.Glu830synfs*83 was found to be significantly different only in comparison to control, but not to full-length ADNP (FIGS. 8A-C). NAP treatment significantly increased Tau-MT interaction within p.Glu830synfs*83 and p.Ser404* groups. However, as in the previous experiment with EB3 activity p.Lys408Valfs*31 did not impair Tau-MT association (FIGS. 8A-C).

Frameshift Sequence of p.Lys408Valfs*31 Contains SH3-Binding Motif which Restores ADNP Activity on MTs:

ADNP p.Lys408Valsf*31 truncated form exhibited moderate non-significant effect on MT assembly and Tau-MT interaction compared to other upstream and down-stream mutations including results of ADNP p.Arg730* and p.Tyr719* truncated forms obtained from the previously published data [30, 31] (FIGS. 9A-B). Elm analysis [33] of p.Lys408Valfs*31 amino acid tail which differs from p.Ser404* (FIG. 9C) indicated recreated Src homology 3 (SH3) binding site among others Elm-predicted motifs.

Protein-protein interactions, mediated by SH3 domain-ligand association, are involved in a wide variety of biological processes ranging from enzyme activation/inactivation by intramolecular interactions, alteration of cellular concentration/localization of signaling components, and mediation of multi-protein complex assembly (Moarefi et al. Nature 1997, 385: 650-653).

Tau protein's SH3-binding domain associates with Tau kinases and modulates Tau phosphorylation and MT interaction [34]. Thus, the present inventors aimed to reveal whether the observed decreased impact of ADNP p.Lys408Valsf*31 expression on MT-Tau interaction and EB3 activity was mediated by predicted SH3-binding domain. For this purpose, the SH3-binding domain on ADNP p.Lys408Valsf*31 plasmid was broken by substitution of one amino-acid with site-directed mutagenesis (SDM). Expression of new ADNP p.Lys408Valsf*31SDM plasmid showed a significant decrease in EB3 activity by both tested parameters—EB3 track length and comet speed compared to results obtained from expression of control plasmid, full-length ADNP and ADNP p.Lys408Valsf*31 plasmid without SDM (FIGS. 10A-C) and NAP treatment recovered EB3 activity to the control level (FIGS. 10A-C).

FRAP analysis indicated attenuated Tau immobile fraction following ADNP p.Lys408Valsf*31SDM expression which was found a statistically significant in comparison to control, full-length ADNP, and ADNP p.Lys408Valsf*31 without SDM (FIGS. 10D-F). Adverse effect of ADNP p.Lys408Valsf*31SDM expression on Tau-MT interaction was prevented by NAP treatment (FIGS. 10D-F).

### NAP SH3-Binding Motif Increases the Protective Activity of NAP Against Tau-MT Disassociation:

Elm analysis of NAP sequence indicated SH3-binding motif overlapped with SIP EB-binding site [24] (Table 5 hereinbelow).

TABLE 5
ELM analysis of NAPVSIPQ (SEQ ID NO: 2, NAP) sequence, predicting
a SH3-binding site (LIG_SH3_3) within the sequence.

| | Matched | | | |
|---|---|---|---|---|
| Elm Name | Sequence | Positions | Elm Description | Probability |
| DEG | NA | 1-2 [A] | N-terminal motif | 1.65E−04 |
| Nend_UB | | | that initiates protein | |
| Rbox_3 | | | degradation by | |
| | | | binding to the | |
| | | | UBR-box of N- | |
| | | | recognins. | |
| LIG | NAPVSIP | 1-7 [A] | This is the motif | 1.32E−02 |
| SH3_3 | | | recognized by those | |
| | | | SH3 domains with | |
| | | | a non-canonical | |
| | | | class I recognition | |
| | | | specificity | |

Interestingly, ELM analysis predicted multiple SH3 domain binding sites on ADNP coupled to an actin binding site, which may offer another site of indirect interaction between SHANK3 and ADNP, with SHANK3 activity dependent of actin and ADNP on microtubules, involving multiple cytoskeleton dependent disease, with NAP correction.

The effect of NAP on MT dynamics and Tau-MT interaction in comparison to SKIP (SEQ ID NO: 22) peptide which consists of SxIP EB-associating site but lacks SH3-binding motif, was evaluated. Time-lapse imaging tracking the growth of individual MTs with RFP-tagged EB3 proteins showed that NAP and SKIP treatments (10⁻¹² M, 4 hrs) resulted in increased EB3 track length and speed compared to non-treated control and no significant differences were observed between effects of NAP and SKIP (FIGS. 11A-B).

Further, a FRAP assay was performed using zinc as Tau-MT dissociation agent [19, 25] to assess NAP and SKIP protective activity on the interaction of Tau with MTs (FIGS. 11C-E). The results showed that extracellular zinc (400 μM, 1 h) significantly attenuated Tau immobile fraction in comparison to non-treated control while treatment with SKIP (10⁻¹² M, 1 h) prevented zinc adverse effect. Exposure to zinc together with NAP (10⁻¹² M, 1 h) exhibited excessive impact of NAP on Tau-MT interaction, which was found statistically significant in comparison to zinc treatment with and without SKIP and non-treated control (FIGS. 11C-E).

It is known that in the presence of extracellular zinc NAP protects MT against degradation by promoting Tau binding to the MT shaft [19, 25]. Polymerized vs. soluble tubulin assay was effected to compare protective activities of NAP and SKIP against MT degradation and Tau-MT dissociation (FIGS. 11F-G). After treatments with zinc (400 μM, 4 h) with and without NAP or SKIP (10⁻¹² M, 4 h) the cellular-tubulin pool was separated into polymerized and soluble tubulin fractions. The amount of Tau, tubulin, and actin was identified by immunoblotting (FIG. 11F), followed by densitometric quantification (FIG. 11G). Zinc treatment significantly increased the immunoreactivity of Tau and tubulin in the soluble fraction in comparison to non-treated control, indicating Tau release from MTs and MTs degradation (FIGS. 11F-G). Polymerized tubulin was protected against zinc-induced decay by both NAP and SKIP treatments, but only NAP exhibited protection against Tau dissociation from MTs (FIGS. 11F-G). No changes were observed in the amount of actin protein in soluble tubulin fractions following different treatments (FIG. 1F).

### NAP Protected Against SHANK3-Related Mutation, the Autism Model of Phelan McDermid Syndrome:

SH3 and multiple ankyrin repeat domains 3 (Shank3), also known as proline-rich synapse-associated protein 2 (ProSAP2), is a protein that in humans is encoded by the SHANK3 gene on chromosome 22. Mutations in SHANK3 are causative for the Phelan McDermid syndrome. Regardless of previous findings of SHANK3 autistic mice do not present tauopathy ([30]), the present inventors tested a potential effect of NAP in a SHANK3 mouse, given the SH3 site(s).

To this end, mice with the ASD-linked InsG3680 mutation [35] were used. Two behavioral assessments were performed on make mice:
1) Open field behavior attesting to anxiety and depression (Open field behavioral assessment technology was previously described, e.g. [36]); and
2) Social recognition behavior, related to autistic traits (the social recognition test was previously described, e.g. [38, 39].

Open field behavior showed a dramatic reduction in cumulative duration in the center, indicative of anxious/depressive behavior and this was significantly increased by NAP treatment (once daily, five days a week for a month, intranasal administration, as described in Example 1 hereinabove) (FIG. 12). This surprising finding is of importance as there is a psychiatric illness and regression in individuals with Phelan-McDermid syndrome [37].

In the social recognition test, the results measured both time exploring and frequency exploring the mouse or cup, either directly or in the chamber where the mouse or cup only are present (FIG. 13). The results clearly showed an indifferent behavior of the WT littermates as well as of the SHANK3 mutated mice, while the mutation resulted in a significant reduction in mouse exploration, i.e. autistic behavior, this was coupled to normalization of behavior in the NAP-treated SHANK3 mouse model, with significance preference of the mouse over the cup (object) in the presence of NAP, suggesting an enhancement of autistic-related social behavior, by NAP.

Repetitive behavior, a key autistic feature, was also evaluated by measuring grooming frequency and duration. Average grooming duration, calculated by the cumulative duration of grooming divided by the frequency of such behavior, indicated that sham-treated SHANK3 mice showed higher values than did their WT counterparts (data not shown). The sham-treated mice also showed higher grooming frequency than did NAP-treated SHANK3 mice (data not shown).

Similarly, as described for the ADNP syndrome Eubacteriaceae (EubV3) [40] showed a trend to change as a consequence of the SHANK3 mutation, and this trend was not observed in the treated mice (data not shown).

Noteworthy, the effects on social recognition (i.e. preference of the mouse, rather than the cup=object chamber) by NAP were also observed in to a general idiopathic autism mouse, the BTBR mouse [42] (FIG. 14).

### SUMMARY OF THE RESULTS

De novo heterozygous mutations in ADNP reproduced the autistic ADNP syndrome. ADNP mutations impair microtubule (MT) function, essential for synaptic activity. The ADNP MT-associating fragment NAPVSIPQ (NAP) (SEQ ID NO 2) contains an MT end-binding protein interacting domain, SxIP (mimicking the active-peptide, SKIP, SEQ ID NO 21). The inventors hypothesized that not all ADNP mutations are similarly deleterious and that the NAPV portion of NAPVSIPQ is biologically active. Using the eukaryotic linear motif (ELM) resource, the inventors identified a Src homology 3 (SH3) domain-ligand association site in NAP responsible for controlling signaling pathways regulating the cytoskeleton, namely NAPVSIP (SEQ ID NO: 2). Altogether, the inventors mapped multiple SH3-binding sites in ADNP. Comparisons of the effects of ADNP mutations p.Glu830synfs*83, p.Lys408Valfs*31, p.Ser404* on MT dynamics and Tau interactions (live-cell fluorescence-microscopy) suggested spared toxic function in p.Lys408Valfs*31, with a regained SH3-binding motif due to the frameshift insertion. Site-directed-mutagenesis, abolishing the p.Lys408Valfs*31 SH3-binding motif, produced MT toxicity. NAP normalized MT activities in the face of all ADNP mutations, although SKIP, being absent the SH3-binding motif, showed reduced efficacy in terms of MT-Tau interactions, as compared with NAP. Lastly, SH3 and multiple ankyrin repeat domains protein 3 (SHANK3), a major autism gene product, interact with the cytoskeleton through an actin-binding motif to modify behavior. Similarly, ELM analysis identified an actin-binding site on ADNP, suggesting direct SH3 and indirect SHANK3/ADNP associations. Actin co-immunoprecipitations from mouse brain extracts showed NAP-mediated normalization of Shank3-Adnp-actin interactions. Furthermore, NAP treatment ameliorated aberrant behavior in mice homozygous for the Shank3 ASD-linked InsG3680 mutation, revealing a fundamental shared mechanism between ADNP and SHANK3, with NAP protective activity reaching out beyond the ADNP syndrome to multiple syndromes involving SHANK3 and actin.

### REFERENCES FOR EXAMPLE 2

1. Pinhasov A, Mandel S, Torchinsky A, Giladi E, Pittel Z, Goldsweig A M, Servoss S J, Brenneman D E, and Gozes I. Activity-dependent neuroprotective protein: a novel gene essential for brain formation. Brain Res Dev Brain Res. 2003,144(1),83.
2. Mandel S, Rechavi G, and Gozes I. Activity-dependent neuroprotective protein (ADNP) differentially interacts with chromatin to regulate genes essential for embryogenesis. Dev Biol. 2007,303(2),814.
3. Malishkevich A, Leyk J, Goldbaum O, Richter-Landsberg C, and Gozes I. ADNP/ADNP2 expression in oligodendrocytes: implication for myelin-related neurodevelopment. J Mol Neurosci. 2015,57(2),304.
4. Merenlender-Wagner A, Malishkevich A, Shemer Z, Udawela M, Gibbons A, Scarr E, Dean B, Levine J, Agam G, and Gozes I. Autophagy has a key role in the pathophysiology of schizophrenia. Mol Psychiatry. 2015,20(1),126.
5. Chu Y, Morfini G A, and Kordower J H. Alterations in Activity-Dependent Neuroprotective Protein in Sporadic and Experimental Parkinson's Disease. J Parkinsons Dis. 2016,6(1),77.
6. Helsmoortel C, Vulto-van Silfhout A T, Coe B P, Vandeweyer G, Rooms L, van den Ende J, Schuurs-Hoeijmakers J H, Marcelis C L, Willemsen M H, Vissers L E, Yntema H G, Bakshi M, Wilson M, Witherspoon K T, Malmgren H, et al. A SWI/SNF-related autism syndrome caused by de novo mutations in ADNP. Nat Genet. 2014,46(4),380.
7. Hacohen-Kleiman G, Sragovich S, Karmon G, Gao A Y L, Grigg I, Pasmanik-Chor M, Le A, Korenkova V, McKinney R A, and Gozes I. Activity-dependent neuroprotective protein deficiency models synaptic and developmental phenotypes of autism-like syndrome. J Clin Invest. 2018,128(11),4956.
8. De Rubeis S, He X, Goldberg A P, Poultney C S, Samocha K, Cicek A E, Kou Y, Liu L, Fromer M, Walker S, Singh T, Klei L, Kosmicki J, Shih-Chen F, Aleksic B, et al. Synaptic, transcriptional and chromatin genes disrupted in autism. Nature. 2014,515(7526),209.
9. Iossifov I, O'Roak B J, Sanders S J, Ronemus M, Krumm N, Levy D, Stessman H A, Witherspoon K T, Vives L, Patterson K E, Smith J D, Paeper B, Nickerson D A, Dea J, Dong S, et al. The contribution of de novo coding mutations to autism spectrum disorder. Nature. 2014,515(7526),216.
10. Yuen R K, Merico D, Cao H, Pellecchia G, Alipanahi B, Thiruvahindrapuram B, Tong X, Sun Y, Cao D, Zhang T, Wu X, Jin X, Zhou Z, Liu X, Nalpathamkalam T, et al. Genome-wide characteristics of de novo mutations in autism. NPJ Genom Med. 2016,1(160271.
11. O'Roak B J, Vives L, Girirajan S, Karakoc E, Krumm N, Coe B P, Levy R, Ko A, Lee C, Smith J D, Turner E H, Stanaway I B, Vernot B, Malig M, Baker C, et al. Sporadic autism exomes reveal a highly interconnected protein network of de novo mutations. Nature. 2012,485(7397),246.
12. Gozes I, Helsmoortel C, Vandeweyer G, Van der Aa N, Kooy F, and Sermone S B. The Compassionate Side of Neuroscience: Tony Sermone's Undiagnosed Genetic Journey—ADNP Mutation. J Mol Neurosci. 2015,56(4),751.
13. Pescosolido M F, Schwede M, Johnson Harrison A, Schmidt M, Gamsiz E D, Chen W S, Donahue J P, Shur N, Jerskey B A, Phornphutkul C, and Morrow E M. Expansion of the clinical phenotype associated with mutations in activity-dependent neuroprotective protein. J Med Genet. 2014,51(9),587.
14. Van Dijck A, Vulto-van Silfhout A T, Cappuyns E, van der Werf I M, Mancini G M, Tzschach A, Bernier R, Gozes I, Eichler E E, Romano C, Lindstrand A, Nordgren A, Consortium A, Kvarnung M, Kleefstra T, et al. Clinical Presentation of a Complex Neurodevelopmental Disorder Caused by Mutations in ADNP. Biol Psychiatry. 2019,85(4),287.
15. Mandel S, and Gozes I. Activity-dependent neuroprotective protein constitutes a novel element in the SWI/SNF chromatin remodeling complex. J Biol Chem. 2007,282(47),34448.
16. Ostapcuk V, Mohn F, Carl S H, Basters A, Hess D, Iesmantavicius V, Lampersberger L, Flemr M, Pandey A, Thoma N H, Betschinger J, and Buhler M. Activity-dependent neuroprotective protein recruits HP1 and CHD4 to control lineage-specifying genes. Nature. 2018,557(7707),739.
17. Gozes I. The cytoskeleton as a drug target for neuroprotection: the case of the autism-mutated ADNP. Biol Chem. 2016,397(3),177.
18. Vulih-Shultzman I, Pinhasov A, Mandel S, Grigoriadis N, Touloumi O, Pittel Z, and Gozes I. Activity-dependent neuroprotective protein snippet NAP reduces tau hyperphosphorylation and enhances learning in a novel transgenic mouse model. J Pharmacol Exp Ther. 2007,323(2),438.
19. Ivashko-Pachima Y, Sayas C L, Malishkevich A, and Gozes I. ADNP/NAP dramatically increase microtubule end-binding protein-Tau interaction: a novel avenue for protection against tauopathy. Mol Psychiatry. 2017,22(9),1335.
20. Sudo H, and Baas P W. Strategies for diminishing katanin-based loss of microtubules in tauopathic neurodegenerative diseases. Hum Mol Genet. 2011,20(4),763.
21. Zemlyak I, Manley N, Sapolsky R, and Gozes I. NAP protects hippocampal neurons against multiple toxins. Peptides. 2007,28(10),2004.
22. Gozes I, Iram T, Maryanovsky E, Arviv C, Rozenberg L, Schirer Y, Giladi E, and Furman-Assaf S. Novel tubulin and tau neuroprotective fragments sharing structural similarities with the drug candidate NAP (Davuentide). J Alzheimers Dis. 2014,40 Suppl 1(S23.
23. Gozes I, Divinski I, and Piltzer I. NAP and D-SAL: neuroprotection against the beta amyloid peptide (1-42). BMC Neurosci. 2008,9 Suppl 3(S3.
24. Oz S, Kapitansky O, Ivashco-Pachima Y, Malishkevich A, Giladi E, Skalka N, Rosin-Arbesfeld R, Mittelman L, Segev O, Hirsch J A, and Gozes I. The NAP motif of activity-dependent neuroprotective protein (ADNP) regulates dendritic spines through microtubule end binding proteins. Mol Psychiatry. 2014,19(10),1115.
25. Oz S, Ivashko-Pachima Y, and Gozes I. The ADNP derived peptide, NAP modulates the tubulin pool: implication for neurotrophic and neuroprotective activities. PLoS One. 2012,7(12),e51458.
26. Smith-Swintosky V L, Gozes I, Brenneman D E, D'Andrea M R, and Plata-Salaman C R. Activity-dependent neurotrophic factor-9 and NAP promote neurite outgrowth in rat hippocampal and cortical cultures. J Mol Neurosci. 2005,25(3),225.
27. Lasser M, Tiber J, and Lowery L A. The Role of the Microtubule Cytoskeleton in Neurodevelopmental Disorders. Front Cell Neurosci. 2018,12(165.
28. Bonini S A, Mastinu A, Ferrari-Toninelli G, and Memo M. Potential Role of Microtubule Stabilizing Agents in Neurodevelopmental Disorders. Int J Mol Sci. 2017,18(8).
29. Chakraborti S, Natarajan K, Curiel J, Janke C, and Liu J. The emerging role of the tubulin code: From the tubulin molecule to neuronal function and disease. Cytoskeleton (Hoboken). 2016,73(10),521.
30. Grigg I, Ivashko-Pachima Y, Hait T A, Korenkovi V, Touloumi O, Lagoudaki R, Van Dijck A, Marusic Z, Anicic M, Vukovic J, Kooy R F, Grigoriadis N, and Gozes I. Tauopathy in the young autistic brain: novel biomarker and therapeutic target. Transl Psychiatry. 2020,10(1),228.
31. Ivashko-Pachima Y, Hadar A, Grigg I, Korenkovi V, Kapitansky O, Karmon G, Gershovits M, Sayas C L, Kooy R F, Attems J, Gurwitz D, and Gozes I. Discovery of autism/intellectual disability somatic mutations in Alzheimer's brains: mutated ADNP cytoskeletal impairments and repair as a case study. Mol Psychiatry. 2019.
32. Schlessinger J. SH2/SH3 signaling proteins. Curr Opin Genet Dev. 1994,4(1),25.
33. Dinkel H, Van Roey K, Michael S, Kumar M, Uyar B, Altenberg B, Milchevskaya V, Schneider M, Kuhn H, Behrendt A, Dahl S L, Damerell V, Diebel S, Kalman S, Klein S, et al. ELM 2016—data update and new functionality of the eukaryotic linear motif resource. Nucleic Acids Res. 2016,44(D1),D294.
34. Reynolds C H, Garwood C J, Wray S, Price C, Kellie S, Perera T, Zvelebil M, Yang A, Sheppard P W, Varndell I M, Hanger D P, and Anderton B H. Phosphorylation regulates tau interactions with Src homology 3 domains of phosphatidylinositol 3-kinase, phospholipase Cgamma1, Grb2, and Src family kinases. J Biol Chem. 2008,283(26),18177.
35. Zhou Y, Kaiser T, Monteiro P, Zhang X, Van der Goes M S, Wang D, Barak B, Zeng M, Li C, Lu C, Wells M, Amaya A, Nguyen S, Lewis M, Sanjana N, et al. Mice with Shank3 Mutations Associated with ASD and Schizophrenia Display Both Shared and Distinct Defects. Neuron. 2016,89(1),147.
36. Sragovich S, Amram N, Yeheskel A, and Gozes I. VIP/PACAP-Based Drug Development: The ADNP/NAP-Derived Mirror Peptides SKIP and D-SKIP Exhibit Distinctive in vivo and in silico Effects. Front Cell Neurosci. 2019,13(589.
37. Kohlenberg T M, Trelles M P, McLarney B, Betancur C, Thurm A, and Kolevzon A. Psychiatric illness and regression in individuals with Phelan-McDermid syndrome. J Neurodev Disord. 2020,12(1),7.
38. Sragovich S, Malishkevich A, Piontkewitz Y, Giladi E, Touloumi O, Lagoudaki R, Grigoriadis N, and Gozes I. The autism/neuroprotection-linked ADNP/NAP regulate the excitatory glutamatergic synapse. Transl Psychiatry. 2019,9(1),2.
39. Sragovich S, Ziv Y, Vaisvaser S, Shomron N, Hendler T, and Gozes I. The autism-mutated ADNP plays a key role in stress response. Transl Psychiatry. 2019,9(1),235.
40. Kapitansky O, Giladi E, Jaljuli I, Bereswill S, Heimesaat M M, and Gozes I. Microbiota changes associated with ADNP deficiencies: rapid indicators for NAP (CP201) treatment of the ADNP syndrome and beyond. J Neural Transm (Vienna). 2020,127(2),251.
41. Willsey H R, Exner C R T, Xu Y, Everitt A, Sun N, Wang B, Dea J, Schmunk G, Zaltsman Y, Teerikorpi N, Kim A, Anderson A S, Shin D, Seyler M, Nowakowski T J, et al. Parallel in vivo analysis of large-effect autism genes implicates cortical neurogenesis and estrogen in risk and resilience. Neuron. 2021.
42. Kazdoba T M, Hagerman R J, Zolkowska D, Rogawski M A, and Crawley J N. Evaluation of the neuroactive steroid ganaxolone on social and repetitive behaviors in the BTBR mouse model of autism. Psychopharmacology (Berl). 2016,233(2),309.
43. Menon S, and Gupton S L. Building Blocks of Functioning Brain: Cytoskeletal Dynamics in Neuronal Development. Int Rev Cell Mol Biol. 2016,322(183.
44. Pacheco A, and Gallo G. Actin filament-microtubule interactions in axon initiation and branching. Brain Res Bull. 2016,126(Pt 3),300.
45. Kirkcaldie M T K, and Dwyer S T. The third wave: Intermediate filaments in the maturing nervous system. Mol Cell Neurosci. 2017,84(68.
46. Witte H, and Bradke F. The role of the cytoskeleton during neuronal polarization. Curr Opin Neurobiol. 2008,18(5),479.
47. Yogev S, Cooper R, Fetter R, Horowitz M, and Shen K. Microtubule Organization Determines Axonal Transport Dynamics. Neuron. 2016,92(2),449.
48. Lowery L A, and Van Vactor D. The trip of the tip: understanding the growth cone machinery. Nat Rev Mol Cell Biol. 2009,10(5),332.
49. Rodriguez O C, Schaefer A W, Mandato C A, Forscher P, Bement W M, and Waterman-Storer C M. Conserved microtubule-actin interactions in cell movement and morphogenesis. Nat Cell Biol. 2003,5(7),599.
50. Deen B, Koldewyn K, Kanwisher N, and Saxe R. Functional Organization of Social Perception and Cognition in the Superior Temporal Sulcus. Cereb Cortex. 2015,25(11),4596.
51. Shih P, Keehn B, Oram J K, Leyden K M, Keown C L, and Muller R A. Functional differentiation of posterior superior temporal sulcus in autism: a functional connectivity magnetic resonance imaging study. Biol Psychiatry. 2011,70(3),270.
52. Barnea-Goraly N, Frazier T W, Piacenza L, Minshew N J, Keshavan M S, Reiss A L, and Hardan A Y. A preliminary longitudinal volumetric MRI study of amygdala and hippocampal volumes in autism. Prog Neuropsychopharmacol Biol Psychiatry. 2014,48(124.
53. Barnea-Goraly N, Kwon H, Menon V, Eliez S, Lotspeich L, and Reiss A L. White matter structure in autism: preliminary evidence from diffusion tensor imaging. Biol Psychiatry. 2004,55(3),323.
54. Weber A M, Egelhoff J C, McKellop J M, and Franz D N. Autism and the cerebellum: evidence from tuberous sclerosis. J Autism Dev Disord. 2000,30(6),511.
55. Chow M L, Pramparo T, Winn M E, Barnes C C, Li H R, Weiss L, Fan J B, Murray S, April C, Belinson H, Fu X D, Wynshaw-Boris A, Schork N J, and Courchesne E. Age-dependent brain gene expression and copy number anomalies in autism suggest distinct pathological processes at young versus mature ages. PLoS Genet. 2012,8(3),e1002592.
56. Tsai P T, Hull C, Chu Y, Greene-Colozzi E, Sadowski A R, Leech J M, Steinberg J, Crawley J N, Regehr W G, and Sahin M. Autistic-like behaviour and cerebellar dysfunction in Purkinje cell Tsc1 mutant mice. Nature. 2012,488(7413),647.
57. Nie D, Di Nardo A, Han J M, Baharanyi H, Kramvis I, Huynh T, Dabora S, Codeluppi S, Pandolfi P P, Pasquale E B, and Sahin M. Tsc2-Rheb signaling regulates EphA-mediated axon guidance. Nat Neurosci. 2010,13(2),163.
58. Lin Y C, Frei J A, Kilander M B, Shen W, and Blatt G J. A Subset of Autism-Associated Genes Regulate the Structural Stability of Neurons. Front Cell Neurosci. 2016,10(263.
59. Jaworski J, Hoogenraad C C, and Akhmanova A. Microtubule plus-end tracking proteins in differentiated mammalian cells. Int J Biochem Cell Biol. 2008,40(4),619.
60. Mukaetova-Ladinska E B, Arnold H, Jaros E, Perry R, and Perry E. Depletion of MAP2 expression and laminar cytoarchitectonic changes in dorsolateral prefrontal cortex in adult autistic individuals. Neuropathol Appl Neurobiol. 2004,30(6),615.
61. Mandel S, Spivak-Pohis I, and Gozes I. ADNP differential nucleus/cytoplasm localization in neurons suggests multiple roles in neuronal differentiation and maintenance. J Mol Neurosci. 2008,35(2),127.
62. Garbern J Y, Neumann M, Trojanowski J Q, Lee V M, Feldman G, Norris J W, Friez M J,
Schwartz C E, Stevenson R, and Sima A A. A mutation affecting the sodium/proton exchanger, SLC9A6, causes mental retardation with tau deposition. Brain. 2010,133(Pt 5),1391.
63. Schirer Y, Malishkevich A, Ophir Y, Lewis J, Giladi E, and Gozes I. Novel marker for the onset of frontotemporal dementia: early increase in activity-dependent neuroprotective protein (ADNP) in the face of Tau mutation. PLoS One. 2014,9(1),e87383.
64. Latapy C, Rioux V, Guitton M J, and Beaulieu J M. Selective deletion of forebrain glycogen synthase kinase 3beta reveals a central role in serotonin-sensitive anxiety and social behaviour. Philos Trans R Soc Lond B Biol Sci. 2012,367(1601),2460.
65. Li L, Fothergill T, Hutchins B I, Dent E W, and Kalil K. Wnt5a evokes cortical axon outgrowth and repulsive guidance by tau mediated reorganization of dynamic microtubules. Dev Neurobiol. 2014.74(8).797.
66. Biswas S, and Kalil K. The Microtubule-Associated Protein Tau Mediates the Organization of Microtubules and Their Dynamic Exploration of Actin-Rich Lamellipodia and Filopodia of Cortical Growth Cones. J Neurosci. 2018,38(2),291.
67. Divinski I, Holtser-Cochav M, Vulih-Schultzman I, Steingart R A, and Gozes I. Peptide neuroprotection through specific interaction with brain tubulin. J Neurochem. 2006,98(3),973.
68. Pascual M, and Guerri C. The peptide NAP promotes neuronal growth and differentiation through extracellular signal-regulated protein kinase and Akt pathways, and protects neurons co-cultured with astrocytes damaged by ethanol. J Neurochem. 2007,103(2),557.
69. Lagreze W A, Pielen A, Steingart R, Schlunck G, Hofmann H D, Gozes I, and Kirsch M. The peptides ADNF-9 and NAP increase survival and neurite outgrowth of rat retinal ganglion cells in vitro. Invest Ophthalmol Vis Sci. 2005,46(3),933.
70. Visochek L, Steingart R A, Vulih-Shultzman I, Klein R, Priel E, Gozes I, and Cohen-Armon M. PolyADP-ribosylation is involved in neurotrophic activity. J Neurosci. 2005,25(32),7420.
71. Bar-Sagi D, Rotin D, Batzer A, Mandiyan V, and Schlessinger J. SH3 domains direct cellular localization of signaling molecules. Cell. 1993,74(1),83.
72. Teyra J, Huang H, Jain S, Guan X, Dong A, Liu Y, Tempel W, Min J, Tong Y, Kim P M, Bader G D, and Sidhu S S. Comprehensive Analysis of the Human SH3 Domain Family Reveals a Wide Variety of Non-canonical Specificities. Structure. 2017,25(10),1598.
73. Kurochkina N, and Guha U. SH3 domains: modules of protein-protein interactions. Biophys Rev. 2013,5(1),29.
74. Su P, Lai T K Y, Lee F H F, Abela A R, Fletcher P J, and Liu F. Disruption of SynGAP-dopamine D1 receptor complexes alters actin and microtubule dynamics and impairs GABAergic interneuron migration. Sci Signal. 2019,12(593).
75. Krapivinsky G, Medina I, Krapivinsky L, Gapon S, and Clapham D E. SynGAP-MUPP1-CaMKII synaptic complexes regulate p38 MAP kinase activity and NMDA receptor-dependent synaptic AMPA receptor potentiation. Neuron. 2004,43(4),563.
76. Wang C C, Held R G, and Hall B J. SynGAP regulates protein synthesis and homeostatic synaptic plasticity in developing cortical networks. PLoS One. 2013,8(12),e83941.
77. Gozes I, Van Dijck A, Hacohen-Kleiman G, Grigg I, Karmon G, Giladi E, Eger M, Gabet Y, Pasmanik-Chor M, Cappuyns E, Elpeleg O, Kooy R F, and Bedrosian-Sermone S. Premature primary tooth eruption in cognitive/motor-delayed ADNP-mutated children. Transl Psychiatry. 2017,7(7),e1166.
78. Sun X, Peng X, Cao Y, Zhou Y, and Sun Y. ADNP promotes neural differentiation by modulating Wnt/beta-catenin signaling. Nat Commun. 2020,11(1),2984.
79. Codocedo J F, Montecinos-Oliva C, and Inestrosa N C. Wnt-related SynGAP1 is a neuroprotective factor of glutamatergic synapses against Abeta oligomers. Front Cell Neurosci. 2015,9(227.
80. Jia L, Pina-Crespo J, and Li Y. Restoring Wnt/beta-catenin signaling is a promising therapeutic strategy for Alzheimer's disease. Mol Brain. 2019,12(1),104.
81. Schindelin J, Arganda-Carreras I, Frise E, Kaynig V, Longair M, Pietzsch T, Preibisch S, Rueden C, Saalfeld S, Schmid B, Tinevez J Y, White D J, Hartenstein V, Eliceiri K, Tomancak P, et al. Fiji: an open-source platform for biological-image analysis. Nat Methods. 2012,9(7),676.
82. Rapsomaniki M A, Kotsantis P, Symeonidou I E, Giakoumakis N N, Taraviras S, and Lygerou Z. easyFRAP: an interactive, easy-to-use tool for qualitative and quantitative analysis of FRAP data. Bioinformatics. 2012,28(13),1800.
83. Koulouras G, Panagopoulos A, Rapsomaniki M A, Giakoumakis N N, Taraviras S, and Lygerou Z. EasyFRAP-web: a web-based tool for the analysis of fluorescence recovery after photobleaching data. Nucleic Acids Res. 2018,46(W1),W467.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety

1-39. (canceled)

40. A method of treating a disease associated with visual evoked potential impairment and/or speech impairment that is not due to vocal disturbance and in which the subject suffers from said visual evoked potential impairment and/or said speech impairment, the method comprising administering to the subject a therapeutically effective amount of an ADNF polypeptide, wherein said ADNF polypeptide has a neurotrophic/neuroprotective activity in an in vitro cortical neuron culture assay, thereby treating the disease in the subject, wherein the disease is not ADNP syndrome.

41. The method of claim 40, wherein the disease is selected from the group consisting of Alzheimer's disease, autism spectrum disorder, neurodegenerative disease, cognitive deficit, mental disorder, cytoskeletal disorder and intellectual disability in which the subject suffers from visual evoked potential impairment and/or speech impairment that is not due to vocal disturbance, neurodegenerative disease, cognitive deficit, mental disorder and cytoskeletal disorder.

42. The method of claim 40, wherein said disease is selected from the group consisting of multiple sclerosis, SYNGAP1 syndrome, POGZ syndrome, CHD8 syndrome, SCN2A syndrome, ARID1B-related syndrome, NRXN1 syndrome, DYRK1A syndrome, GRIN disorder, CHD2 syndrome, Dravet syndrome, Rett syndrome, fragile X syndrome, FOXP1 syndrome, SLC-related disorders, Coffin-Siris syndrome, KMT5B syndrome, PTEN autism syndrome, Okihiro syndrome plus developmental delay, Angelman syndrome, Noonan syndrome, Kleefstra syndrome, and Smith-Magenis syndrome.

43. The method of claim 40, wherein said speech impairment or ability is determined by syntax complexity.

44. The method of claim 40, characterized by at least one of:
(i) said ADNF polypeptide is capable of binding EB1 and/or EB3;
(ii) said ADNF polypeptide comprises an SH3 binding domain;
(iii) said ADNF polypeptide is selected from an ADNF III polypeptide and ADNF I polypeptide.

45. The method of claim 40, characterized by at least one of:
(i) the ADNF III polypeptide comprises an amino acid sequence selected form the group consisting of SEQ ID NOs: 2-22;
(ii) ADNF I polypeptide comprises an amino acid sequence selected form the group consisting of SEQ ID NOs: 24-48;
(iii) said polypeptide is ADNF III having the formula (R¹)ₓ-Asn-Ala-Pro-Val-Ser-Ile-Pro-Gln-(R²)_{y} (SEQ ID NO: 49), in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one; or
(iii) said polypeptide is ADNF I having the formula (R¹)ₓ-Ser-Ala-Leu-Leu-Arg-Ser-Ile-Pro-Ala-(R²)_{y} (SEQ ID NO: 50), or an analogue thereof, in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one;
(iv) the polypeptide comprises at least one D-amino acid;
(v) polypeptide is less than 50 amino acids in length or less than 20 amino acids in length.

46. A method of monitoring efficacy of treatment with an ADNF polypeptide in a subject diagnosed with a disease associated with visual evoked potential impairment and/or speech impairment that is not due to vocal disturbance and in which the subject suffers from said visual evoked potential impairment and/or said speech impairment, wherein the subject has a visual evoked potential impairment and/or a speech impairment that is not due to vocal disturbance, the method comprising determining a visual evoked potential and/or speech ability of the subject following the treatment with the ADNF polypeptide, wherein an improvement in said visual evoked potential and/or said speech ability following the treatment with the ADNF polypeptide indicates the treatment is efficacious.

47. The method of claim 46, wherein the disease is selected from the group consisting of Alzheimer's disease, autism spectrum disorder, neurodegenerative disease, cognitive deficit, mental disorder, cytoskeletal disorder and intellectual disability in which the subject suffers from visual evoked potential impairment and/or speech impairment that is not due to vocal disturbance, optionally wherein said disease is not ADNP syndrome or wherein said disease is an ADNP syndrome.

48. The method of claim 46, wherein said disease is selected from the group consisting of multiple sclerosis, SYNGAP1 syndrome, POGZ syndrome, CHD8 syndrome, SCN2A syndrome, ARID1B-related syndrome, NRXN1 syndrome, DYRK1A syndrome, GRIN disorder, CHD2 syndrome, Dravet syndrome, Rett syndrome, fragile X syndrome, FOXP1 syndrome, SLC-related disorders, Coffin-Siris syndrome, KMT5B syndrome, PTEN autism syndrome, Okihiro syndrome plus developmental delay, Angelman syndrome, Noonan syndrome, Kleefstra syndrome, and Smith-Magenis syndrome.

49. The method of claim 46, wherein said speech impairment or ability is determined by syntax complexity.

50. The method of claim 46, characterized by at least one of:
(i) said ADNF polypeptide is capable of binding EB1 and/or EB3;
(ii) said ADNF polypeptide comprises an SH3 binding domain;
(iii) said ADNF polypeptide is selected from an ADNF III polypeptide and ADNF I polypeptide.

51. The method of claim 46, characterized by at least one of:
(i) the ADNF III polypeptide comprises an amino acid sequence selected form the group consisting of SEQ ID NOs: 2-22;
(ii) ADNF I polypeptide comprises an amino acid sequence selected form the group consisting of SEQ ID NOs: 24-48;
(iii) said polypeptide is ADNF III having the formula (R¹)ₓ-Asn-Ala-Pro-Val-Ser-Ile-Pro-Gln-(R²)_{y} (SEQ ID NO: 49), in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one; or
(iv) said polypeptide is ADNF I having the formula (R¹)ₓ-Ser-Ala-Leu-Leu-Arg-Ser-Ile-Pro-Ala-(R²)_{y} (SEQ ID NO: 50), or an analogue thereof, in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one;
(v) the polypeptide comprises at least one D-amino acid;
(vi) polypeptide is less than 50 amino acids in length or less than 20 amino acids in length.

52. A method of monitoring efficacy of treatment in a subject diagnosed with a disease selected from the group consisting of autism spectrum disorder, intellectual disability, Alzheimer's disease, neurodegenerative disease, cognitive deficit, mental disorder and cytoskeletal disorder, the method comprising determining a level of a marker selected from the group consisting of SMOX, ARRB1, ADCY6, FOXO3, STOM, DNAJB4, TMCC2 and CPXM1 in a biological sample of the subject following the treatment, wherein a decrease in said level of said SMOX, ARRB1, ADCY6, FOXO3, STOM, DNAJB4 and/or TMCC2 and/or an increase in said level of said CPXM1 following the treatment indicates the treatment is efficacious, optionally wherein said disease is an ADNP syndrome.

53. The method of claim 52, wherein said disease is selected from the group consisting of multiple sclerosis, SYNGAP1 syndrome, POGZ syndrome, CHD8 syndrome, SCN2A syndrome, ARID1B-related syndrome, NRXN1 syndrome, DYRK1A syndrome, GRIN disorder, CHD2 syndrome, Dravet syndrome, Rett syndrome, fragile X syndrome, FOXP1 syndrome, SLC-related disorders, Coffin-Siris syndrome, KMT5B syndrome, PTEN autism syndrome, Okihiro syndrome plus developmental delay, Angelman syndrome, Noonan syndrome, Kleefstra syndrome, and Smith-Magenis syndrome.

54. The method of claim 52, characterized by at least one of:
(i) said ADNF polypeptide is capable of binding EB1 and/or EB3;
(ii) said ADNF polypeptide comprises an SH3 binding domain;
(iii) said ADNF polypeptide is selected from an ADNF III polypeptide and ADNF I polypeptide.

55. The method of claim 40, characterized by at least one of:
(i) the ADNF III polypeptide comprises an amino acid sequence selected form the group consisting of SEQ ID NOs: 2-22;
(ii) ADNF I polypeptide comprises an amino acid sequence selected form the group consisting of SEQ ID NOs: 24-48;
(iii) said polypeptide is ADNF III having the formula (R¹)ₓ-Asn-Ala-Pro-Val-Ser-Ile-Pro-Gln-(R²)_{y} (SEQ ID NO: 49), in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one; or
(iv) said polypeptide is ADNF I having the formula (R¹)ₓ-Ser-Ala-Leu-Leu-Arg-Ser-Ile-Pro-Ala-(R²)_{y} (SEQ ID NO: 50), or an analogue thereof, in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one;
(v) the polypeptide comprises at least one D-amino acid;
(vi) polypeptide is less than 50 amino acids in length or less than 20 amino acids in length.

56. The method of claim 55, wherein said biological sample comprises a blood sample.

57. A method of treating a disease associated with SHANK3 in a subject in need thereof, the method comprising administering to the subject a therapeutically effective amount of an ADNF III polypeptide, wherein said ADNF III polypeptide comprises an SH3 binding domain, thereby treating the disease in the subject.

58. The method of claim 57, wherein said disease is Phelan McDermind syndrome.

59. The method of claim 57, wherein the ADNF III polypeptide comprises an amino acid sequence selected form the group consisting of SEQ ID NOs: 2-22.

60. The method of claim 57, wherein said polypeptide is ADNF III having the formula (R¹)ₓ-Asn-Ala-Pro-Val-Ser-Ile-Pro-Gln-(R²)_{y} (SEQ ID NO: 49), in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one; or

61. The method of claim 57, wherein said polypeptide is characterized by at least one of:
(i) the polypeptide comprises at least one D-amino acid;
(ii) polypeptide is less than 50 amino acids in length or less than 20 amino acids in length;
(iii) the polypeptide is attached to a cell penetrating or stabilizing moiety.


SEQUENCE LISTING (53 sequences)

SEQ ID NO: 1 - protein, 1102 aa, Homo sapiens
MFQLPVNNLGSLRKARKTVKKILSDIGLEYCKEHIEDFKQFEPNDFYLKNTTWEDVGLWDPSLTKNQDYRTKPFCCSACPFSSKFFSAYKSHFRNVHSEDFENRILLNCPYCTFNADKKTLETHIKIFHAPNASAPSSSLSTFKDKNKNDGLKPKQADSVEQAVYYCKKCTYRDPLYEIVRKHIYREHFQHVAAPYIAKAGEKSLNGAVPLGSNAREESSIHCKRCLFMPKSYEALVQHVIEDHERIGYQVTAMIGHTNVVVPRSKPLMLIAPKPQDKKSMGLPPRIGSLASGNVRSLPSQQMVNRLSIPKPNLNSTGVNMMSSVHLQQNNYGVKSVGQGYSVGQSMRLGLGGNAPVSIPQQSQSVKQLLPSGNGRSYGLGSEQRSQAPARYSLQSANASSLSSGQLKSPSLSQSQASRVLGQSSSKPAAAATGPPPGNTSSTQKWKICTICNELFPENVYSVHFEKEHKAEKVPAVANYIMKIHNFTSKCLYCNRYLPTDTLLNHMLIHGLSCPYCRSTFNDVEKMAAHMRMVHIDEEMGPKTDSTLSFDLTLQQGSHTNIHLLVTTYNLRDAPAESVAYHAQNNPPVPPKPQPKVQEKADIPVKSSPQAAVPYKKDVGKTLCPLCFSILKGPISDALAHHLRERHQVIQTVHPVEKKLTYKCIHCLGVYTSNMTASTITLHLVHCRGVGKTQNGQDKTNAPSRLNQSPSLAPVKRTYEQMEFPLLKKRKLDDDSDSPSFFEEKPEEPVVLALDPKGHEDDSYEARKSFLTKYFNKQPYPTRREIEKLAASLWLWKSDIASHFSNKRKKCVRDCEKYKPGVLLGFNMKELNKVKHEMDFDAEWLFENHDEKDSRVNASKTADKKLNLGKEDDSSSDSFENLEEESNESGSPFDPVFEVEPKISNDNPEEHVLKVIPEDASESEEKLDQKEDGSKYETIHLTEEPTKLMHNASDSEVDQDDVVEWKDGASPSESGPGSQQVSDFEDNTCEMKPGTWSDESSQSEDARSSKPAAKKKATMQGDREQLKWKNSSYGKVEGFWSKDQSQWKNASENDERLSNPQIEWQNSTIDSEDGEQFDNMTDGVAEPMHGSLAGVKLSSQQA

SEQ ID NO: 2 - protein, 8 aa, Artificial sequence
  Synthetic polypeptide amino acid sequence
NAPVSIPQ

SEQ ID NO: 3 - protein, 18 aa, Artificial sequence
  Synthetic polypeptide amino acid sequence
SVRLGLGGNAPVSIPQQS

SEQ ID NO: 4 - protein, 15 aa, Artificial sequence
  Synthetic polypeptide amino acid sequence
LGLGGNAPVSIPQQS

SEQ ID NO: 5 - protein, 13 aa, Artificial sequence
  Synthetic polypeptide amino acid sequence
LGGNAPVSIPQQS

SEQ ID NO: 6 - protein, 10 aa, Artificial sequence
  Synthetic polypeptide amino acid sequence
GGNAPVSIPQ

SEQ ID NO: 7 - protein, 9 aa, Artificial sequence
  Synthetic polypeptide amino acid sequence
  MOD_RES (1)..(1): ACETYLATION
NAPVSKIPQ

SEQ ID NO: 8 - protein, 9 aa, Artificial sequence
  Synthetic polypeptide amino acid sequence
NAPVSKIPQ

SEQ ID NO: 9 - protein, 9 aa, Artificial sequence
  Synthetic polypeptide amino acid sequence
NAPBSAIPQ

SEQ ID NO: 10 - protein, 9 aa, Artificial sequence
  Synthetic polypeptide amino acid sequence
NAPVSRIPQ

SEQ ID NO: 11 - protein, 9 aa, Artificial sequence
  Synthetic polypeptide amino acid sequence
NAPVTRIPQ

SEQ ID NO: 12 - protein, 9 aa, Artificial sequence
  Synthetic polypeptide amino acid sequence
NAPVAAAAQ

SEQ ID NO: 13 - protein, 8 aa, Artificial sequence
  Synthetic polypeptide amino acid sequence
  MISC_FEATURE (1)..(8): d-amino acid
NAPVSIPQ

SEQ ID NO: 14 - protein, 8 aa, Artificial sequence
  Synthetic polypeptide amino acid sequence
AAPVSIPQ

SEQ ID NO: 15 - protein, 8 aa, Artificial sequence
  Synthetic polypeptide amino acid sequence
NAAVSIPQ

SEQ ID NO: 16 - protein, 8 aa, Artificial sequence
  Synthetic polypeptide amino acid sequence
NAPASIPQ

SEQ ID NO: 17 - protein, 8 aa, Artificial sequence
  Synthetic polypeptide amino acid sequence
NAPVAIPQ

SEQ ID NO: 18 - protein, 8 aa, Artificial sequence
  Synthetic polypeptide amino acid sequence
NAPVSAPQ

SEQ ID NO: 19 - protein, 8 aa, Artificial sequence
  Synthetic polypeptide amino acid sequence
NAPVSIAQ

SEQ ID NO: 20 - protein, 8 aa, Artificial sequence
  Synthetic polypeptide amino acid sequence
NAPVSIPA

SEQ ID NO: 21 - protein, 4 aa, Artificial sequence
  Synthetic polypeptide amino acid sequence
SKIP

SEQ ID NO: 22 - protein, 4 aa, Artificial sequence
  Synthetic polypeptide amino acid sequence
  MOD_RES (1)..(1): ACETYLATION
  MOD_RES (4)..(4): AMIDATION
SKIP

SEQ ID NO: 23 - skipped

SEQ ID NO: 24 - protein, 9 aa, Artificial sequence
  Synthetic polypeptide amino acid sequence
SALLRSIPA

SEQ ID NO: 25 - protein, 14 aa, Artificial sequence
  Synthetic polypeptide amino acid sequence
VAGGGSALLRSIPA

SEQ ID NO: 26 - protein, 19 aa, Artificial sequence
  Synthetic polypeptide amino acid sequence
VEEGIVLGGGSALLRSIPA

SEQ ID NO: 27 - protein, 14 aa, Artificial sequence
  Synthetic polypeptide amino acid sequence
VLGGGCALLRCIPA

SEQ ID NO: 28 - protein, 8 aa, Artificial sequence
  Synthetic polypeptide amino acid sequence
ALLRSIPA

SEQ ID NO: 29 - protein, 10 aa, Artificial sequence
  Synthetic polypeptide amino acid sequence
GSALLRSIPA

SEQ ID NO: 30 - protein, 6 aa, Artificial sequence
  Synthetic polypeptide amino acid sequence
SALLRS

SEQ ID NO: 31 - protein, 10 aa, Artificial sequence
  Synthetic polypeptide amino acid sequence
GSALLRSIPA

SEQ ID NO: 32 - protein, 12 aa, Artificial sequence
  Synthetic polypeptide amino acid sequence
VAGGGSALLRSI

SEQ ID NO: 33 - protein, 10 aa, Artificial sequence
  Synthetic polypeptide amino acid sequence
VLGGGSALLR

SEQ ID NO: 34 - protein, 9 aa, Artificial sequence
  Synthetic polypeptide amino acid sequence
VLGGGSALL

SEQ ID NO: 35 - protein, 8 aa, Artificial sequence
  Synthetic polypeptide amino acid sequence
VAGGGSAL

SEQ ID NO: 36 - protein, 9 aa, Artificial sequence
  Synthetic polypeptide amino acid sequence
  MISC_FEATURE (1)..(9): d-amino acid
SALLRSIPA

SEQ ID NO: 37 - protein, 9 aa, Artificial sequence
  Synthetic polypeptide amino acid sequence
AALLRSIPA

SEQ ID NO: 38 - protein, 9 aa, Artificial sequence
  Synthetic polypeptide amino acid sequence
SAALRSIPA

SEQ ID NO: 39 - protein, 9 aa, Artificial sequence
  Synthetic polypeptide amino acid sequence
SALARSIPA

SEQ ID NO: 40 - protein, 9 aa, Artificial sequence
  Synthetic polypeptide amino acid sequence
SALLASIPA

SEQ ID NO: 41 - protein, 9 aa, Artificial sequence
  Synthetic polypeptide amino acid sequence
SALLRAIPA

SEQ ID NO: 42 - protein, 9 aa, Artificial sequence
  Synthetic polypeptide amino acid sequence
SALLRSAPA

SEQ ID NO: 43 - protein, 9 aa, Artificial sequence
  Synthetic polypeptide amino acid sequence
SALLRSIAA

SEQ ID NO: 44 - protein, 26 aa, Artificial sequence
  Synthetic polypeptide amino acid sequence
SALLRSIPAPAGASRLLLLTGEIDLP

SEQ ID NO: 45 - protein, 14 aa, Artificial sequence
  Synthetic polypeptide amino acid sequence
VLGGGSALLRSIPA

SEQ ID NO: 46 - protein, 13 aa, Artificial sequence
  Synthetic polypeptide amino acid sequence
LGGGSALLRSIPA

SEQ ID NO: 47 - protein, 12 aa, Artificial sequence
  Synthetic polypeptide amino acid sequence
GGGSALLRSIPA

SEQ ID NO: 48 - protein, 11 aa, Artificial sequence
  Synthetic polypeptide amino acid sequence
GGSALLRSIPA

SEQ ID NO: 49 - protein, 88 aa, Artificial sequence
  Synthetic polypeptide amino acid sequence
  misc_feature (1)..(40): Xaa can be any naturally occurring amino acid
  misc_feature (49)..(88): Xaa can be any naturally occurring amino acid
XXXXXXXXXXXXXXXXXXXXXXXXXXXXXXXXXXXXXXXXNAPVSIPQXXXXXXXXXXXXXXXXXXXXXXXXXXXXXXXXXXXXXXXX

SEQ ID NO: 50 - protein, 89 aa, Artificial sequence
  Synthetic polypeptide amino acid sequence
  misc_feature (1)..(40): Xaa can be any naturally occurring amino acid
  misc_feature (50)..(89): Xaa can be any naturally occurring amino acid
XXXXXXXXXXXXXXXXXXXXXXXXXXXXXXXXXXXXXXXXSALLRSIPAXXXXXXXXXXXXXXXXXXXXXXXXXXXXXXXXXXXXXXXX

SEQ ID NO: 51 - DNA, 107 bp, Artificial Sequence
  DNA sequence in a schematic representation
tcgggccagttaaagtctccttccctctctcagtcacaggcatccagagtgttaggtcagtccagttccaaacctgctgcagctgccacaggccctcccccaggtaa

SEQ ID NO: 52 - protein, 30 aa, Artificial Sequence
  AA sequence in a schematic representation
VSFPLSVTGIQSVRSVQFQTCCSCHRPSPR

SEQ ID NO: 53 - protein, 36 aa, Artificial Sequence
  AA sequence in a schematic representation
SGQLKSPSLSQSQASRVLGQSSSKPAAAATGPPPGN